(19) United States
(12) Patent Application Publication
(10) Pub. No.: US 2024/0269128 A1
(43) Pub. Date: Aug. 15, 2024

(54) COMPOUNDS FOR TREATING OR PREVENTING ALZHEIMER'S DISEASE

(71) Applicant: THE UNITED STATES OF AMERICA, as represented by the Secretary, Department of Health and Human, Bethesda, MD (US)

(72) Inventors: Madhav Thambisetty, Ellicott City, MD (US); Myriam Gorospe, Ellicott City, MD (US); Carlos Anerillas, Baltimore, MD (US)

(73) Assignee: THE UNITED STATES OF AMERICA, as represented by the Secretary, Department of Health and Human, Bethesda, MD (US)

(21) Appl. No.: 18/627,191

(22) Filed: Apr. 4, 2024

Related U.S. Application Data
(63) Continuation-in-part of application No. PCT/US2022/046018, filed on Oct. 7, 2022.
(60) Provisional application No. 63/253,992, filed on Oct. 8, 2021.

(51) Int. Cl. A61K 31/4706; A61K 31/18; A61K 31/404; A61K 31/4745; A61K 31/505; A61K 31/506; A61K 31/575; A61K 31/7024; A61P 25/28

(52) U.S. Cl. CPC A61K 31/4706; A61K 31/18; A61K 31/404; A61K 31/4745; A61K 31/505; A61K 31/506; A61K 31/575; A61K 31/7024; A61P 25/28

(57) ABSTRACT

A subject is administered an amount of an active agent effective to at least partially normalize an aberrant level of one or more indicators, wherein the indicators comprise extracellular amyloid beta concentration, tau phosphorylation, neuroinflammation, or any combination thereof. The subject may be diagnosed with Alzheimer's disease or may be identified as being at risk of developing Alzheimer's disease.

### CROSS-REFERENCE TO RELATED APPLICATIONS

This application is a continuation-in-part of PCT Application No. PCT/US2022/046018, filed Oct. 7, 2022, which was published in English under PCT Article 21(2), which in turn claims the benefit of the earlier filing date of U.S. Provisional Application No. 63/253,992, filed Oct. 8, 2021, which is incorporated by reference in its entirety herein.

### ACKNOWLEDGMENT OF GOVERNMENT SUPPORT

This invention was made with government support under Project No. Z01 AG000436-03 awarded by the National Institutes of Health. The government has certain rights in the invention.

### FIELD

This disclosure concerns identification of molecular targets associated with Alzheimer's disease and compounds for modifying pathological processes associated with Alzheimer's disease.

### BACKGROUND

The ε4 allele of the apolipoprotein-E (APOE) gene is the most robust genetic risk factor for sporadic or late-onset AD. Heterozygous carriers of the ε4 allele are at 3-4 times greater risk of AD, while homozygous individuals are at 10 times greater risk relative to non-carriers (Farrer et al., JAMA 1997, 278:1349-1356; Neu et al., JAMA Neurol 2017, 74:1178-1189; Genin et al., Mol Psychiatry 2011, 16:903-907). APOE ε4 carriers also have an earlier age-at-onset AD (Bando et al., BMC Neurol 2008, 8:9). Although discovered more than two decades ago (Corder et al., Science 1993, 261:921-923), the precise mechanisms mediating APOE ε4-associated risk of AD remain unclear, and the promise of APOE-based AD treatments remains unrealized.

APOE ε4 carriers accumulate AD neuropathology early in adulthood. A neuroimaging meta-analysis showed that 15% of non-demented APOE ε4 homozygous individuals showed evidence of cerebral amyloid accumulation at 40 years of age (Jansen et al., JAMA 2015, 313:1924-1938). In a recent post-mortem autopsy study of young APOE ε4 carriers, Pletnikova et al. found that more than 40% of ε4 heterozygous and 80% of ε4 homozygous individuals between 40 and 49 years of age had diffuse brain amyloid plaques, compared to less than 1% of non-carriers (Neurobiol Aging 2018, 71:72-80). Additionally, a study of regional cerebral glucose metabolism demonstrated that young, cognitively normal APOE ε4 carriers between 20-39 years showed regional patterns of hypometabolism similar to those observed in patients with AD (Reiman et al., PNAS USA 2004, 101:284-289). Similarly, structural imaging studies have shown that among individuals under the age of 40, APOE ε4 carriers have reduced cortical thickness (Shaw et al., Lancet Neurol 2007, 6:494-500), reduced gray matter volume, and worse cognitive performance (Nao et al., Front Hum Neurosci 2017, 11:346).

This suggests that APOE ε4 contributes to AD risk over the course of several decades prior to disease onset. The underlying biological pathways that connect APOE genotype with the development of pathology that eventually leads to AD, however, have remained unknown. Identifying the precise biological mechanisms that operate in young APOE ε4 carriers to accelerate AD pathogenesis will aid in understanding APOE-related AD risk as well as to the eventual development of AD prevention and/or treatments.

### SUMMARY

This disclosure concerns embodiments of compounds and molecular targets for treating and/or preventing Alzheimer's disease (AD). In some embodiments, the compounds at least partially normalize an abnormal pathological characteristic of AD and/or AD risk.

In some embodiments, a method includes administering to a subject an amount of an active agent effective to at least partially normalize an aberrant level of one or more indicators in the brain, cerebrospinal fluid, and/or blood, wherein the indicators comprise extracellular amyloid beta (Aβ) concentration, tau phosphorylation, neuroinflammation, plasma neurofilament light chain protein (NfL) concentration, hippocampal synaptic plasticity, or any combination thereof. In some embodiments, normalizing the aberrant level of the one or more indicators reduces extracellular Aβ concentration, decreases tau phosphorylation, reduces neuroinflammation, reduces plasma NfL concentration, or any combination thereof. In certain implementations, the active agent at least partially normalizes aberrant levels of two or three of the indicators.

In any of the foregoing or following embodiments, the active agent may comprise C188-9 (TTI-101, N-[4-hydroxy-3-(2-hydroxynaphthalen-1-yl)naphthalen-1-yl]-4-methoxybenzenesulfonamide), dasatinib, hydroxychloroquine, MLS-0437605 (N-4-fluoro-1,3-benzothiazol-2-71)-5-(4-methoxyphenyl)-1,3,4-oxadiazol-2-amine), a methyl-β-d-galactomalonyl phenyl ester, irinotecan, pyrimethamine, TTI-102, tauroursodeoxycholic acid (TUDCA), 3,3′-diindolylmethane (arundine, DIM), or any combination thereof. In some examples, the active agent comprises C188-9, dasatinib, hydroxychloroquine, TUDCA, DIM or any combination thereof.

In any of the foregoing or following embodiments, the method may further include receiving data comprising an initial level of at least one of the indicators prior to administering the active agent to the subject. In any of the foregoing or following embodiments, the method may further include receiving data comprising a post-administration level of at least one of the indicators following administration of the active agent to the subject, and selecting an adjusted amount of the active agent for administration to the subject based at least in part on the post-administration level.

In some embodiments, the subject is diagnosed as having Alzheimer's disease (AD) prior to administering the active agent. In some implementations, the method further includes identifying the subject as being at risk of developing AD by (i) identifying the subject as being an APOE ε4 carrier, or (ii) identifying the subject as having an elevated level of the one or more indicators relative to a normal level of the one or more indicators in the brain, or (iii) identifying the subject as having an aberrant level of the one or more indicators in cerebrospinal fluid and/or blood assays, or (iv) any combination of (i), (ii), and (iii). In certain embodiments, the active agent is administered to the subject prophylactically in the absence of any cognitive, behavioral, mood, or psychological signs or symptoms of AD.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are schematic diagrams of a three-stage study to characterize underlying biological pathways that connect the APOE genotype with the development of pathology leading to Alzheimer's disease (AD); BLSA—Baltimore Longitudinal Study of Aging; YAPS—Young APOE Postmortem Study; CN—cognitively normal; ROS—Religious Orders Study; RNA—ribonucleic acid; WB—western blot; IHC—immunohistochemistry.

FIGS. 2A-2D show results identifying an incipient AD proteomic signature. FIG. 2A shows results of separate proportional odds models. FIG. 2B shows volcano plots highlighting proteins in BLSA and ROS used to define the 120 protein AD proteomic signature. FIG. 2C shows volcano plots highlighting proteins from the 120 protein AD proteomic signature that overlap with proteins differentially abundant in YAPS ITG or MFG (i.e., the 25-protein incipient AD signature). FIG. 2D shows differences in protein levels in each cohort for the 25 proteins included in the incipient AD proteomic signature; for the proteins significant in both the ITG and MFG, the more significant brain region (i.e., smaller p-value in the proportional odds model) was visualized. AD: Alzheimer's disease; BLSA: Baltimore Longitudinal Study of Aging; ROS: Religious Orders Study; YAPS: Young APOE Postmortem Study; CN: cognitively normal; ITG: inferior temporal gyrus; MFG: middle frontal gyrus; OR: odds ratio. * indicates opposite direction of abundance in AD/CN between BLSA and ROS.

FIGS. 3A-3C show associations between the incipient AD proteomic signature with severity of AD pathology and longitudinal trajectories of ante-mortem cognitive performance. FIGS. 3A and 3B, respectively show Braak and CERAD scores in the BLSA and ROS cohorts in either the ITG or MFG. FIG. 3C shows associations between proteins comprising the incipient AD proteomic signature and longitudinal trajectories in MMSE scores in AD individuals in the BLSA and ROS in the ITG or MFG. BLSA: Baltimore Longitudinal Study of Aging; ROS: Religious Orders Study; CERAD: Consortium to Establish a Registry for Alzheimer's Disease; AD: Alzheimer's disease; ITG: inferior temporal gyrus; MFG: middle frontal gyrus; MMSE: Mini Mental State Examination.

FIG. 4 shows results of GSEA analyses for the 17 gene sets identified as dysregulated in both YAPS and at least one AD cohort. YAPS: Young APOE Postmortem Study; GSEA: gene set enrichment analysis.

FIGS. 5A-5B show results of primary validation analysis performed using western blotting in 3xTg-AD transgenic mice (AD) compared to wild type (WT) (FIG. 5A) and human brain tissue samples comparing AD to CN (FIG. 5B).

FIG. 6 is western blot images 3xTg-AD mouse brain samples showing results of primary validation analyses in 3xTg-AD transgenic mice (AD, n=6) compared to wild type (WT, n=6).

FIG. 7 is western blot images in BLSA MFG brain samples showing results of primary validation analyses using samples from AD (n=11) and CN (n-11) individuals.

FIGS. 8A-8B are representative images of DUSP3 antibody co-stained with various subcellular markers in neurons within the inferior parietal cortex showing DUSP3 immunocolocalization with subcellular markers. FIG. 8A shows low magnification images of DUSP3 and MAP2 (neuronal marker). FIG. 8B shows DUSP3 and SYP (synaptophysin-presynaptic marker) (top row), DUSP3 and PSD95 (excitatory postsynaptic marker) (middle row), and DUSP3 and Gephyrin (inhibitory postsynaptic marker) (bottom row). White arrows indicate the examples of colocalized immunoreactivity between antibodies. Scale bars, 50 μm (8A), 10 μm (8B). Images in FIG. 8B were captured at 100× magnification, with inset images cropped from the larger image.

FIGS. 9A-9B are representative images of LGALS8 antibody co-stained with various subcellular markers in inferior parietal cortex showing LGALS8 immunocolocalization with subcellular markers. FIG. 9A shows low magnification images of LGALS8 and MAP2 (neuronal marker). FIG. 9B shows LGALS8 and MTCO1 (mitochondrial marker) (upper row), LGALS8 and Rab5 (early endosomal marker) (middle row), and LGALS8 and LAMP1 (lysosomal marker) (bottom row). Scale bars, 50 μm (8A), 20 μm (8B).

FIGS. 10A-10B show the validation process (FIG. 10A) and results of validation analyses carried out with publicly available proteomic and transcriptomic datasets (FIG. 10B). Colored squares represent proteins/genes that were differentially abundant/expressed between AD and CN or 5xFAD versus WT mice. Gray squares indicate proteins/transcripts that were not quantified (N.Q.). White squares indicate proteins/transcripts that were not significantly (N.S.) differentially abundant/expressed between AD and CN or 5xFAD versus WT mice. Significance was defined as p<0.05. AD: Alzheimer's disease; BLSA: Baltimore Longitudinal Study of Aging; CN: cognitively normal; ROS: Religious Orders Study; WT: wild type; YAPS: Young APOE Postmortem Study; ROSMAP: Religious Orders Study and Rush Memory and Aging Project; scRNA: single-cell ribonucleic acid; LC-MS/MS: liquid chromatography with tandem mass spectrometry.

FIGS. 11A-11B show phenotypic screening results of candidate AD drug targets. FIG. 11A shows that the STAT3 inhibitor C188-9 rescued 3 AD phenotypes: LPS-induced neuroinflammation (by lowering IL-6 and IL-1β levels), tau phosphorylation (by lowering ptau levels), and lowering secretion of Aβ42 and reducing Aβ42: Aβ40 ratio). FIG. 11B shows that the YES1/FYN inhibitor dasatinib rescued one AD phenotype: by lowering tau phosphorylation (ptau levels). Blue bars indicate the comparison group: either LPS or the VC. Orange bars indicate 3 increasing concentrations for treatment with C188-9 or dasatinib. Values were compared by one-way ANOVA followed by Dunnetts multiple comparison test and significant differences were indicated (*p<0.05; ** p<0.01; *** p<0.001). LPS: lipopolysaccharide; VC: vehicular control (0.1% DMSO); ptau: phosphorylated tau 231; AU: arbitrary units.

FIG. 12 is Western blot images evaluating whether hydroxychloroquine (HCQ) treatment (50 μM; 48 hours of treatment) alters level of p-STAT3 at two phosphorylation sites: Tyr705 and Ser727. After HCQ treatment, levels of p-STAT3 (Tyr705 and Ser727) were reduced without alteration in total STAT3 levels in HMC3 cells, 132N1 cells and mouse cortical neurons. p-STAT3: phosphorylated STAT3; HCQ: hydroxychloroquine; Tyr705: tyrosine 705; Ser727: serine 727.

FIGS. 13A-13C show that HCQ rescues molecular phenotypes relevant to AD. FIG. 13A shows greater Aβ₁₋₄₂ clearance in microglia. FIG. 13B shows reduction in tau phosphorylation in neuroblastoma cells overexpressing human mutant tau. FIG. 13C shows lowering of bacterial lipopolysaccharide induced neuroinflammation in microglia. Data are presented as bar graphs with group mean and error bars representing standard deviation (SD). Individual values are shown as dots (n=6 per group). Values were compared by one-way ANOVA followed by Dunnetts multiple comparison test versus vehicle control (VC): * p<0.05; ** p<0.01; *** p<0.001. HCQ: hydroxychloroquine; AU: arbitrary units; VC: vehicle control (0.1% DMSO); LPS: lipopolysaccharide, RI: reference item; dexamethasone 10 μM.

FIGS. 14A-14G show that rescue of AD phenotypes by HCQ is associated with STAT3 inactivation. FIGS. 14A and 14B show increase in Aβ₁₋₄₂ clearance in microglia. FIGS. 14C and 14D show reduction in tau phosphorylation. FIGS. 14E-14G show lowering of bacterial lipopolysaccharide-induced neuroinflammation. Group differences were evaluated using two-sample t-tests: * p<0.05; ** p<0.01; VC: vehicle control; HCQ: hydroxychloroquine; VC: vehicle control (0.1% DMSO); p-STAT3: phosphorylated STAT3; Tyr705: tyrosine 705; Ser727: serine 727.

FIGS. 15A-15H show that HCQ rescues late-long-term potentiation (LTP) in hippocampal CA1 synapses of APP/PS1 mice. FIG. 15A is a schematic representation of a hippocampal slice with electrodes located in the CA1 region. ‘Rec’ represents the recording electrode positioned in the CA1 region flanked by two stimulating electrodes represented as S1 and S2 in the stratum radiatum to stimulate two independent pathways to a single neuronal population in Schaffer collateral pathway (sc). FIG. 15B shows induction of late-LTP by STET in synaptic input S1 in WT mice resulted in a potentiation that remained stable for 180 min (filled circles, n=7). FIG. 15C shows induction of late-LTP by STET in synaptic input S1 in APP/PS1 mice resulted only in early-LTP in S1 (filled circles, n=6). FIG. 15D shows treatment of hippocampal slices with 25 μM HCQ resulted in late-LTP in S1 in APP/PS1 mice (filled circles, n=8) that was however significantly lower than WT late-LTP (D vs B). FIG. 15E shows that treatment of hippocampal slices with 50 μM HCQ resulted in late-LTP in S1 in APP/PS1 mice (filled circles, n=7) that was similar to WT late-LTP (E vs B). FIG. 15F shows that treatment of hippocampal slices from wild type mice with 50 μM HCQ does not alter L-LTP compared to untreated wild type hippocampal slices, indicating that HCQ affects synaptic plasticity only in the APP/PS1 transgenic mice. In FIGS. 15B-15F control input S2 remained stable throughout the recording (open blue circles). In FIG. 15G, a comparison of input-output curves showed no significant change between WT and APP/PS1 before and after HCQ application. In FIG. 15H, a comparison of Paired Pulse Ratio (PPR) also revealed no significant change in Paired Pulse Facilitation (PPF) ratio between WT and APP/PS1 mice before and after HCQ application (n=12). Error bars in all the graphs indicate ±SEM. Analog traces represent typical fEPSPs of inputs S1 and S2, recorded 15 min before (dotted line), 30 min after (dashed line), and 180 min (solid line) after tetanization in S1 and the corresponding time points in S2. Three solid arrows represent the time of induction of late-LTP by STET. Solid rectangular bar in FIGS. 15D-15F represents the time of application of HCQ. Scale bars: vertical, 2 mV; horizontal, 3 ms.

FIGS. 16A-16B show that HCQ reduces levels of hippocampal p-STAT3 in APP/PS1 mice. FIG. 16A is a western blot analysis of hippocampal p-STAT3 (Tyr705) and total STAT3 levels between WT, APP/PS1, WT+50 μM HCQ and APP/PS1+50 μM HCQ mice (N=3 for each group). Levels of p-STAT3 (Tyr705) were significantly higher in APP/PS1 mice compared to WT mice and were similar between WT and WT+50 μM HCQ mice. FIG. 16B shows p-STAT3/total STAT3 ratio (N=3 for each group). The values of the individual groups were calculated in relation to the control group, while tubulin serves as a loading control. Asterisk indicates significant differences between groups (two-way ANOVA, * P<0.05, ** P<0.01, *** P<0.001 and **** P<0.0001). Error bars indicate ±SEM.

FIG. 17 shows analyses of cumulative incidence of Alzheimer's and related dementia (ADRD) in rheumatoid arthritis patients treated with methotrexate or HCQ; Medicare data 2007-2017. The four analyses were designed to address various uncertainties associated with claims-based analyses of ADRD risk including: Analysis 1: ‘As-treated’ follow-up approach; Analysis 2: ‘As-started’ follow-up approach incorporating a 6-month induction period; Analysis 3: Incorporating a 6-month ‘symptom to diagnosis’ period' and Analysis 4: Alternate outcome definition. HCQ: hydroxychloroquine; MTX: methotrexate.

FIG. 18 shows the comparative risk of ADRD in rheumatoid arthritis patients treated with HCQ vs. MTX; Medicare data 2007-2017. The four analyses are those described in FIG. 17. HCQ: hydroxychloroquine; MTX: methotrexate; PS: propensity score.

FIGS. 19A and 19B are fluorescence images (FIG. 19A) and a bar graph (FIG. 19B) showing that HCQ pretreatment enhances microglial uptake of Aβ₁₋₄₂ preferentially into acidic cellular compartments such as lysosomes.

FIG. 20 shows that HCQ lowers release of several cytokines in microglial cells from a transgenic AD mouse model.

FIG. 21 shows levels of Aβ1-38, Aβ1-40, and Aβ1-42 in supernatant after 24 hours treatment with tauroursodeoxycholic Acid (TUDCA) at TUDCA concentrations of 100 μM (C1), 10 μM (C2), and 1.0 μM (C3).

FIG. 22 shows levels of Aβ1-38, Aβ1-40, and Aβ1-42 in supernatant after 24 hours treatment with HCQ and TUDCA at concentrations of 25 μM HCQ/0.1 μM TUDCA (C1), 2.5 μM HCQ/100 μM TUDCA (C2), and 2.5 μM HCQ/10 μM TUDCA (C3).

FIG. 23 shows aggregated performance of TUDCA and DIM (arundine) in 9 experiments where performance is characterized by the posterior probability of a protective effect. The probabilities were calculated by averaging the probabilities across all assays in any given experiment. Scores greater than zero indicate a protective effect probability, with higher scores indicating stronger evidence for a protective effect.

FIGS. 24A-24E show plasma NfL measured in wild-type (WT) (FIG. 24A) and 5xFAD mice (FIG. 24B) treated with saline over 10 weeks, as well as 5xFAD mice treated with HCQ (FIG. 24C), HCQ nano (FIG. 24D), saline (FIG. 24E), and TUDCA (FIG. 24F).

FIG. 25 shows the estimated effects of the drugs of FIGS. 24C-24F.

FIGS. 26A and 26B show that racemic HCQ free base has limited effectiveness in rescuing synaptic plasticity in APP/PS1 mice at 50 μM (FIG. 26A) and is partially effective at 25 μM (FIG. 26B).

FIGS. 27A and 27B show that application of 10 μM DIM rescues LTP in APP/PS1 slices (FIG. 27A) and a 3-hour pre-incubation with 10 μM DIM no longer results in rescue of late LTP in APP/PS1 slices (FIG. 27B).

FIG. 28 shows that a mixture of 40 μM HCQ sulfate and 10 μM DIM had a deleterious effect on synaptic plasticity in APP/PS1 slices.

FIG. 29 shows that 10 μM TUDCA had a deleterious effect on synaptic plasticity in APP/PS1 slices.

FIGS. 30A-30D show quantification of Aβ40 and Aβ42 in DEA soluble and FA soluble fraction of the cortex: Aβ40 in FA soluble fraction (FIG. 30A); Aβ42 in FA soluble fraction (FIG. 30B); Aβ40 in DEA soluble fraction (FIG. 30C); Aβ42 in DEA soluble fraction (FIG. 30D). Data are given as pg per mg tissue and represented as group means +SEM (n=6 per group), Statistics: one-way ANOVA+Dunnett's multiple comparisons test. All data were analyzed using group A as reference group for pairwise comparisons. * p<0.05; ** p<0.01; *** P<0.001. Groups: A—transgenic mice (t.g.), saline; C—t.g., TUDCA 500 mg/kg+HCQ 100 mg/kg; D—t.g., DIM 50 mg/kg; E—t.g., DIM 100 mg/kg; F—t.g., DIM 200 mg/kg; G—t.g., vehicle.

FIGS. 31A and 31B show effect of HCQ sulfate/DIM on Aβ1-42 phagocytosis. FIG. 31A shows the Aβ1-42 level in the supernatant, and FIG. 31B shows the Aβ1-42 level in the cell lysates. Data are presented as bar graph with group mean+SEM (n=6 per group). VC=vehicle control 0.1% DMSO, c1=25 μM HCQ/10 μM DIM, c2=25 μM HCQ/3 μM DIM, c3=10 μM HCQ/10 μM DIM. Statistical Analysis: One-way ANOVA followed by Bonferroni's multiple comparison test versus VC: *p<0.05; ** p<0.01; *** p<0.001.

FIGS. 32A-32D show effects of HCQ sulfate/DIM on Aβ1-38 (FIG. 32A), Aβ1-40 (FIG. 32B), Aβ1-42 (FIG. 32C), and MTT (FIG. 32D) in supernatant of H4h-APP cells after 24 hours. Viability assessed with MTT assay given as % of the vehicle control. Data are presented as bar graph with group mean+SEM (n=6 per group). VC=vehicle control H₂O; c1=25 μM HCQ/10 μM DIM, c2=25 μM HCQ/3 μM DIM, c3=10 μM HCQ/10 μM DIM. Statistical Analysis: One-way ANOVA followed by Dunnett's multiple comparison test versus vehicle control (VC): * p<0.05; ** p<0.01 ;*** p<0.001.

FIGS. 33A-33C show effects of HCQ sulfate/DIM on total Tau (FIG. 33A) and pTau231 (FIG. 33B) as well as pTau:total Tau ratio (FIG. 33C) in cell lysates after 24 h treatment with HCQ sulfate/DIM or RI (CHIR, GSK3 inhibitor). Total Tau level given as pg/μg total protein, pTau231 level as arbitrary unit (AU), and the ratio of pTau231 to total Tau multiplied with 10000. c1=25 μM HCQ/10 μM DIM, c2=25 μM HCQ/3 μM DIM, c3=10 μM HCQ/10 μM DIM. Data are presented as bar graph with group mean +SEM (n=6 per group). VC=vehicle control 0.1% DMSO. Statistical Analysis: One-way ANOVA followed by Bonferroni's multiple comparison test versus vehicle control (VC): * p<0.05; ** p<0.01; *** p<0.001.

FIGS. 34A-34F show levels of secreted cytokines as well as cell viability after 24 h treatment in LPS-stimulated BV2 microglial cells: TNF-α (FIG. 34A), IL-1β (FIG. 34B) IL-6 (FIG. 34C), KC/GRO (FIG. 34D), IL-10 assessed with MSD V-plex (FIG. 34E) given as pg/mL, cell viability assessed with MTT assay given as percent of LPS control (FIG. 34F). Data are presented as bar graph with group mean+SEM (n=6 per group). LPS=stimulated vehicle control, VC=unstimulated vehicle control H₂O; c1=25 μM HCQ/10 μM DIM, c2=25 μM HCQ/3 μM DIM, c3=10 μM HCQ/10 μM DIM. Statistical Analysis: One-way ANOVA followed by Dunnett's multiple comparison test versus stimulated vehicle control (LPS): * p<0.05; ** p<0.01 ;*** p<0.001.

### DETAILED DESCRIPTION

This disclosure concerns embodiments of compounds and molecular targets for treating and/or preventing Alzheimer's disease (AD). In some embodiments, the compounds at least partially normalize an abnormal pathological characteristic of AD and/or AD risk. The discoveries of an AD proteomic signature and an incipient AD proteomic signature also are disclosed. The proteomic signatures are useful for identifying a deceased subject as having had AD.

### I. DEFINITIONS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, percentages, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Aberrant: As used herein, the term “aberrant” refers to deviating from an accepted standard value. As used herein with respect to a biomarker, the term “aberrant level” refers to a level that deviates from a range considered to represent normal levels of the biomarker.

Active agent: A drug, medicament, pharmaceutical, therapeutic agent, nutraceutical, or other compound that may be administered to a subject to effect a change, such as treatment, amelioration, or prevention of a disease or disorder or at least one symptom associated therewith. The active agent may be a “small molecule,” generally having a molecular weight of about 2000 daltons or less. The active agent may also be a “biological active agent.” Biological active agents include proteins, antibodies, antibody fragments, peptides, oligonucleotides, vaccines, and various derivatives of such materials.

Administration: To provide or give a subject an agent, such as one or more compounds provided herein, by any effective route. Exemplary routes of administration include, but are not limited to, oral, injection (such as subcutaneous, intramuscular, intradermal, intraperitoneal, intravenous, intraosseous, intracerebroventricular, intrathecal, and intratumoral), sublingual, rectal, transdermal, intranasal, vaginal and inhalation routes.

Effective amount: An amount sufficient to provide a beneficial, or therapeutic, effect to a subject or a given percentage of subjects, such as an amount effective to elicit a desired biological or medical response in a tissue, system, subject or patient; to treat a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the age of the patient to be treated, and the like.

Indicator: As used herein, the term “indicator” refers to a measurable component indicative of a pathology, such as a component indicative of Alzheimer's disease pathology.

Normalize: As used herein, the term “normalize” means to move a value (e.g., a level of a biomarker) closer to a “normal” or accepted standard value of the biomarker.

Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compositions and additional pharmaceutical agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a pill, vial, bottle, or syringe.

Subject: An animal (human or non-human) subjected to a treatment, observation or experiment. In some embodiments, the subject is a human having, or at risk of developing, Alzheimer's disease.

Therapeutic time window: The length of time during which an effective, or therapeutic dose, of a compound remains therapeutically effective in vivo.

Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

### II. COMPOUNDS AND MOLECULAR TARGETS FOR TREATING OR PREVENTING ALZHEIMER'S DISEASE (AD)

AD is characterized by well-known signs and symptoms. Signs and symptoms characteristic of AD include cognitive signs and symptoms (e.g., mental decline, difficulty thinking and/or understanding, delusion, disorientation, forgetfulness, mental confusion, difficulty concentrating, inability to create new memories, inability to perform simple math, inability to recognize common objects), behavioral signs and symptoms (e.g., aggression, agitation, difficulty with self-care, irritability, meaningless repetition of words, personality changes, restlessness, lack of restraint, wandering and getting lost), mood signs and symptoms (e.g., anger, apathy, general discontent, loneliness, mood swings), psychological signs and symptoms (e.g., depression, hallucination, paranoia), and other signs and symptoms (e.g., inability to combine muscle movements, jumbled speech, loss of appetite).

AD and the risk of developing AD also are characterized by certain biomarkers. For example, the risk of developing AD can be predicted by certain genetic markers. The ε4 allele of the apolipoprotein-E (APOE) gene is the most robust genetic risk factor for sporadic or late-onset AD. Heterozygous carriers of the ε4 allele are at 3-4 times greater risk of AD, while homozygous individuals are at 10 times greater risk relative to non-carriers (Farrer et al., JAMA 1997, 278:1349-1356; Neu et al., JAMA Neurol 2017, 74:1178-1189; Genin et al., Mol Psychiatry 2011, 16:903-907). Other biomarkers characteristic of AD include, but are not limited to, increased levels of extracellular amyloid beta (AB) concentration, tau phosphorylation, neuroinflammation, or any combination thereof.

In addition to the above biomarkers, a number of proteins exhibit altered expression in both AD and also in subjects at risk of developing AD but without significant AD pathology and/or symptoms. Based on the altered expression pattern of these proteins, the inventors have established an AD proteomic signature in subjects with AD as well as an incipient AD proteomic signature in young subjects, such as APOE ε4 carriers. The proteomic signatures are indicative of very early biological perturbations occurring during the long preclinical phase of AD and may present novel therapeutic molecular targets for disease modification.

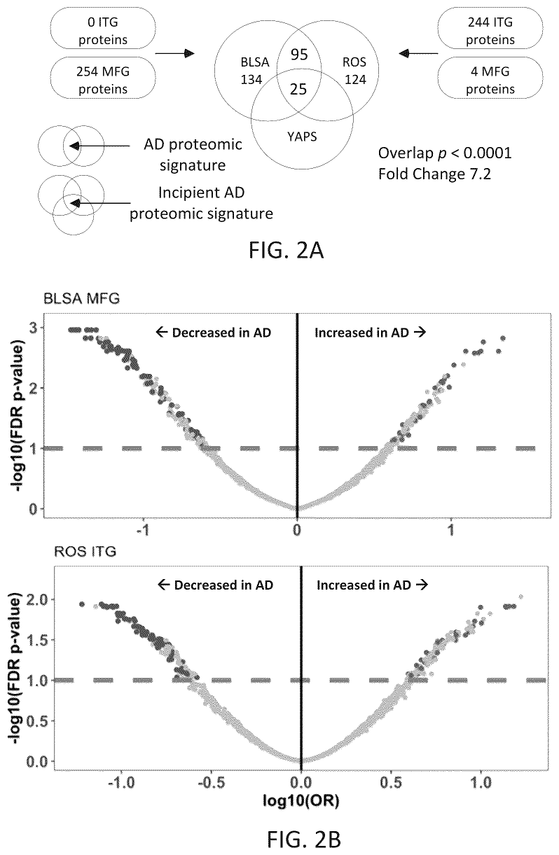

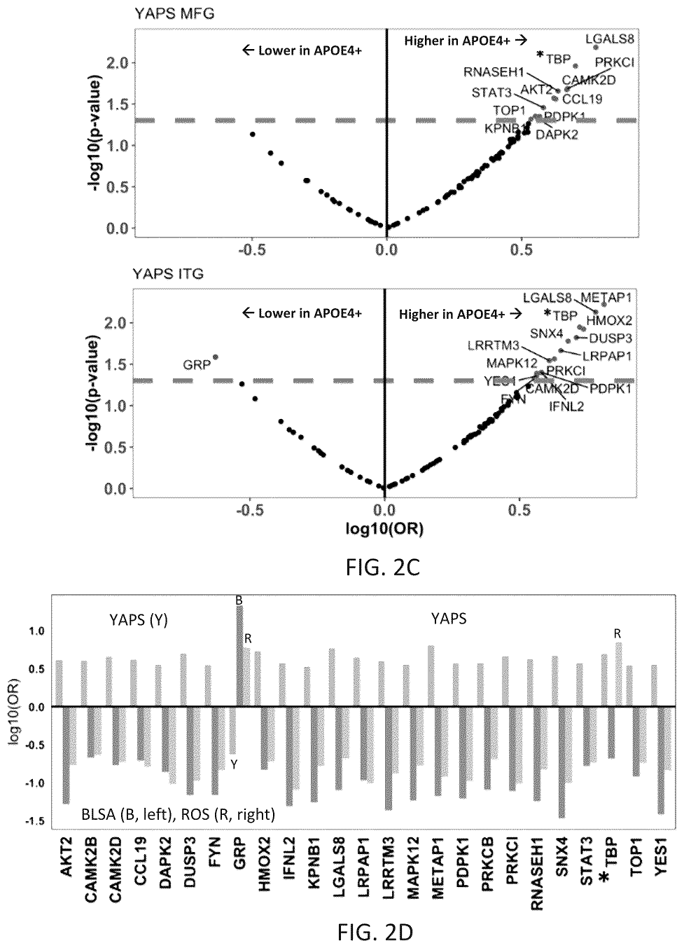

Intriguingly, as discussed in detail in Example 1 and shown in FIG. 2D, nearly all proteins in the proteomic signature show opposite aberrations between young APOE ε4 carriers and older AD individuals. Most proteins in the incipient proteomic signature are elevated in APOE ε4 carriers relative to non-carriers and are reduced in AD subjects relative to control subjects. Thus, alterations in brain proteomic profiles in young APOE ε4 carriers may represent early pathogenic changes in individuals at enhanced risk for AD. Over the next several decades, the functional consequences of these early molecular changes may cause the progressive accumulation of pathology, eventually manifesting as the irreversible cognitive impairment and functional decline characterizing the clinical syndrome of AD.

Protein biomarkers in the proteomic signature include AKT2, CAMK2B, CAMK2D, CCL19, DAPK2, DUSP3, FYN, GRP, HMOX2, IFNL2, KPNB1, LGALS8, LRPAP1, LRRTM3, MAPK12, METAP1, PDPK1, P4KCB, PRKCI, RNASEH1, SNX4, STAT3, TBP, TOP1, and YES1. A subject with AD or at risk of developing AD may have an aberrant level of one or more of these proteins. In some embodiments, the subject has an aberrant level of one or more proteins selected from the group consisting of DUSP3, FYN, LGALS8, STAT3, TOP1, and YES1.

These proteins are potential targets for disease modification. For instance, STAT3, YES1, and FYN modulate neuroinflammation, tau phosphorylation, and amyloid beta (Aβ) levels. In some embodiments, administering an active agent targeting one or more of the proteins in the proteomic signature may reduce levels of neuroinflammation, tau phosphorylation, and/or extracellular Aβ levels. In some embodiments, administering the active agent may modify development and/or progression of AD.

In some embodiments, a method includes administering to a subject an amount of an active agent effective to at least partially normalize an aberrant level of one or more indicators wherein the indicators (or phenotypes) comprise, or consist of, extracellular Aβ concentration, tau phosphorylation, neuroinflammation, plasma neurofilament light chain protein (NfL) concentration, hippocampal synaptic plasticity, or any combination thereof. Impaired hippocampal synaptic plasticity is a well-established neurobiological surrogate of learning and memory. These indicators are associated with Alzheimer's pathology. Normalizing the aberrant level of the one or more indicators may reduce extracellular Aβ concentration, decrease tau phosphorylation, reduce neuroinflammation, reduce plasma NfL concentration, increase hippocampal synaptic plasticity, or any combination thereof. In some embodiments, the active agent is effective to at least partially normalize aberrant levels of at least two of the indicators. For example, the active agent may at least partially normalize levels of both neuroinflammation and Aβ concentration. In certain embodiments, the active agent at least partially normalizes aberrant levels of at least three indicators. Advantageously, an active agent that targets multiple pathologies (i.e., by at least partially normalizing levels of multiple indicators) may reduce or eliminate the need to treat the subject with multiple active agents or interventions, thereby facilitating treatment, facilitating subject compliance with the treatment regimen, and/or reducing likelihood of adverse side effects.

In one implementation, reducing neuroinflammation comprises reducing a concentration of one or more interleukins and/or inflammatory cytokines, such as interleukin-6 (IL-6), interleukin-1β (IL-1β), interleukin IL-12p170 (IL-12p170), interleukin-10 (IL-10), tumor necrosis factor alpha (TNF-α), or any combination thereof. In an independent implementation, reducing extracellular Aβ concentration comprises reducing Aβ secretion, increasing Aβ clearance, or both.

Suitable active agents include agents that rescue molecular phenotypes relevant to AD pathogenesis by altering expressed levels of one or more of the proteins associated with the AD proteomic signature and/or incipient AD proteomic signature. In some embodiments, the active agent modifies expressed levels of DUSP3, FYN, LGALS8, STAT3, TOP1, YES1, or any combination thereof. In certain implementations, the active agent modifies levels of STAT3, FYN, YES1, or any combination thereof.

Exemplary active agents include, but are not limited to, C188-9 (TTI-101, N-[4-hydroxy-3-(2-hydroxynaphthalen-1-yl)naphthalen-1-yl]-4-methoxybenzenesulfonamide), dasatinib, hydroxychloroquine (HCQ), MLS-0437605 (N-4-fluoro-1,3-benzothiazol-2-71)-5-(4-methoxyphenyl)-1,3,4-oxadiazol-2-amine), methyl-β-d-galactomalonyl phenyl esters, irinotecan, pyrimethamine, TTI-102, tauroursodeoxycholic acid (TUDCA), DIM/arundine, or any combination thereof. In some embodiments, the active agent comprises C188-9, dasatinib, HCQ, or any combination thereof. In any of the foregoing or following embodiments, the subject may not have previously received the active agent, e.g., for another indication.

Administering in combination can include administering (i) a single pharmaceutical composition comprising two or more active agents, or (ii) administering two or more pharmaceutical compositions, each pharmaceutical composition comprising at least one active agent. When administering two or more pharmaceutical compositions, the pharmaceutical compositions may be administered simultaneously or sequentially in any order. When administered sequentially, the pharmaceutical compositions preferably are administered such that therapeutic time windows of each of the active agents at least partially overlap. The pharmaceutical compositions may be administered by the same or different routes.

C188-9 and HCQ both target STAT3 and advantageously at least partially normalize the levels of extracellular Aβ concentration and tau phosphorylation, and reduce neuroinflammation. C188-9 and HCQ modify extracellular Aβ concentration levels by differing mechanisms. C188-9 reduces Aβ secretion, whereas HCQ increases Aβ clearance in microglial cells. HCQ reduces neuroinflammation by reducing levels of TNF-α, IL-6, IL-1β, IL-12p70, and IL-10. HCQ also at least partially normalizes plasma NfL concentration and/or hippocampal synaptic plasticity. C188-9 reduces neuroinflammation by reducing levels of IL-6 and IL-1β. Dasatinib at least partially normalizes the level of tau phosphorylation by targeting YES1 and FYN, members of the Src family of tyrosine kinases.

TUDCA, a bile acid, reduces Aβ levels by reducing Aβ secretion. In some embodiments, TUDCA is administered in combination with HCQ, C188-9, or both.

In some implementations, TUDCA is administered in combination with HCQ. The combination provides synergy with HCQ at least partially normalizing the levels of extracellular Aβ concentration by increasing Aβ clearance and TUDCA reducing Aβ secretion. HCQ also provides additional benefits, e.g., at least partially normalizing levels of tau phosphorylation and/or reducing neuroinflammation as previously described. In one aspect, HCQ and TUDCA are administered in a single pharmaceutical composition comprising both HCQ and TUDCA. For example, HCQ and TUDCA may be combined into an oral dosage form, such as a suspension, tablet, or capsule comprising both HCQ and TUDCA. In an independent aspect, HCQ and TUDCA are administered simultaneously or sequentially in separate pharmaceutical compositions, one pharmaceutical composition comprising HCQ and one pharmaceutical composition comprising TUDCA. For example, HCQ and TUDCA may be administered individually as oral dosage forms, such as suspensions, tablets, or capsules. In any of the foregoing or following aspects, HCQ may be administered as in the form of nanoparticles (nano-HCQ). In any of the foregoing or following aspects, nano-HCQ may be in the form of poly(L-lysine succinylated)-HCQ (PLS-HCQ).

DIM is the dimeric product of the natural product indole-3-carbinole. DIM reduces Aβ levels and neuroinflammation, such as LPS-induced neuroinflammation. DIM has been shown to inhibit oxidative stress-induced apoptosis in hippocampal neuronal cells (Lee et al., Antioxidants 2019, 9(1):3) and protect hippocampal cell cultures from ischemia-induced apoptosis and autophagy (Rzemieniec et al., Apoptosis: An International Journal on Programmed Cell Death, 2019, 24:435-452). DIM has been shown to target the RGS4 gene (regulator o G-protein signaling 4) and to bind to the aryl hydrocarbon receptor, product of the AHR gene (Rzemieniec et al., Apoptosis: An International Journal on Programmed Cell Death, 2019, 24:435-452).

In some implementations, DIM is administered in combination with HCQ. The combination provides synergy with both HCQ and DIM at least partially normalizing the levels of extracellular Aβ concentration by increasing Aβ clearance. Both HCQ and DIM also reduce neuroinflammation. In addition, HCQ also at least partially normalizes levels of tau phosphorylation. In one aspect, HCQ and DIM are administered in a single pharmaceutical composition comprising both HCQ and DIM. For example, HCQ and DIM may be combined into an oral dosage form, such as a suspension, tablet, or capsule comprising both HCQ and DIM. In an independent aspect, HCQ and DIM are administered simultaneously or sequentially in separate pharmaceutical compositions, one pharmaceutical composition comprising HCQ and one pharmaceutical composition comprising DIM. For example, HCQ and DIM may be administered individually as oral dosage forms, such as suspensions, tablets, or capsules. In any of the foregoing or following aspects, HCQ may be administered as in the form of nanoparticles (nano-HCQ). In any of the foregoing or following aspects, nano-HCQ may be in the form of poly(L-lysine succinylated)-HCQ (PLS-HCQ).

In some aspects, HCQ, TUDCA, and DIM are administered in combination. In one aspect, HCQ, TUDCA and DIM are administered in a single pharmaceutical composition comprising all three active agents. For example, HCQ, TUDCA, and DIM may be combined into an oral dosage form, such as a suspension, tablet, or capsule comprising all three active agents. In an independent aspect, HCQ, TUDCA, and DIM are administered simultaneously or sequentially in separate pharmaceutical compositions, one pharmaceutical composition comprising HCQ, one pharmaceutical composition comprising TUDCA, and one pharmaceutical composition comprising DIM. For example, HCQ, TUDCA, and DIM may be administered individually as oral dosage forms, such as suspensions, tablets, or capsules. Alternatively, HCQ, TUDCA, and DIM are administered simultaneously or sequentially in separate pharmaceutical compositions, where one pharmaceutical composition comprises two of the active agents, and another pharmaceutical composition comprises the third active agent. In any of the foregoing or following aspects, HCQ may be administered as in the form of nanoparticles (nano-HCQ). In any of the foregoing or following aspects, nano-HCQ may be in the form of poly(L-lysine succinylated)-HCQ (PLS-HCQ).

In any of the foregoing or following embodiments, the subject may be diagnosed as having AD prior to administering the active agent(s). In some embodiments, the subject may have an aberrant level of one or more indicators of Alzheimer's pathology in the brain, cerebrospinal fluid, or blood, such as increased levels of extracellular Aβ concentration, tau phosphorylation, neuroinflammation, and/or plasma NfL concentration, and/or impaired hippocampal synaptic plasticity. The indicators may be measured by any suitable means. In some embodiments, the indicators are measured by a positron emission tomography (PET) scan. Aβ concentration and phosphorylated tau levels also can be measured by cerebrospinal fluid and/or blood assays. NfL concentration may be measured by blood tests. Hippocampal synaptic plasticity may be measured with neuroimaging methods, such as functional magnetic resonance imaging (fMRI) to determine functional connectivity and positron emission tomography (PET) to measure synaptic vesicle protein 2A (SV2A). In any of the foregoing or following embodiments, the subject may be at risk of developing AD, and the method may further comprise identifying the subject as being at risk of developing AD prior to administering the active agent(s). In any of the foregoing or following embodiments, the subject may further be identified as not having previously received the active agent(s).

In some implementations, the subject is identified as having AD or being at risk of developing AD by identifying the subject as being an APOE ε4 carrier. In some embodiments, the subject is identified based on an aberrant level of one of more indicators in the brain, such as extracellular Aβ concentration, tau phosphorylation and/or accumulation, neuroinflammation, and/or hippocampal synaptic plasticity. In certain embodiments, the subject is identified based on an aberrant level of one or more indicators in cerebrospinal fluid and/or blood, such as extracellular Aβ concentration, tau phosphorylation, and/or plasma NfL concentration. The subject may be identified as being at risk of developing AD by identifying the subject as being an APOE ε4 carrier, as having an elevated level of the one or more indicators in the brain relative to a normal level of the one or more indicators in the brain, and/or as having an aberrant level of the one or more indicators in cerebrospinal fluid and/or blood assays in the absence of any cognitive, behavioral, mood, or psychological signs or symptoms of AD. Less commonly, the subject may be identified as having AD or being at risk of developing AD by measuring initial levels of one or more of the indicators, administering the active agent, and subsequently measuring a post-administration level of the indicator(s). In certain implementations, the subject is administered the active agent for an effective period of time prior to measuring the post-administration level. The effective period of time may be, for example, two weeks, one month, two months, three months, six months, or longer. A post-administration level of the indicator(s) that is less aberrant than the initial level may be indicative of the subject having AD or being at risk of developing AD since the subject may have derived benefit from receiving the active agent. In some implementations, the subject at risk of AD is administered an active agent comprising C188-9, dasatinib, HCQ, MLS-0437605, a methyl-β-d-galactomalonyl phenyl ester, irinotecan, pyrimethamine, TTI-102, TUDCA, or any combination thereof. In certain embodiments, the subject is administered C188-9, hydroxychloroquine, dasatinib, TUDCA, or any combination thereof. In some examples, the subject is administered a combination of hydroxychloroquine and TUDCA.

In any of the foregoing or following embodiments, it may be beneficial to monitor levels of the one or more indicators to assess severity and/or progression of the pathology, to assess treatment efficacy, or both. In some embodiments, the method further includes receiving data comprising an initial level of the one or more indicators prior to administering the active agent to the subject. For example, imaging evaluation, such as PET scans, may determine levels of extracellular Aβ concentration (e.g., as evidenced by extent of Aβ deposition), tau accumulation (e.g., accumulation of phosphorylated tau), and/or neuroinflammation. Extracellular Aβ concentration, tau phosphorylation, and/or plasma NfL concentration also may be determined by laboratory evaluation, such as cerebrospinal fluid and/or blood-based assays. In certain implementations, the method may further include receiving data comprising a post-administration level of the one or more indicators following administration of the active agent to the subject, and selecting an adjusted amount of the active agent for administration to the subject based at least in part on the post-administration level of the one or more indicators.

In any of the foregoing or following embodiments, the method may comprise administering the active agent to the subject prophylactically in the absence of any cognitive, behavioral, mood, or psychological signs or symptoms of AD. For example, the active agent may be administered prophylactically to a subject identified as being at risk of developing AD.

### III. PHARMACEUTICAL COMPOSITIONS AND DOSING

Another aspect of the disclosure includes pharmaceutical compositions prepared for administration to a subject and which include an effective amount of one or more of the active agents disclosed herein. The therapeutically effective amount of a disclosed active agent will depend on the route of administration and the physical characteristics of the subject being treated. Specific factors that can be taken into account include disease severity and stage, weight, diet and concurrent medications. The relationship of these factors to determining a therapeutically effective amount of the disclosed active agents is understood by those of skill in the art.

Pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, PA, 19th Edition (1995), describes compositions and formulations suitable for pharmaceutical delivery of the active agents herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some embodiments, the formulation may comprise a plurality of nanoparticles, the nanoparticles comprising the active agent.

Pharmaceutical compositions disclosed herein include those formed from pharmaceutically acceptable salts and/or solvates of the disclosed active agents. Pharmaceutically acceptable salts include those derived from pharmaceutically acceptable inorganic or organic bases and acids. Particular disclosed active agents may possess at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with a-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2- sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain active agents may include at least one acidic group that can form an acid-base salt with an inorganic or organic base. Examples of salts formed from inorganic bases include salts of the presently disclosed active agents with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. In addition, quaternary ammonium counterions also can be used.

Particular examples of suitable amine bases (and their corresponding ammonium ions) for use in the present active agents include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris- (2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

The pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes.

To formulate the pharmaceutical compositions, the active agent can be combined with various pharmaceutically acceptable additives, as well as a base or vehicle for dispersion of the active agent. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The active agent can be dispersed in a base or vehicle, which can include a hydrophilic compound having a capacity to disperse the active agent, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The active agent can be combined with the base or vehicle according to a variety of methods, and release of the active agent can be by diffusion, disintegration of the vehicle, or associated formation of water channels. In some circumstances, the active agent is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time. For example, HCQ may be administered in nanoparticulate form, such as in HCQ-loaded liposomes, polymer-based nanoparticles or micelles, lipid-based nanoparticles, nanoemulsions, and metallic nanoparticle conjugates. (Stevens et al., Molecules 2021, 26(1):175). Exemplary polymers include, but are not limited to, methoxy PEG-b-poly(L-lactic acid), poly(lactic-co-glycolic acid) (PLGA), poly(N-isopropylacrylamide-co-acrylic acid), and poly(L-lysine).

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the active agent can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the active agent can be brought about by including in the composition an agent which delays absorption, for example, monostearate salts and gelatin.

In certain embodiments, the active agent can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the active agent in the required amount in an appropriate solvent with one or a combination of ingredients enumerated herein, as required, followed by filtered sterilization. Generally, dispersions are prepared by incorporating the active agent into a sterile vehicle that contains a basic dispersion medium and the required other ingredients from those enumerated herein. In the case of sterile powders, methods of preparation include vacuum drying and freeze-drying which yields a powder of the active agent plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

The effective amount of the active agent will depend upon the severity of the disease and the general state of the subject's health. An effective amount is that which provides either subjective relief of one or more signs or symptoms or an objectively identifiable improvement as noted by the clinician or other qualified observer. In one embodiment, an effective amount is the amount necessary to at least partially normalize a level of one or more indicators associated with AD. The effective amount of the agents administered can vary depending upon the desired effects and the subject to be treated.

The actual dosage of the active agent will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the active agent for eliciting the desired response in the subject. Dosage regimens can be adjusted to provide an optimum prophylactic or therapeutic response. An effective amount is also one in which any toxic or detrimental side effects of the active agent is outweighed in clinical terms by therapeutically beneficial effects. A non-limiting range for an effective amount of an active agent within the methods and formulations of the disclosure is about 0.01 mg/kg body weight to about 20 mg/kg body weight, such as about 0.05 mg/kg to about 10 mg/kg body weight, about 0.2 mg/kg to about 10 mg/kg body weight, or about 1 mg/kg to about 10 mg/kg.

Dosage can be varied by the attending clinician as previously described, such as based on a determined level of the one or more indicators associated with AD. Higher or lower concentrations can be selected based on the mode of delivery, for example, trans-epidermal, rectal, oral, pulmonary, intraosseous, or intranasal delivery versus intravenous or subcutaneous or intramuscular delivery. Dosage can also be adjusted based on the release rate of the administered formulation, for example, of an intrapulmonary spray versus powder, sustained release oral versus injected particulate or transdermal delivery formulations, and so forth.

In some aspects, a HCQ dosage for an adult human may be from 50 mg to 1000 mg, such as from 200 mg to 500 mg, or from 0.5 mg/kg body weight to 20 mg/kg body weight, such as from 2 mg/kg body weight to 10 mg/kg body weight, daily in one dose or two or more divided doses. In some aspects, a TUDCA dosage for an adult human may be from 100 mg to 2000 mg, such as from 500 mg to 1000 mg, or from 1 mg/kg body weight to 40 mg/kg body weight daily, such as from 5 mg/kg body weight to 20 mg/kg body weight, daily in one dose or two or more divided doses. In some aspects, an DIM dosage for an adult human may be from 25 mg to 200 mg, such as from 100 mg to 150 mg, or from 0.25 mg/kg body weight to 4 mg/kg body weight, such as from 1 mg/kg body weight to 3 mg/kg body weight, daily in one dose or two or more divided doses.

For prophylactic and therapeutic purposes, the active agent can be administered to the subject by the oral route or in a single bolus delivery, via continuous delivery (for example, continuous intravenous delivery) over an extended time period, or in a repeated administration protocol (for example, by an hourly, daily or weekly, repeated administration protocol). The effective dosage of the active agent can be provided as repeated doses within a prolonged prophylaxis or treatment regimen that will yield clinically significant results to alleviate one or more symptoms associated with AD and/or at least partially normalize a level of one or more indicators associated with AD. Determination of effective dosages in this context is typically based on animal model studies followed up by human clinical trials and is guided by administration protocols that significantly reduce the occurrence or severity of AD symptoms or at least partially normalize the level of the one or more indicators in the subject. Suitable models in this regard include, for example, murine, rat, avian, dog, sheep, porcine, feline, non-human primate, and other accepted animal model subjects known in the art. Alternatively, effective dosages can be determined using in vitro models. Using such models, only ordinary calculations and adjustments are required to determine an appropriate concentration and dose to administer an effective amount of the active agent.

Treatment can involve daily or multi-daily doses of active agent(s) over a period of a few days to months, or even years. Thus, the dosage regimen will also, at least in part, be determined based on the particular needs of the subject to be treated and will be dependent upon the judgment of the administering practitioner. In particular examples, the subject is administered a therapeutic composition that includes one or more of the disclosed active agents on a multiple daily dosing schedule, such as at least two consecutive days, 10 consecutive days, and so forth, for example for a period of weeks, months, or years. In one example, the subject is administered the composition for a period of at least 30 days, such as at least 2 months, at least 4 months, at least 6 months, at least 12 months, at least 24 months, at least 36 months, at least 5 years, at least 10 years, or indefinitely for the remainder of the subject's life.

In some embodiments, the subject may further be administered additional therapeutic agents. For example, the subject may be administered one or more additional therapeutic agents used for treating AD or for treating one or more signs or symptoms associated with AD. Preparation and dosing schedules for the additional agent may be used according to manufacturer's instructions or as determined empirically by the skilled practitioner. The combination therapy may provide synergy and prove synergistic, that is, the effect achieved when the active agent and therapeutic agent used together is greater than the sum of the effects that results from using the active agent and therapeutic agent separately. A synergistic effect may be attained when the active agent and additional therapeutic agent are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the active agent and therapeutic agent are administered or delivered sequentially, for example by different injections in separate syringes. In general, during alternation, an effective dosage of the active agent and of the therapeutic agent is administered sequentially, i.e. serially, whereas in combination therapy, effective dosages of the active agent and therapeutic agent are administered together.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the active agents described herein. In one example, this component is formulated in a pharmaceutical preparation for delivery to a subject. The active agent is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

### IV. REPRESENTATIVE ASPECTS

Certain representative aspects are exemplified in the following numbered paragraphs.

1. A method, comprising administering to a subject an amount of an active agent effective to at least partially normalize an aberrant level of one or more indicators in the brain, wherein the indicators comprise extracellular amyloid beta (Aβ) concentration, tau phosphorylation, neuroinflammation, or any combination thereof.

2. The method of paragraph 1, wherein normalizing the aberrant level of the one or more indicators in the brain reduces extracellular Aβ concentration, decreases tau phosphorylation, reduces neuroinflammation, or any combination thereof.

3. The method of paragraph 2, wherein reducing extracellular Aβ concentration in the brain comprises reducing Aβ secretion, increasing Aβ clearance, or both.

4. The method of paragraph 2, wherein reducing neuroinflammation comprises reducing a concentration of interleukin-6, interleukin-1β, interleukin-12p70, interleukin-10, tumor necrosis factor alpha, or any combination thereof.

5. The method of any one of paragraphs 1-4, wherein the active agent comprises C188-9 (TTI-101, N-[4-hydroxy-3-(2-hydroxynaphthalen-1-yl)naphthalen-1-yl]-4-methoxybenzenesulfonamide), dasatinib, hydroxychloroquine, MLS-0437605 (N-4-fluoro-1,3-benzothiazol-2-71)-5-(4-methoxyphenyl)-1,3,4-oxadiazol-2-amine), a methyl-β-d-galactomalonyl phenyl ester, irinotecan, pyrimethamine, TTI-102, or any combination thereof.

6. The method of paragraph 5, wherein the active agent comprises C188-9, dasatinib, hydroxychloroquine, or any combination thereof.

7. The method of any one of paragraphs 1-6, wherein the active agent at least partially normalizes the level of at least two of the one or more indicators in the brain.

8. The method of paragraph 7, wherein the active agent reduces neuroinflammation and extracellular Aβ concentration in the brain.

9. The method of paragraph 7 or paragraph 8, wherein the active agent comprises C188-9, hydroxychloroquine, or a combination thereof.

10. The method of any one of paragraphs 1-6, wherein the active agent at least partially normalizes the level of extracellular Aβ concentration, tau phosphorylation, and neuroinflammation in the brain.

11. The method of paragraph 10, wherein the active agent comprises C188-9, hydroxychloroquine, or a combination thereof.

12. The method of any one of paragraphs 1, 2, 5, or 6, wherein the active agent inhibits tau phosphorylation in the brain.

13. The method of paragraph 12, wherein the active agent comprises dasatinib, C188-9, hydroxychloroquine, or a combination thereof.

14. A method, comprising administering to a subject an amount of an active agent effective to at least partially normalize aberrant levels of two or more indicators in the brain, wherein the indicators comprise extracellular amyloid beta (Aβ) concentration, tau phosphorylation, neuroinflammation, or any combination thereof.

15. A method, comprising administering to a subject an amount of an active agent effective to at least partially normalize aberrant levels of three indicators in the brain, wherein the indicators comprise extracellular amyloid beta (Aβ) concentration, tau phosphorylation, and neuroinflammation.

16. The method of paragraph 14 or paragraph 15, wherein the active agent comprises C188-9, hydroxychloroquine, or a combination thereof.

17. The method of any one of paragraphs 1-16, further comprising receiving data comprising an initial level of at least one of the indicators in the brain prior to administering the active agent to the subject.

18. The method of any one of paragraphs 1-17, further comprising: receiving data comprising a post-administration level of at least one of the indicators in the brain following administration of the active agent to the subject; and selecting an adjusted amount of the active agent for administration to the subject based at least in part on the post-administration level.

19. The method of any one of paragraphs 1-18, wherein the subject is diagnosed as having Alzheimer's disease (AD) prior to administering the active agent.

20. The method of any one of paragraphs 1-15, further comprising, prior to administering to the subject the active agent, identifying the subject as being at risk of developing AD by: (i) identifying the subject as being an APOE ε4 carrier; or (ii) identifying the subject as having an elevated level of the one or more indicators in the brain relative to a normal level of the one or more indicators in the brain; or (iii) both (i) and (ii).

21. The method of any one of paragraphs 1-18 or 20, further comprising administering the active agent to the subject prophylactically in the absence of any cognitive, behavioral, mood, or psychological signs or symptoms of AD.

22. A method for inhibiting progression of AD, comprising administering to a subject diagnosed as having AD an amount of an active agent effective to at least partially normalize an aberrant level of one or more indicators of Alzheimer's disease pathology in the brain wherein the indicators comprise extracellular amyloid beta (Aβ) concentration, tau phosphorylation, neuroinflammation in the brain, or any combination thereof.

23. The method of paragraph 22, wherein the active agent comprises C188-9, dasatinib,

hydroxychloroquine, MLS-0437605, a methyl-β-d-galactomalonyl phenyl ester, irinotecan, pyrimethamine, TTI-102, or any combination thereof.

24. A method for inhibiting or preventing development of AD, comprising: identifying a subject as being at risk of developing AD; and administering to the subject at risk of developing AD an amount of an active agent effective to at least partially normalize an aberrant level of one or more indicators of Alzheimer's disease pathology in the brain, wherein the indicators comprise extracellular amyloid beta (Aβ) concentration, tau phosphorylation, neuroinflammation in the brain, or any combination thereof.

25. The method of paragraph 24, wherein the active agent comprises C188-9, dasatinib, hydroxychloroquine, MLS-0437605, a methyl-β-d-galactomalonyl phenyl ester, irinotecan, pyrimethamine, TTI-102, or any combination thereof.

26. The method of paragraph 25 or paragraph 25, wherein the subject is identified as being at risk of developing AD by identifying the subject as: (i) being an APOE ε4 carrier; or (ii) having an elevated level of the one or more indicators in the brain relative to a normal level of the one or more indicators in the brain in the absence of any cognitive, behavioral, mood, or psychological signs or symptoms of AD; or (iii) both (i) and (ii).

27. An active agent for use in a method of at least partially normalizing an aberrant level of one or more indicators in the brain, wherein the indicators comprise extracellular amyloid beta (Aβ) concentration, tau phosphorylation, neuroinflammation, or any combination thereof, the method comprising administering to a subject an amount of the active agent effective to at least partially normalize the aberrant level of the one or more indicators.

28. An active agent for use in a method of treating AD, the method comprising administering to a subject diagnosed as having AD, an amount of an active agent effective to at least partially normalize an aberrant level of one or more indicators of AD pathology in the brain, wherein the indicators comprise extracellular amyloid beta (Aβ) concentration, tau phosphorylation, neuroinflammation, or any combination thereof.

29. The active agent for use in the method of paragraph 27 or paragraph 28, wherein the active agent comprises C188-9, dasatinib, hydroxychloroquine, MLS-0437605, a methyl-β-d-galactomalonyl phenyl ester, irinotecan, pyrimethamine, TTI-102, or any combination thereof.

30. Use of an active agent for at least partially normalizing an aberrant level of one or more indicators of AD pathology in the brain in a subject diagnosed as having AD, wherein the active agent comprises C188-9, dasatinib, hydroxychloroquine, MLS-0437605, a methyl-β-d-galactomalonyl phenyl ester, irinotecan, pyrimethamine, TTI-102, or any combination thereof, wherein the indicators comprise extracellular amyloid beta (Aβ) concentration, tau phosphorylation, neuroinflammation, or any combination thereof.

31. Use of an active agent for the treatment of AD, wherein the active agent comprises C188-9, dasatinib, hydroxychloroquine, MLS-0437605, a methyl-β-d-galactomalonyl phenyl ester, irinotecan, pyrimethamine, TTI-102, or any combination thereof

32. An active agent for use in a method of inhibiting or preventing development of AD, the method comprising administering to a subject identified as being at risk of AD an amount of an active agent effective to at least partially normalize an aberrant level of one or more indicators of AD pathology in the brain, wherein the indicators comprise extracellular amyloid beta (Aβ) concentration, tau phosphorylation, neuroinflammation, or any combination thereof, and wherein the active agent comprises C188-9, dasatinib, hydroxychloroquine, MLS-0437605, a methyl-β-d-galactomalonyl phenyl ester, irinotecan, pyrimethamine, TTI-102, or any combination thereof.

33. The active agent for use in the method of paragraph 32, wherein the subject is

identified as being at risk of developing AD on the basis of: (i) being an APOE ε4 carrier; or (ii) having an elevated level of the one or more indicators in the brain relative to a normal level of the one or more indicators in the brain in the absence of any cognitive, behavioral, mood, or psychological signs or symptoms of AD; or (iii) both (i) and (ii).

34. Use of an active agent for inhibiting or preventing development of AD in a subject identified as being at risk of developing AD, wherein the active agent comprises C188-9, dasatinib, hydroxychloroquine, MLS-0437605, a methyl-β-d-galactomalonyl phenyl ester, irinotecan, pyrimethamine, TTI-102, or any combination thereof.

### V. EXAMPLES

### Examples 1-3

### Materials and Methods

### Subject Details

Baltimore Longitudinal Study of Aging (BLSA): The National Institute on Aging's (NIA) BLSA is among the longest running scientific studies of aging in the U.S. (Ferrucci et al., J Gerontol A Biol Sci Med Sci 2008, 63:1416-1419). This observational study began in 1958 and includes longitudinal, radiological, clinical, and laboratory evaluations of community-dwelling volunteer participants. The individuals included in this study were participants in the autopsy sub-study of the BLSA, which has been described previously (O'Brien et al., J Alzheimers Dis 2009, 18:665-675). Postmortem brains were examined by an expert neuropathologist to assess AD pathology. The Consortium to Establish a Registry for Alzheimer's Disease (CERAD) and Braak criteria were used to assess severity of AD pathology based on neuritic plaques (Mirra et al., Neurology 1991, 41:479-486) and neurofibrillary tangles (braak et al., Acta Neuropathologica 1991, 82:239-259) respectively, as described previously Troncoso et al., Annals of Neurology 2008, 64:168-176). Clinical diagnosis of dementia and AD have previously been described (Kawas et al., Neurology 2000, 64:2072-2077) and were based on the Diagnostic and Statistical Manual (DSM)-III-R (APA, Diagnostic and statistical manual of mental disorders: DSM-III-R. (American Psychiatric Association, Washington, DC, 1987)) and the National Institute of Neurological and Communication Disorders and Stroke-Alzheimer's Disease and Related Disorders Association (NINCDS-ADRDA) criteria, respectively (McKhann et al., Neurology 1984, 34:939-944).

Autopsy participants were classified as either AD or CN according to the following criteria: AD participants (n=31) had a clinical diagnosis of AD or mild cognitive impairment (MCI) within 1 year of death in addition to a post-mortem CERAD pathology score>1 (i.e., CERAD B or C); cognitively normal (CN) participants (n=19) had normal cognition within 1 year of death and a CERAD pathology score≤1 (i.e., CERAD 0 or A). Diagnosis and cognitive status were determined at consensus diagnosis conferences using procedures described in detail previously Kawas et al., Neurology 2000, 64:2072-2077). Demographic characteristics of the BLSA cohort are included in Table 1 (Example 1). The BLSA study protocol has ongoing approval from the Institutional Review Board of the National Institute of Environmental Health Science, National Institutes of Health.

Religious Orders Study (ROS): The Religious Orders Study (ROS) has enrolled Catholic nuns, priests, and brothers from a multitude of communities across the U.S since 1994 (Bennett et al., J Alzheimers Dis 2018, 64:S161-S189). This longitudinal observational study collects information from clinical, neuroimaging, laboratory, and self-report evaluations of employed and retired community-dwelling individuals. At the time of enrollment, participants did not have a diagnosis of known dementia. All participants agreed to organ donation and annual clinical evaluation.

The sample consisted of a subset of participants from the larger ROS cohort study. All ROS participants provided written informed consent and the study was approved by an Institutional Review Board of Rush University Medical Center. Participants signed an Anatomical Gift Act for organ donation and a repository consent to allow their data and biospecimens to be shared. At each study visit, dementia status was determined by trained clinicians using all cognitive and clinical data blinded to prior years based upon NINCDS-ADRDA criteria. A final consensus clinical diagnosis was determined at death blinded to all neuropathologic data. Autopsies were performed based on standard methods reported previously (Schneider et al., Neurology 2003, 60:1082-1088). Postmortem brains were examined by an expert neuropathologist or trained technician to assess AD pathology. CERAD and Braak criteria were used to assess severity of AD pathology, as described previously (Bennett et al., Neurology 2003, 60:246-252).

Participants were classified into two groups. AD participants (n=31) had a final clinical diagnosis of AD and an NIA-Reagan score of intermediate or high likelihood of AD. NIA-Reagan criteria are based on both neuritic plaques (CERAD score) and neurofibrillary tangles (Braak score) (Neurobiology of Aging 1997, 18:S1-2). CN participants (n=22) had a clinical diagnosis of no cognitive impairment (NCI) and an NIA-Reagan score of low likelihood of AD or no AD. Diagnosis and cognitive status were determined based on a three-stage process described previously (Bennett et al., J Alzheimers Dis 2018, 64:S161-S189). Demographics of the ROS autopsy cohort are included in Table 1 (Example 1).

Young APOE Postmortem Study (YAPS): The Young APOE Postmortem Study (YAPS) was composed of postmortem brain tissue samples acquired from the Brain Resource Center at the Johns Hopkins Alzheimer's Disease Research Center (ADRC). All autopsies were performed at the Office of the Chief Medical Examiner of the State of Maryland in Baltimore. Study of postmortem samples was conducted under a protocol authorized by the Johns Hopkins University Institutional Review Board. The clinical and cognitive status of these subjects were undetermined. Brain tissue samples from these participants had CERAD and Braak scores of 0, indicating absence of significant AD pathology. See Pletnikova et al. for additional details on the study sample (Neurobiol Aging 2018, 71:72-80). YAPS samples included in this study are a convenience sample designed to include approximately 50 percent APOE ε4+ individuals (n=18 ε4+; n=17 ε4−). APOE genotyping was conducted on frozen tissue using the methods of Hixon and Vernier (J Lipid Res 1990, 31:545-548). Demographic characteristics of the YAPS cohort are included in Table 1 (Example 1). The age of individuals included in the YAPS cohort precedes the typical age of onset of AD by approximately 3 decades (Huff et al., J Am Geriatr Soc 1987, 35:27-30).

### Aptamer-Based Proteomics

Brain Tissue Collection and Homogenization: YAPS, BLSA, and ROS brain tissue samples were selected from two a priori specified regions: the inferior temporal gyrus (ITG) and middle frontal gyrus (MFG), which are susceptible to accumulation of classical AD neuropathology. All brain samples were stored at −80° C. For sample extraction, brain samples were placed in −20° C. freezer for 15 minutes and then sterile, 4-mm-diameter tissue punches were used to extract samples from the cortical surface of the brain tissue regions. Samples were again stored at −80° C. prior to proteomic assays.

To ˜10 mg of brain tissue, 110 μl of T-PER (tissue protein extraction reagent) (Thermo Fisher Scientific, USA) with 2 μl of Halt™ Protease and Phosphatase Inhibitor cocktail (Thermo Fisher Scientific, USA) was added and placed in a CKMix grinding tube, containing soft tissue homogenizing lysis beads (a mix of 1.4 mm and 2.8 mm ceramic (zirconium oxide) beads) (Bertin Technologies, San Quentin, France). The tubes were placed in the Precellys Evolution tissue homogenizer (Bertin Technologies, San Quentin, France) and homogenized for two 30 second cycles of 6500 rpm and a 30 second rest in between. The homogenate was removed and placed in an Eppendorf tube and centrifuged at 16,000×g for 5 minutes at 4° C. The supernatant was removed and centrifuged a second time for 10 minutes at 16,000×g at 4° C. The supernatant was collected at 4° C., 2.5 μl was aliquoted and protein quantitation was carried out using a MicroBCA protein Assay Kit (Thermo Scientific, USA). The total protein concentration was determined, and the samples were diluted to a final volume of 200 μg/ml with PBS 1× and stored at −80° C. until analysis.

Proteomic Quantification: Sample total protein was adjusted to 16 μg/mL in SB17T buffer (40 mM HEPES, 125 mM NaCl, 5 mM KCl, 5 mM MgCl₂, 1 mM EDTA, 0.05% Tween-20 at pH 7.5). Proteomic profiles for 1,322 SOMAmers were assessed using the 1.3K SOMAscan assay at the Trans-NIH Center for Human Immunology and Autoimmunity, and Inflammation (CHI), National Institute of Allergy and Infectious Disease, National Institutes of Health (Bethesda, MD, USA). The SOMAscan assay platform includes 1322 SOMAmer Reagents, of which 12 are hybridization controls, 5 are viral proteins, and 5 are non-specifically-targeted SOMAmers. As a result, analyses included 1,300 SOMAmer Reagents. The experimental procedure for proteomic assessment and normalization has been previously reported (Candia et al., Sci Rep 2017, 7:14248). In brief, targets were generated by a process known as Selected Evolution of Ligands by Exponential Enrichment (SELEX), a method of identifying high-affinity binding targets from much larger sequence libraries. This method allows the accurate detection of proteins spanning a dynamic range of 8 orders of magnitude (Gold et al., Plos One 2010, 5:e15004). The SOMAscan platform has been used widely for proteomic quantification in the context of multiple diseases and tissues allowing for replication and validation of findings.

The SOMAscan assay uses SOMAmers to translate protein concentrations into measurable DNA signals which can be quantified using standard DNA detection procedures. This is achieved by affinity binding and biotin capture on streptavidin beads. The DNA concentrations obtained from this method are reported as relative fluorescence units (RFUs), resulting from fluorescent SOMAmer hybridized to its complimentary probe on an Agilent array, and are directly proportional to the reported relative abundance of SOMAmer Reagents. Study/cohort specific samples were run in the same batch on separate plates. Within study/cohort, samples were randomized by disease (AD, CN), brain region (ITG, MFG), sex and age. The data normalization process across all plates and cohorts includes hybridization, control normalization, median signal normalization, and calibration normalization, as previously described (Candia et al., Sci Rep 2017, 7:14248). As an independent quality check (QC) for each analyte, we determined the coefficient of variation (CV; median 13-15%) obtained from 4 pairs of technical duplicates according to 3 different methods (Hyslop, J Air Waste Manag Associ 2009, 59:1032-1039; Bland et al., BMJ 1996, 313:106; Jones et al., Clinical investigation and statistics in laboratory medicine, ACB Venture Publications, London, 1997). For more details on the assay's performance, see Candia et al. (Sci Rep 2017, 7:14248).

Measurement Range: The Relative Fluorescence Unit (RFU) measurement range across all tissue samples and analytes spanned almost 5 orders of magnitude, from 49 to 2.6×10⁶.

Reproducibility/Replicates: By vendor's specifications, SOMAscan's plate design does not require technical replicates. Nonetheless, as an independent quality check (QC) for each analyte, we determined the coefficient of variation (CV) obtained from 4 pairs of technical duplicates according to 3 different methods (Hyslop et al., J Air Waste Manag Associ 2009, 59:1032-1039; Bland et al., BMJ 1996, 313:106; Jones et al., Clinical investigation and statistics in laboratory medicine, ACB Venture Publications, London, 1997), which are provided in Table 4 (Example 2). The CVs (median 13-15% were higher than those estimated by SomaLogic as well as by other independent studies(median 3-4%). These differences were likely driven by the small number of replicates used in this study (4 pairs). For more details on the assay's performance, see Candia et al, (Sci Rep 2017, 7:14248). A web-based interactive resource with CVs obtained from the analysis of a large data repository is available at https://foocheung.shinyapps.io/SOMACV3.

### Statistical Analyses

Demographic Characteristics: Demographic characteristics of the three cohorts (YAPS, BLSA, ROS) are summarized in Table 1 (Example 1). Comparisons between BLSA and ROS cohorts were performed using two-sample t-tests for continuous variables and chi-square tests for independence for categorical variables.

Incipient AD Signature: Definition and Analysis: A multi-step process was utilized to identify proteins significantly altered in all 3 primary cohorts in this study (i.e., BLSA, ROS, and YAPS). In Step 1, the AD proteomic signature was defined as the proteins differentially abundant between AD and CN in both BLSA (n_{AD}=31; n_{CN}=19) and ROS (n_{AD}=31; n_{CN}=22), i.e. shared proteins in two independent cohorts, in either the ITG or MFG. Separate proportional odds models (An et al., Alzheimers Dement 2018, 14:318-329) were used, a generalization of the non-parametric Wilcoxon and Kruskal-Wallis tests, for each protein in both the ITG and MFG brain regions. All models included ranked protein levels (outcome), the group predictor (AD vs CN) and covariates -sex, and age at death. Statistical significance of differentially abundant proteins included in the AD proteomic signature was corrected for multiple comparisons using a Benjamini-Hochberg false-discovery rate (Benjamini et al., J R Stat Soc B 1995, 57:289-300) (FDR) adjusted p-value <0.10 in both BLSA and ROS.

In Step 2, it was determined whether proteins included in the AD proteomic signature were also significantly different between young APOE ε4 carriers (n=18) and non-carriers (n=17) in either the ITG or MFG in the YAPS cohort. Participants were indicated as APOE ε4+ if they had at least one ε4 allele; participants were indicated as APOE ε4− if they did not carry the ε4 allele. Separate proportional odds models were performed as above, including the ranked protein levels (outcome), the group predictor (ε4+ vs ε4−), and covariates—sex, race (white vs non-white), and age at death. Because analyses were restricted only to those proteins identified as dysregulated in the AD proteomic signature, the significance threshold was set as p<0.05 for identifying proteins differing in the YAPS cohort. The incipient AD signature was defined as the proteins in the AD proteomic signature that were also altered in ε4 carriers versus non-carriers in the YAPS cohort.

Controlling for Type I Error: In STAGE 1: DISCOVERY, a set of proteins of interest was derived by identifying proteins which differed significantly between conditions in each of the three discovery cohorts. The issue of potential Type I errors was addressed in two ways. First - in the first step of the discovery stage where the AD proteomic signature was defined, multiple comparisons were corrected for using an FDR threshold. In the second step of the discovery stage where the overlap between the AD proteomic signature and proteins differentially expressed by APOE carriers in YAPS (i.e., the incipient AD signature) was defined, the statistical distributions of the multi-set intersection were computed and their exact probabilities were calculated to determine the statistical significance of the intersection between YAPS, BLSA and ROS using the SuperExactTest package in R (Wang et al., Sci Rep2015, 5:16923). This method determines the number of intersecting proteins that would be expected across the 3 cohort signatures if the proteins were picked at random and compares the null ‘expected intersection’ to the ‘observed intersection’ to generate a p-value and fold-enrichment (i.e. observed intersection size/expected intersection size).

Second, a rigorous two-step validation approach was applied in STAGE 2: VALIDATION to confirm the index results (i.e., the incipient AD proteomic signature). First, in Step 6a: Primary Validation, a subset of 6 proteins from the incipient AD signature was selected for validation based on their plausibility as targets of approved and experimental drugs in other diseases. These proteins were validated using immunoblotting of brain homogenates from the 3xTg-AD transgenic mouse model of AD. Next in Step 6b: Secondary Validation, the incipient AD signature was validated in three, independent publicly available datasets using orthogonal methods, including: 1) Two-Dimensional Liquid Chromatography-Tandem Mass Spectrometry (LC/LC-MS/MS) based proteomics in the Mount Sinai brain bank and a 5xFAD transgenic mouse model of AD; and 2) single-cell transcriptomics (i.e., single-cell RNA sequence (scRNA-Seq)) from the ROSMAP cohort. These validation studies are described in detail below in the section STAGE 2: VALIDATION of the incipient AD signature.

Pathway Enrichment and Protein-Protein Interaction Analysis: To determine if proteins in the incipient AD proteomic signature were functionally related, pathway enrichment analysis was performed using the MSigDB database (https://www.gsea-msigdb.org/gsea/msigdb/) (Subramanian et al., PNAS USA 2005, 102:15545-15550), as well as protein-protein interaction analyses using StringDB (https://string-db.org) (Szklarczyk et al., Nucleic Acids Res 2019, 47:D607-D613).

StringDB utilizes numerous publicly available sources to provide both physical and functional interactions between proteins, further visualizing results as a nodal interaction network. The 25 proteins from the incipient signature were input into the StringDB database and the internode interactions resulting from the program's computational predictions were recorded.

Associations with AD Pathology: Step 3 evaluated whether brain tissue protein levels in the incipient AD proteomic signature were associated with severity of AD pathology in BLSA and ROS within the ITG and MFG. Similar to prior studies (Varma et al., Plos Med 2018, 15(1):e1002482), partial Spearman correlations of CERAD and Braak scores with ranked aptamer values were examined, controlling for covariates—mean-centered sex and age at death. A significant (p<0.05) positive correlation indicated that higher concentration of the protein was associated with higher AD pathology (i.e., higher CERAD or Braak scores) and a significant (p<0.05) negative correlation indicated that lower concentration of the protein was associated with higher AD pathology.

Associations with Longitudinal Cognitive Performance: Step 4 evaluated whether brain tissue protein levels in the incipient AD proteomic signature were associated with ante-mortem trajectories of cognitive performance among individuals with AD in BLSA and ROS. Similar to prior studies (an et al., Alzheimers Dement 2018, 14:318-329), linear mixed effects models were used to determine whether brain tissue protein levels at death were associated with longitudinal changes in cognitive performance, specifically the mini mental state exam (MMSE), prior to death. MMSE scores at each visit were used as the outcome variable. Predictors included protein, sex, age at death, time (time to the last visit), protein*time, age at death*time, and sex*time. Random effects included a random intercept. The origin of time variable was anchored to the last visit. The coefficient of interest was protein*time: a significant (p<0.05) positive coefficient indicated that higher concentration of the protein was associated with slower/reduced decline in MMSE over time; a significant (p<0.05) negative coefficient indicated that increased concentration of the protein was associated with faster increased decline in MMSE over time. Similar to prior analyses (Roberts et al., Int J Mol Sci 2020, 21(4): 1249), protein concentration (predictor) was centered at the median, rescaled using the interquartile range (IQR), and outliers greater than 3*IQR were excluded. Age of death was mean-centered and sex was coded 0 as male, 1 as female.

Gene Set Enrichment Analysis: In Step 5, gene set enrichment analysis (GSEA) was used to identify the AD-related biologic pathways that may be altered in young APOE ε4 individuals by comparing enriched gene sets in the young sample to the older adult sample. GSEA offers an important complement to analyses of differences in abundance of individual proteins by determining the extent to which biologically-defined collections of genes are impacted as a group under a given biological context (Subramanian et al., PNAS USA 2005, 102:15545-15550). GSEA captures groups of proteins that share common biologic functions that may be different between AD and CN despite non-significant differences in single-protein analyses. GSEA was performed in R using the fgsea package (Sergushichev, bioRxiv 2016) on all 1300 proteins included in our dataset and selected gene sets from the Molecular Signatures Database (v6.0 MsigDB). Exploratory GSEA analysis was conducted using the following gene sets from MsigDB: 4 from the Blalock et al. AD gene sets (78), 289 from BioCarta, 186 from Kyoto Encyclopedia of Genes and Genomes (KEGG), 1499 from Reactome, and 7350 from Gene Ontology (GO) Biological Processes. Gene sets with <10 and >300 genes were excluded, resulting in 2406 gene sets utilized in GSEA. In these analyses, significance was set as a false discovery rate (FDR)-corrected p-value<0.05. Proteins were ranked for GSEA based on the odds ratio (OR) calculated by the proportional odds models described in Step 1. In GSEA analyses, magnitude of enrichment of gene sets was quantified using the normalized enrichment score (NES). The NES represents a weighted Kolmogorov-Smirnov test statistic and corresponds to the extent to which a specific gene set is overrepresented at the top or bottom extremes of the ranked protein list. A positive NES represents over-expression of a gene set, while a negative NES represents under-expression.

In order to determine the statistical significance of the overlap between AD and YAPS enriched gene sets, the significance of this overlap was tested by performing Fisher's exact ratio tests between the number of overlapping gene sets at FDR thresholds of 0.05, 0.1 and 0.25.

Sensitivity Analyses: In order to test whether proteomic differences between AD and CN in the older cohorts were driven predominantly by APOE ε4 individuals, all APOE ε4+ individuals from ROS and BLSA were excluded and proportional odds models identical to the main analysis were performed. It was then determined whether proteins included in this non-APOE ε4+ AD proteomic signature differed between APOE ε4 carriers and non-carriers in the YAPS cohort as in the main analysis.

Similarly, additional sensitivity analyses were performed in which the sample was restricted to only APOE ε4 carriers in ROS and BLSA and AD and CN individuals were compared. Owing to small sample size, proportional odds models similar to the main analysis without covariates were performed and only we performed proteins included in the incipient AD proteomic signature were tested.

### Stage 2: Validation of the Incipient AD Signature

To validate the incipient AD proteomic signature identified in STAGE 1: DISCOVERY, a two-step validation of results was undertaken. In Step 6a: Primary Validation, western blotting of brain homogenates from 3xTg-AD mice and human samples (BLSA MFG) was performed and the subcellular localization of these proteins in the human brain was assessed using immunohistochemistry. In Step 6b: Secondary Validation, the signature was validated using orthogonal methods—i.e., mass spectrometry-based proteomics in: 1) AD and CN samples in the Mount Sinai brain bank; 2) 5xFAD transgenic mouse model of AD; and 3) single cell transcriptomics through RNA sequencing (scRNA-seq) from brain samples in the Religious Orders Study Memory and Aging Project (ROSMAP).

### Step 6a: Primary Validation

Western Blot in 3xTg-AD Mouse Model and Human Brain Samples: A subset of 6 proteins from the incipient AD proteomic signature was selected for western blot validation. These proteins were first assayed in a 3xTg-AD mouse model. This model expresses mutant forms of human-amyloid precursor protein (APP), presenilin-1 and tau, developing age-dependent accumulation of extracellular Aβ plaques, intracellular tau accumulation, oxidative stress and cognitive deficits (Oddo et al., Neuron 2003, 39:409-421). Proteins were chosen for validation because of prior evidence suggesting that they are targeted by approved and experimental drugs for other diseases (Table 4, Example 2) and may therefore present plausible novel drug repurposing opportunities in AD.

Age-matched (young and old) transgenic (n=6) and wild type (WT; n=6) male and female adult mice were used in all experiments. Mice were maintained on a standard NIH diet ad libitum in a 12-h light/dark cycle. All mice were housed in the National Institute on Aging (NIA), Baltimore. The animals were group housed where possible. All animal experiments were performed using protocols approved by the appropriate institutional animal care and use committee of the NIA.

Identical western blot analyses were performed in the MFG from human brain samples in the BLSA AD (n=11) and CN (n=11) individuals. These samples were a convenience subset of available BLSA samples used in STAGE 1: DISCOVERY. Demographic characteristics of the sample are described in Table 5 (Example 2).

For both mouse and human tissue, brain homogenates were solubilized in 1× RIPA buffer containing protease inhibitor cocktail (Roche). Standard western blot procedures were followed. Equal amounts of proteins were resolved on 4-20% gradient gels (BioRad) sodium dodecyl sulfate polyacrylamide gel electrophoresis (SDS-PAGE). Proteins were transferred to 0.2 μM pore size polyvinylidene difluoride (PVDF) membrane using the wet transfer system (BioRad). Membranes were blocked with 3% milk (BioRad) at room temperature for Ihr and then incubated with primary antibodies overnight at 4° C. The following primary antibodies were used: STAT3 (Cell Signaling Technology, 30835), LGALS8 (Novus, NBP2-75501), TOP1 (ThermoFisher Scientific, MA5-32228), DUSP3 (Abclonal, A12068), FYN1 (Invitrogen, MA1-19331) and YES1 (Proteintech, 20243-1-AP). Secondary horseradish peroxidase-conjugated antibodies and ECL prime (GE Healthcare Bio-Sciences) or SuperSignal West Femto Chemiluminescent Substrates (Thermo Fisher Scientific) were used to visualize signals on a ChemiDoc XRS+ system (BioRad Laboratories, Hercules, CA, USA). β-Actin was used for the loading control and normalization for total brain lysates. Digitized images were obtained, processed, and quantified with ImageLab version 6.1 (BioRad Laboratories).

Immunohistochemical Staining of Human Brain Tissue Samples: The autopsied brain samples were obtained at the Office of the Chief Medical Examiner (OCME) of the State of Maryland in Baltimore, and brains were accessioned as previously described (Sci Rep 2018; 8(1): 16895). Protocols authorized by the Institutional Review Board (IRB) of the State of Maryland Department of Health and Human Services and Johns Hopkins Medicine were followed. For the primary screen to determine whether proteins showed positive immunoreactivity (IR), at least two commercially available antibodies were used for each selected protein (see Table 7, Example 2). Proteins that screened IR positive, were then selected for secondary screening for subcellular localization.

All tissue sections were deparaffinized in xylene and rehydrated in 100% and 95% EtOH. To ascertain good preservation of tissues to be examined, sections of the cerebellum were screened with β-tubulin immunohistochemistry as previously described (Sci Rep 2018; 8(1): 16895). Antigen retrieval was performed using HistoVT (Nacalai) or Dewax and HIER buffer M (epredia) at 95 C for 20 min.

After washing with tap water, antigen retrieval was performed in 1 mM EDTA (pH8.0) (Invitrogen: 15575-038) by boiling for 4 min. Then, all samples were blocked in PBS (Phosphate Buffered Saline) with 5% normal goat serum (SIGMA), 5% normal donkey serum and 0.2% Triton x-100 for 1 hr at RT. Primary antibodies were incubated in blocking buffer for 16 h at 4° C. On the following day, samples were washed in PBS for 5 min×3, then Alexa fluor secondary antibodies in PBS with 0.5% Tween 20 were applied and incubated for 1 hr at RT. Samples were washed in PBS once for 5 min, then 5 μg/mL Hoechst 33258 in PBS were applied and incubated for 20 min at RT. Samples were washed in PBS once for 5 min. For quenching lipofuscin autofluorescence, TrueBlack® Lipofuscin Autofluorescence Quencher (Biotium: 23007) diluted 1/40 in 70% EtOH was applied to the samples and incubated for 50 seconds at RT. To facilitate the TrueBlack® reaction, samples were constantly swirled by hand during the incubation. Then, samples were washed in PBS for 5 min×3 and coverslipped using ProLong™ Gold Antifade reagent (Invitrogen: P36930). To enhance signal:noise ratio (SNR) of synaptic protein immunoreactivity, an ultrafast optical clearing method (FOCM) solution (Zhu et al., PNAS USA 2019, 116:11480-11489) mixed with a commercial antifade mounting solution was used. Stained tissue sections were kept at 4° C. at least 2 days before imaging. Immunofluorescent images were taken on a Zeiss LSM 700 confocal microscope in the Microscope Facility of the Johns Hopkins School of Medicine.

### Step 6b: Secondary Validation Subject Details and Data Acquisition

All secondary validation analyses on the publicly available datasets used were performed in collaboration with corresponding authors from the index publications and described below. Relevant analytic code and guidance on data use was requested directly from study authors.

Mount Sinai Brain Bank: Proteomic data from Brodmann area 36 brain region samples from the Mount Sinai brain bank sample comparing AD (n=39) and CN (n=23) individuals was obtained. These data were recently published and are available from the AMP-AD Knowledge Portal (https://adknowledgeportal.synapse.org) (Bai et al., Neuron 2020, 160:700). Proteins were quantified using acidic pH reverse phase LC-MS/MS. Demographic information for this cohort is available in the supplementary information from the source publication. AD diagnosis in this cohort was made using cognitive and neuropathological assessments as described previously (Wang et al., Sci Data 2018, 5:180185). Prior studies have indicated an appropriate level of complementarity between LC-MS/MS and SOMAscan methods, such that high degrees of concordance in proteomic quantification may be obtained across the two platforms. Differences in results across platforms have primarily been attributed to post-translational modification enrichment, suggesting that such modifications may better explain divergence of results across the two platforms than differences in protein abundance (Billing et al., J Proteomics 2017, 150:86-97).

5xFAD Mouse Model: Proteomic data were obtained from cortical brain samples from the 5xFAD transgenic mouse model of AD comparing transgenic (n=4) to wild type (WT; n=4) mice (Bai et al., Neuron 2020, 106:700). These data were published recently and are accessible at the Proteomics Identification Database (PRIDE; https://www.ebi.ac.uk/pride/). Protein quantification was achieved by acidic pH reverse phase LC-MS/MS. In this study, analyses were conducted at the 6-month time point which is associated with significant AD pathology and memory impairment in the 5xFAD mouse model (Oakley et al., J Neurosci 2006, 26:10129-10140).

ROSMAP scRNA-Seq: Study and Rush Memory and Aging Project (ROSMAP) (60) was downloaded from Synapse (https://www.synapse.org/#!Synapse:syn18485175). Code used to run the analyses in Mathys et al. (Nature 2019, 570:332-337) was requested from co-authors. Post-mortem data was collected from ROSMAP participants: 32 individuals (18 male and 14 female) in the AD category and 14 individuals (5 male and 9 female) in the CN category. The AD category included individuals with a clinical diagnosis of AD, as well as individuals with a clinical diagnosis of mild cognitive impairment (MCI) and no other condition contributing to cognitive impairment. The CN category included individuals with a clinical diagnosis of no cognitive impairment.

Tissue was profiled from the prefrontal cortex (Brodmann area 10) across eight major cell types in the aged dorsolateral prefrontal cortex: inhibitory neurons, excitatory neurons, astrocytes, oligodendrocytes, microglia, oligodendrocyte progenitor cells, endothelial cells, and pericytes. Additional details are provided in the index paper (Mathys et al., Nature 2019, 570:332-337). In the validation studies, analyses were restricted to inhibitory and excitatory neuron cell types in which a majority of transcripts for proteins in the incipient AD signature were quantified.

### Statistical Analyses

3xTg-AD Mouse Model: In the 3xTg-AD transgenic mouse model of AD, it was determined whether proteins were differentially expressed between young and old transgenic and WT mice. Protein expression was calculated as the ratio of protein staining intensity to its corresponding β-Actin intensity. Two-sample t-tests (parametric) were used to calculate differences in protein expression between 3xTg-AD transgenic and WT mice. Additionally, the Wilcoxon rank-sum test (non-parametric) was used to confirm that results were robust to distributional assumptions. Significant differences were indicated as p<0.05.

Mount Sinai Brain Bank: In the Mount Sinai Brain Bank sample, it was determined whether proteins were differentially abundant between AD and CN samples. 21/25 proteins from the incipient AD signature were quantified by mass spectrometry-based proteomics. Protein data were previously corrected for age and sex. A one-way test was performed for differential abundance between the AD and CN samples using the R package limma (Ritchie et al., Nucleic Acids Res 2015, 43:347). Significance was indicated as p<0.05.

5xFAD Mouse Model: In the 5xFAD transgenic mouse model of AD, it was determined whether proteins were differentially abundant between 5xFAD transgenic and WT mice. 21/25 proteins from the incipient AD signature were quantified in the cortex. Two-tailed Student's t-tests were performed. Protein levels were analyzed at the 6-month time point and significance was indicated as p<0.05.

ROSMAP scRNA-Seq: In the ROSMAP scRNA-Seq sample, it was determined whether mRNA levels of the genes associated with the proteins in the incipient AD signature were differentially abundant between AD and CN samples. 22/25 gene transcripts in both inhibitory and excitatory neurons from the proteins in the incipient AD signature were quantified. Each sample was scaled to have the same total read count (Robinson et al., Genome Biol 2010, 11:R25). To test differences between AD and CN, Wilcoxon rank sum tests were performed. Similar to the source publication (Mathys et al., Nature 2019, 570:332-337), each single-cell-specific sample from a participant was treated as an independent sample. Transcript levels for excitatory and inhibitory cell types between AD and CN were compared separately and significance was indicated as p<0.05.

### STAGE 3: Phenotypic Screening of Approved Drugs Targeting Selected Proteins in the Incipient AD Signature

Stage 3 evaluated whether approved/experimental drugs targeting proteins in the incipient AD signature could rescue distinct molecular phenotypes relevant to AD without adverse effects on cell viability. Three drugs known to target STAT3 were tested (Crizotinib, Napabucasin, and C188-9), as well as 1 drug targeting YES1 and FYN (Dasatinib). STAT3 and the Src family tyrosine kinases YES1 and FYN in these experiments were selected as they have been extensively studied as drug targets in cancer. The choice of candidate AD treatments in these experiments was based on the availability of FDA-approved drugs used in current clinical practice that target STAT3 (e.g. Crizotinib), YES1 and FYN (e.g. Dasatinib) or experimental drugs currently being tested in clinical trials (e.g. Napabucasin and C188-9). Drug concentrations tested are included in Table 8 (Example 3).

Lipopolysaccharide (LPS)-Induced Neuroinflammation: The murine microglial cell line BV-2 was cultivated in DMEM medium supplemented with 10% FCS, 1% penicillin/streptomycin and 2 mM L-glutamine (culture medium). For LPS stimulation assay, 5000 BV-2 cells per well (uncoated 96 well plates) were plated out and the medium was changed to treatment medium (DMEM, 5% FCS, 2 mM L-glutamine). After changing cells to treatment medium, drug compounds were administered 1 hour before LPS stimulation (Sigma-Aldrich; L6529; 1 mg/ml stock in ddH₂O, final concentration in well: 100 ng/ml (dilutions in medium)). Cells treated with vehicle, cells treated with LPS alone, as well as cells treated with LPS plus reference item (dexamethasone 10 μM, Sigma D4902) served as controls. After 24 h of stimulation, cell supernatants were collected for the cytokine measurement (V-PLEX Proinflammatory Panel 1 Mouse Kit, K15048D, Mesoscale) and cells were subjected to MTT assay.

AB Clearance: For the amyloid beta (Aβ) clearance assay, 20000 BV-2 cells per well (uncoated 96 well plates) were plated out. After changing cells to treatment medium, drug compounds were administered 1 hour before Aβ stimulation (Bachem 4061966; final concentration in well: 200 ng/ml (dilutions in medium)). Cells treated with vehicle and cells treated with Aβ alone served as controls. After 3 h of Aβ stimulation, cell supernatants were collected for the Aβ measurement and cells were carefully washed twice with PBS and thereafter lysed in 35 μL cell lysis buffer (50 mM Tris-HCl, pH 7.4, 150 mM NaCl, 5 mM EDTA, 1% SDS) supplemented with protease inhibitors. Supernatants and cell lysates were analyzed for human Aβ42 with MSD® V-PLEX Human Aβ42 Peptide (6E10) Kit (K151LBE, Mesoscale Discovery). The immune assay was carried out according to the manual and plates were read on the MESO QuickPlex SQ 120.

Tau Phosphorylation: SH-SY5Y-hTau441(V337M/R406W) cells were maintained in culture medium (DMEM medium, 10% FCS, 1% NEAA, 1% L-Glutamine, 100 g/mL Gentamycin, 300 μg/mL Geneticin G-418) and differentiated with 10 μM retinoic acid (RA) for 5 days changing medium every 2 to 3 days. Prior to the treatment, cells were seeded onto 24-well plates at a cell density of 2×10⁵ cells per well (DIV1). Drug compounds were applied on DIV2. After 24 h of incubation (DIV3), cells on 24-well plates were harvested in 60 μL RIPA-Buffer [50 mM Tris pH 7.4, 1% Nonidet P40, 0.25% Na-deoxy-cholate, 150 mM NaCl, 1 mM EDTA supplemented with freshly added 1 μM NaF, 0.2 mM Na-ortho-vanadate, 80 μM Glycerophosphate, protease (Calbiochem) and phosphatase (Sigma) inhibitor cocktail]. Protein concentration was determined by BCA assay (Pierce, ThermoFisher) and samples were adjusted to a uniform total protein concentration. Total Tau and phosphorylated Tau were determined by immunosorbent assay from Mesoscale Discovery (Phospho(Thr231)/Total Tau Kit K15121D, Mesoscale Discovery).

Trophic Factor Withdrawal: Primary cortical neurons from E18 C57Bl/6 mice were prepared as previously described. On the day of preparation (DIV1), cortical neurons were seeded on poly-D-lysine pre-coated 96-well plates at a density of 3×10⁴ cells per well. Every 4-6 days, a half medium exchange using full medium (Neurobasal, 2% B-27, 0.5 mM glutamine, 1% Penicillin-Streptomycin) was carried out. On DIV8, a full medium exchange to B-27 free medium (Neurobasal, 0.5 mM glutamine, 1% Penicillin-Streptomycin) was performed and drug compounds were applied thereafter. The experiment was carried out with n=6 technical replicates per condition, vehicle treated cells served as control. After 28 h on B-27 free medium, cells were subject to YOPRO/PI and MTT as well as LDH assay.

MTT Assay: MTT solution was added to each well in a final concentration of 0.5 mg/mL. After 2 h, the MTT containing medium was aspirated. Cells were lysed in 3% SDS and the formazan crystals were dissolved in isopropanol/HCl. Optical density was measured with a Cytation 5 (Biotek) multimode reader at wavelength 570 nm. Values were calculated as percent of control values (vehicle control or lesion control).

LDH Assay: Supernatants collected after treatment were subjected to the lactate dehydrogenase (LDH) toxicity assay by using the Cytotoxicity Detection Kit (Roche Diagnostics, Cat. No: 11 644 793 001). 70 μL of cell culture supernatant was transferred to clear 96-well plates. 70 μL freshly prepared reaction mixture was added to each well and the mixture was incubated for 1 h at room temperature protected from light. Absorbance was measured at 492 nm and 620 nm as reference wavelength with a Cytation 5 (Biotek) multimode reader. Values of culture medium were subtracted as background control. Values were calculated as percent of control values (vehicle control or lesion control).

YOPRO/PI Apoptosis and Necrosis Assay: YO-PRO™-1 (Invitrogen; Y3603) assay was carried out to detect apoptotic cells in combination with Propidium iodide (PI; P4864 Sigma Aldrich) staining for necrotic cells. Part of the supernatant of the cultivated cells was aspirated, so that 90 μL were remaining per well. 50 μM YO-PRO 1 solution was prepared out of the 1 mM YO-PRO 1 stock solution in DMSO. The stock solution was diluted in a ratio of 1:20 in PBS and Propidium iodide (PI) was added to the same stock to a final conc. of 1 μg/mL. 10 μL of this 50 μM YO-PRO 1/1μg/mL PI solution in PBS was added to the remaining 90 μL to result in a final concentration of 5 μM YO-PRO 1 in well. Incubation for 15 min in the incubator at 37° C. was performed (light protected). Supernatant was aspirated completely and discarded. 140 μL PBS was added to well. Plate was measured at the multimode-reader (Cytation 5, BioTek).

Aβ Secretion: H4-hAPP cells were cultivated in Opti-MEM supplemented with 10% FCS, 1% penicillin/streptomycin 200 μg/mL Hygromycin B and 2.5 μg/mL Blasticidin S. H4-hAPP cells were seeded into 96 well plates (2×10⁴ cells per well). On the next day, cells in 96 well plates were treated with compounds, R.I. (DAPT 400 nM) or vehicle. 24 h later, supernatants were collected for further Aβ measurements by MSD® (V-PLEX Aβ Peptide Panel 1 (6E10) Kit, K15200E, Mesoscale Discovery).

Statistical Analyses: Statistical analysis was performed in GraphPad Prism 9.1.2. Group differences were evaluated for each Test item separately by One-way ANOVA followed by Dunnett's multiple comparison test versus vehicle or lesion control.

### Example 1

### Overview and Stage 1—Alzheimer's Disease Proteomic Signature Discovery

### Overview

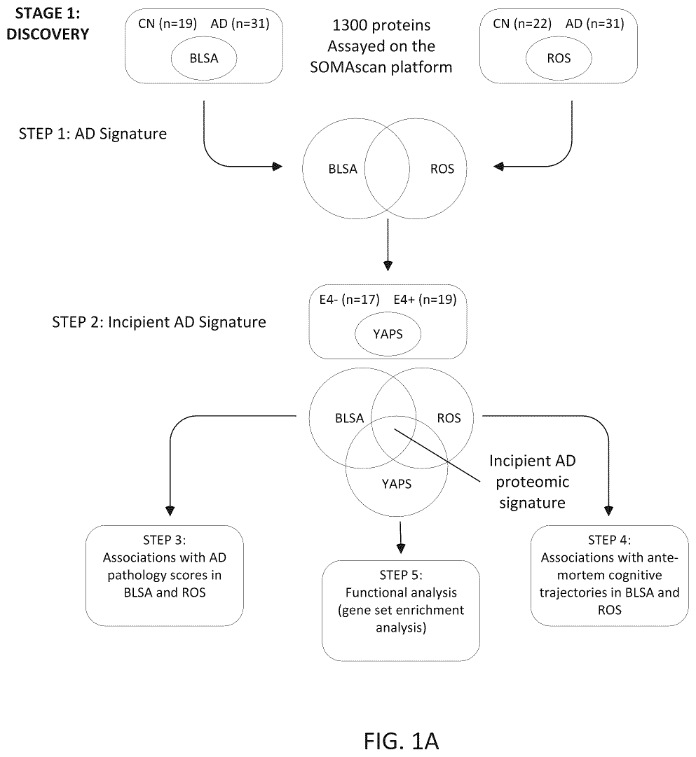

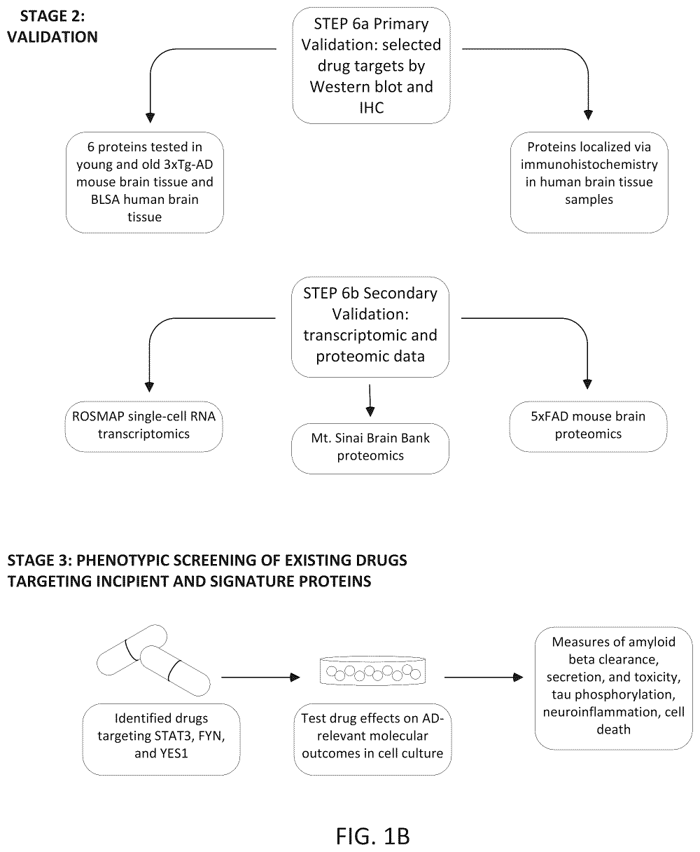

A three-stage study (FIGS. 1A, 1B) was undertaken to characterize the underlying biological pathways that connect the APOE genotype with the development of pathology that eventually leads to AD. First, proteins altered in the brains of AD individuals that are also dysregulated in young APOE 84 carriers and may therefore confer risk for future AD were identified. Brain samples from two independent cohorts (BLSA and ROS) of individuals with Alzheimer's disease (AD) were assayed on the SomaLogic aptamer-based proteomic platform, and proteins differentially abundant in both BLSA and ROS were defined as an AD proteomic signature. In Step 2, proteins in this AD proteomic signature were then assessed in an additional cohort (YAPS) of young individuals who were either APOE ε4 carriers or non-carriers. Proteins that differed in abundance across all 3 cohorts were defined as an incipient AD proteomic signature. In Step 3, associations between proteins in the incipient AD proteomic signature with severity of AD pathology (i.e., CERAD and Braak scores) were tested and in Step 4 associations with ante-mortem trajectories of cognitive performance prior to AD onset in the BLSA and ROS cohorts were tested. In Step 5, results of gene set enrichment analyses (GSEA) in the young sample (i.e., APOE ε4 carriers relative to non-carriers) were compared to the older adult samples (i.e., AD individuals relative to controls) to identify AD-related biologic pathways that may be altered in young APOE ε4 individuals.

Those proteins were then validated across multiple independent cohorts including brain tissue samples from human studies and two transgenic AD mouse models using orthogonal proteomic and transcriptomic methods. In the primary validation analysis (Step 6a: Primary Validation) a subset of proteins was selected from the incipient AD proteomic signature that are targets of both approved and experimental drugs for non-AD indications as the biological pathways represented by these proteins may present plausible novel therapeutic targets in AD. Their levels were assessed using western blotting (WB) in brain tissue samples from the 3xTg-AD mouse model of AD, as well as in AD and CN brain tissue samples from a subset of BLSA participants. We additionally assessed subcellular localization of some of these proteins using immunohistochemistry (IHC) in brain samples from participants without AD pathology. In Step 6b: Secondary Validation, validation analyses assessing all 25 proteins in the incipient AD proteomic signature were carried out in 3 publicly available datasets using orthogonal methods: mass-spectrometry-based human brain proteomics (Mt. Sinai Brain Bank), mass spectrometry-based mouse brain proteomics (5xFAD transgenic mouse model of AD), and a single-cell human neuronal RNA transcriptomic dataset (ROSMAP).

Finally, using phenotypic assays in cell culture models, drugs targeting three of these proteins, STAT3, YES1 and FYN, were demonstrated to rescue distinct molecular phenotypes relevant to AD pathogenesis, i.e., Aβ and tau pathology, neuroinflammation, and cell death.

### Stage 1

An AD proteomic signature was first identified by comparing brain tissue protein levels in two independent, older adult post-mortem samples of AD and age-matched cognitively normal controls (CN). Proteins identified in the AD proteomic signature were then evaluated in a young, post-mortem sample of APOE ε4 carriers (i.e., APOE ε3/4) and non-carriers to derive an incipient AD proteomic signature—i.e., the subset of differentially abundant proteins in both young APOE ε4 carriers relative to non-carriers and older adult AD individuals relative to CN (Step 2). To assess the relationship between the incipient AD proteomic signature and endophenotypes of AD, associations with severity of AD pathology at death (i.e., CERAD and Braak scores) (Step 3) and with ante-mortem trajectories of cognitive decline prior to the onset of AD (Step 4) were examined. AD-related biologic pathways that may be altered in young APOE ε4 individuals were identified by comparing results of gene set enrichment analyses (GSEA) in the young sample to the older adult samples (i.e., AD individuals relative to CN; Step 5).

### Demographic Characteristics

The characteristics of the three cohorts are summarized in Table 1. In the BLSA sample, the AD and CN groups did not differ significantly in age at death, sex, APOE ε4 carrier status, and postmortem interval (PMI). The AD group included a higher number of White participants (race) compared to CN samples. As expected, AD and CN groups varied significantly in MMSE score, severity of neuritic plaques (CERAD scores) and neurofibrillary tangles (Braak scores) with lower cognition and higher levels of pathology in the AD group.

In the ROS sample, the AD and CN groups did not vary significantly in race, APOE ε4 carrier status, and PMI. The AD group was significantly older at death and more likely female (sex) compared to CN. As expected, AD and CN groups varied significantly in MMSE score, severity of neuritic plaques (CERAD scores) and neurofibrillary tangles (Braak scores) with lower cognition and higher levels of pathology in the AD group.

Table 1 additionally summarizes differences across the BLSA and ROS cohorts. Considering the total sample, BLSA and ROS varied significantly in sex, race, and PMI. Comparing by group (e.g., BLSA AD/CN compared to ROS AD/CN respectively), the BLSA AD group had an earlier age of onset, a longer disease duration, was lower percentage female, higher MMSE score, higher Braak score, and a longer PMI compared to the ROS AD group.

The Young APOE Postmortem Study (YAPS) cohort had a mean age of 39 years, was approximately 50 percent APOE ε3/4 by design and contained 29 percent non-white individuals. There were no differences in other demographic characteristics between APOE ε4+ and APOE ε4− groups in YAPS.

TABLE 1
Cohort Demographics
Baltimore Longitudinal Study of Aging (BLSA)

| | Total Sample | | AD | | CN | |
|---|---|---|---|---|---|---|
| | N = 52 | | N = 31 | | N = 21 | |
| Age at death, | 86.26 | (10.50)† | 88.46 | (8.30)† | 83.02 | (12.63) |
| mean (SD) | | | | | | |
| Age of onset, | — | | 78.81 | (9.92)† | | |
| mean (SD) | | | | | | |
| Disease duration, | — | | 9.65 | (4.47)† | | |
| mean (SD) | | | | | | |
| Sex, n (% female) | 22 | (42.31)† | 15 | (48.39)† | 7 | (33.33) |
| Race, n (% white) | 49 | (94.23) | 31 | (100)* | 18 | (85.71)* |
| APOE e4 carrier, | 14 | (28.00) | 9 | (31.03) | 5 | (23.81) |
| n (%) | | | | | | |
| MMSE, mean (SD) | 24.66 | (5.59) | 21.60 | (6.05)*† | 28.06 | (1.98)* |
| CERAD, mean (SD) | 1.73 | (1.34) | 2.77 | (0.43)* | 0.19 | (0.40)* |
| Braak, mean (SD) | 4.10 | (1.67) | 5.13 | (1.12)*† | 2.57 | (1.08)* |
| Postmortem interval | 15.58 | (10.01)† | 16.25 | (11.73)† | 14.53 | (6.63)† |
| (hours), mean (SD) | | | | | | |

Religious Orders Study (ROS)

| | Total Sample | | AD | | CN | |
|---|---|---|---|---|---|---|
| | N = 53 | | N = 31 | | N = 22 | |
| Age at death, | 90.58 | (6.53)† | 92.81 | (5.45)*† | 87.44 | (6.75)* |
| mean (SD) | | | | | | |
| Age of onset, | — | | 89.14 | (5.71)† | — | |
| mean (SD) | | | | | | |
| Disease duration, | — | | 3.67 | (2.94)† | — | |
| mean (SD) | | | | | | |
| Sex, n (% female) | 40 | (75.47)† | 27 | (87.10)*† | 13 | (59.09)* |
| Race, n (% white) | 53 | (100.00) | 31 | (100.00) | 22 | (100.00) |
| APOE e4 carrier, | 13 | (25.49) | 9 | (31.03) | 4 | (18.18) |
| n (%) | | | | | | |
| MMSE, mean (SD) | 19.95 | (10.10) | 12.77 | (8.43)*† | 28.50 | (1.59)* |
| CERAD, mean (SD) | 1.64 | (1.26) | 2.58 | (0.50)* | 0.32 | (0.65)* |
| Braak, mean (SD) | 3.89 | (1.33) | 4.65 | (0.66)*† | 2.82 | (1.30)* |
| Postmortem interval | 9.05 | (5.32)† | 8.67 | (5.36)† | 9.59 | (5.35)† |
| (hours), mean (SD) | | | | | | |

Young APOE postmortem study (YAPS)

| | Total Sample | | APOE ε4+ | | APOE ε4− | |
|---|---|---|---|---|---|---|
| | N = 35 | | N = 18 | | N = 17 | |
| Age at death, | 38.97 | (5.95) | 39.06 | (6.15) | 38.88 | (5.93) |
| mean (SD) | | | | | | |
| Sex, n (% female) | 13 | (37.14) | 6 | (33.33) | 7 | (41.18) |
| Race, n (% white) | 25 | (71.43) | 14 | (77.78) | 11 | (64.71) |
| Postmortem interval | 23.14 | (11.42) | 24.39 | (12.46) | 21.82 | (10.42) |
| (hours), mean (SD) | | | | | | |

AD: Alzheimer's disease; CN: Cognitively normal; ASY: Asymptomatic AD; Disease duration: age death - age onset; MMSE: mini mental state exam (last available prior to death); APOE ε4+: APOE e4 carrier; APOE ε4−: APOE e4 non carrier; note: the Wilcoxon rank-sum (Mann-Whitney) test and chi-squared test was used to test for differences in continuous and binary variables respectively.
*p < 0.05 comparing AD to CN
†p < 0.05 comparing BLSA to ROS (e.g. AD in BLSA compared to AD in ROS)

### AD Proteomic Signature

To determine a brain proteomic signature of AD, protein levels were compared between AD and CN individuals in the ROS and BLSA cohorts in the inferior temporal gyrus (ITG) and middle frontal gyrus (MFG) brain regions. The results are shown in FIGS. 2A-2D. FIG. 2A shows results of separate proportional odds models to identify an incipient AD proteomic signature; by comparing AD and CN individuals in BLSA and ROS cohorts, an AD proteomic signature (i.e., 120 unique proteins across the ITG and MFG) was identified; Type I error was controlled using an FDR p-value threshold <0.10 and the statistical significance of the 2-way intersection was tested using the SuperExactTest (p<0.00001). These 120 proteins were then analyzed in the YAPS cohort and to establish an incipient AD proteomic signature (25 proteins indicated in red) as the overlap across all 3 cohorts. The statistical significance of this 3-way intersection was tested using the SuperExactTest (p<0.00001). This intersection represents proteins differentially abundant in young APOE ε4 carriers as well as in AD individuals from two independent cohorts—BLSA and ROS. FIG. 2B shows volcano plots highlighting proteins in BLSA and ROS used to define the 120 protein AD proteomic signature: proteins above the FDR threshold and significant in both BLSA and ROS, where the dashed line represents an FDR threshold of p=0.10 and the vertical line represents an OR of 1 (log 10(OR)=0) indicating no difference in protein concentration between AD and CN. 1300 proteins are included in the background; gray points indicate proteins that do not meet the FDR threshold in both BLSA and ROS (i.e., not overlapping across cohorts); blue and red points indicate proteins significantly lower and higher respectively in AD individuals in both BLSA and ROS. Because all significant proteins in BLSA were from the MFG and the majority of significant proteins in ROS were from the ITG, only these regions were visualized. FIG. 2C shows volcano plots highlighting proteins from the 120 protein AD proteomic signature that overlap with proteins differentially abundant in YAPS ITG or MFG (i.e., the 25-protein incipient AD signature) where the dashed line represents a p-value threshold of 0.05, and the black vertical line represents an OR of 1 (log10(OR)=0) indicating no difference in protein concentration between APOE ε4+ and APOE ε4− groups. The 120 proteins comprising the AD proteomic signature are included in the background; gray points indicate proteins that do not meet the p-value threshold (i.e., not overlapping across all 3 cohorts); blue and red points indicate proteins significantly lower and higher in abundance respectively in APOE ε4+ individuals. FIG. 2D shows differences in protein levels in each cohort for the 25 proteins included in the incipient AD proteomic signature; for the proteins significant in both the ITG and MFG, the more significant brain region (i.e., smaller p-value in the proportional odds model) was visualized. The y-axis indicates the log10(odds ratio), with a positive value indicating a higher concentration of the protein in APOE ε4+ compared to APOE ε4− individuals (YAPS cohort) or AD compared to CN individuals (BLSA and ROS cohorts) and a negative value indicating a lower concentration of the protein in group comparisons. The x-axis indicates the protein name, and bars are shaded according to cohort.

Of the 1300 proteins measured, in the BLSA 0 and 254 differentially abundant proteins (false discovery rate (FDR)-adjusted p-value<0.10) were identified in the ITG and MFG, respectively. In the ROS cohort, 244 and 4 differentially abundant proteins (FDR-adjusted p-value<0.10) were identified in the ITG and MFG, respectively (244 unique proteins in total).

To establish an AD proteomic signature across both cohorts, the intersection between differentially abundant proteins in the BLSA and ROS cohorts was evaluated. This resulted in 120 unique proteins that were differentially abundant in both BLSA and ROS in either the ITG or MFG (FIG. 2A). Of these 120 proteins, 84 were lower in AD compared to CN and 34 were higher in AD. Only 2 displayed inconsistent direction between ROS and BLSA (i.e., higher in one cohort and lower in the other).

### Incipient AD Proteomic Signature

A primary motivation was to determine proteomic alterations in young APOE ε4 carriers that may drive risk for subsequent AD. A determination was made regarding which of the 120 proteins altered in both AD cohorts were also dysregulated between young APOE ε4 carriers and non-carriers in the YAPS cohort. Of the 120 proteins, 16 and 14 proteins were significantly different (p<0.05) between APOE ε4 carriers and non-carriers in the ITG and MFG, respectively, resulting in 25 unique proteins defining the incipient AD proteomic signature (FIG. 2A). Of these proteins, 24 were increased in young APOE ε4 carriers, while 1 protein was reduced relative to APOE ε4 non-carriers (FIG. 2B). For proteins significantly altered in both ITG and MFG brain regions, direction was always consistent in the YAPS cohort. Nearly all proteins in the incipient AD proteomic signature displayed an opposite direction of association in YAPS compared to BLSA and ROS, such that protein levels were increased in young APOE ε4 carriers relative to non-carriers and reduced in AD relative to controls. Of the 25 proteins, only one protein (TBP) displayed an inconsistent direction of association between BLSA and ROS (increased in YAPS and ROS but decreased in BLSA; indicated by an asterisk in FIG. 2D).

The statistical significance of the overlap between these three proteomic signatures was tested by implementing multi-set intersection analysis through the SuperExactTest developed by Wang et al. (Sci Rep 2015, 5:16923). This procedure computes the statistical distributions of multi-set intersections using combinatorial theory and efficiently calculates their exact probabilities. The YAPS, BLSA, and ROS proteomic signatures, including 86, 254, and 244 significantly altered proteins, respectively, share 25 proteins, and this intersection, defined as the incipient AD proteomic signature, is highly significant (SuperExactTest p<0.00001). This analysis indicates that the observed signature of 25 proteins in the incipient AD proteomic signature significantly exceeds the expected, or null, signature of 3.15 proteins (i.e., 7.9-fold enrichment) had the 3 cohort-specific signatures been selected at random from the original list of 1300 proteins. Additionally, the overlap between ROS and BLSA proteomic signatures, defined as the AD proteomic signature, consisting of 120 significant proteins, is similarly highly significant (SuperExactTest p<0.00001) and exceeds the expected overlap of 47.6 proteins (i.e., 2.52-fold enrichment) (Table 2).

TABLE 2
SuperExactTest Intersection Analysis of Protein Signatures

| Intersecting Protein | Expected | Actual | Fold | |
|---|---|---|---|---|
| Signatures | Overlap | Overlap | Enrichment | p-value |
| BLSA + ROS (i.e., AD | 47.6 | 120 | 2.52 | <0.00001 |
| proteomic signature) | | | | |
| BLSA + ROS + YAPS | 3.15 | 25 | 7.9 | <0.00001 |
| (i.e., incipient AD | | | | |
| proteomic signature) | | | | |

To determine if proteins in the incipient AD proteomic signature were functionally related, pathway enrichment analysis was performed using the 25-protein list (Table 3). This analysis identified several significantly enriched pathways among the signature, including cytokine signaling, tyrosine kinase signaling, cell migration, and other signaling pathways. The protein-protein interactions in the signature were examined using the StringDB database (https://string-db.org). This identified numerous significant interactions, including identification of STAT3, FYN, and YES1 as central nodes.

TABLE 3
Incipient AD Proteomic Signature

| Protein | Protein | Protein |
|---|---|---|
| AKT2 | IFNL2 | PRKCI |
| CAMK2B | KPNB1 | RNASEH1 |
| CAMK2D | LGALS8 | SNX4 |
| CCL19 | LRPAP1 | STAT3 |
| DAPK2 | LRRTM3 | TBP |
| DUSP3 | MAPK12 | TOP1 |
| FYN | METAP1 | YES1 |
| GRP | PDPK1 | |
| HMOX2 | PRKCB | |

### Sensitivity Analysis

To verify that the differential abundance of proteins in the incipient AD proteomic signature was not driven by the presence of APOE ε4+ AD individuals, APOE ε4+ individuals from the BLSA and ROS cohorts were excluded. Results were generally consistent with the main analysis: 24 out of 25 proteins from the incipient AD proteomic signature remained statistically significant. Similarly, the sample was restricted to only APOE ε4+ individuals in ROS and BLSA to perform similar comparisons between AD and CN. Despite the small sample size in this comparison (AD/CN N=9/4 in BLSA and 9/4 in ROS), 7 proteins from the incipient AD proteomic signature remained statistically significant, and the direction of association for all 25 proteins remained consistent with the main analysis.

The incipient AD proteomic signature is associated with severity of Alzheimer's disease pathology: Braak and CERAD scores.

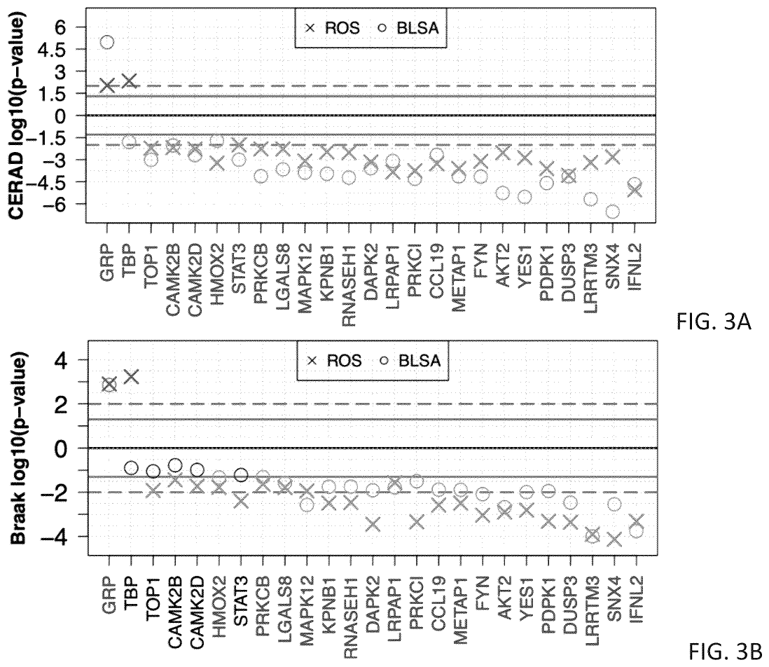

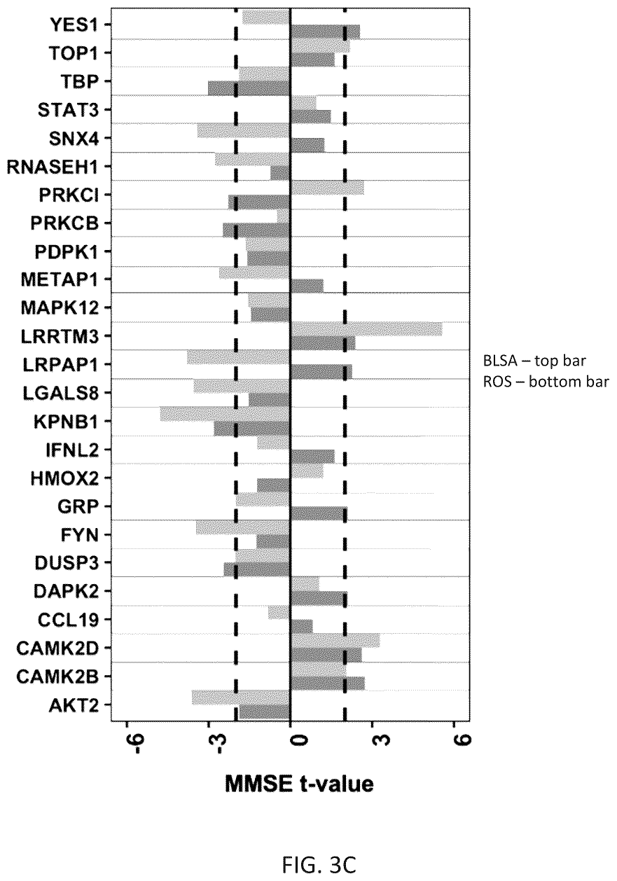

FIGS. 3A-3B show associations between the incipient AD proteomic signature with severity of AD pathology and longitudinal trajectories of ante-mortem cognitive performance. Results of partial correlation analyses between levels of the 25 proteins comprising the incipient AD proteomic signature and (FIG. 3A) Braak and (FIG. 3B) CERAD scores in the BLSA and ROS cohorts in either the ITG or MFG (brain region with lower p-value visualized). The y-axis indicates the log10(p-value), and protein names are indicated on the x-axis. Positive values indicate that a higher protein concentration was associated with a higher pathology score, while negative values indicate that a higher protein concentration was associated with a lower pathology score. Values beyond the solid pink line indicate a p-value<0.05; values beyond the dashed pink line indicate a p-value<0.01. Positive significant values (red) and negative significant values (green) indicate that higher protein concentration is associated with higher or lower neurofibrillary tangle pathology (Braak scores)/neuritic plaque burden (CERAD scores), respectively. Non-significant associations are indicated in black. Protein names in blue indicate proteins for which a significant association was found in both ROS and BLSA. BLSA: Baltimore Longitudinal Study of Aging; ROS: Religious Orders Study; CERAD: Consortium to Establish a Registry for Alzheimer's Disease; AD: Alzheimer's disease; ITG: inferior temporal gyrus; MFG: middle frontal gyrus; MMSE: Mini Mental State Examination.

In the ITG and MFG, 11 of 25 and 15 of 25 proteins respectively from the incipient AD proteomic signature were also significantly (p<0.05) associated with severity of neurofibrillary pathology (Braak scores) with consistent direction in both the BLSA and ROS cohorts (FIG. 3A). In the ITG and MFG, 11 of 25 and 21 of 25 proteins respectively from the incipient AD proteomic signature were significantly (p<0.05) associated with severity of neuritic plaque burden (CERAD scores) with consistent direction in both the ROS and BLSA cohorts (FIG. 3B).

With the exception of gastrin-releasing peptide (GRP), which was associated with higher CERAD scores in the MFG of both cohorts, all significant shared associations in the BLSA and ROS cohorts indicated that a lower protein level was associated with a higher pathology score. In both BLSA and ROS, 7 proteins (CCL19, METAP1, DUSP3, SNX4, IFNL2, YES1, PDPK1) displayed significant associations with both CERAD and Braak scores in both the ITG and MFG brain regions.

The incipient AD proteomic signature is associated with ante-mortem trajectories of cognitive performance prior to development of AD.

FIG. 3C shows associations between proteins comprising the incipient AD proteomic signature and longitudinal trajectories in Mini-Mental State Examination (MMSE) scores in AD individuals in the BLSA and ROS in the ITG or MFG (more significant, i.e. lower p-value, brain region visualized). A negative t-value indicates a negative association between the protein level and the slope in MMSE scores over time (i.e., a higher protein level is associated with a faster/increased decline in MMSE). A positive t-value indicates a positive association between the protein level and the slope in MMSE scores over time (i.e., a higher protein level is associated with a slower/reduced decline in MMSE). Values beyond the dashed lines indicate a p-value<0.05. BLSA:

In either the ITG or MFG, 20 of 25 proteins in the incipient AD proteomic signature were significantly (p<0.05) associated with longitudinal trajectories of Mini Mental State Examination (MMSE) scores in AD individuals from BLSA or ROS (FIG. 3C). Six proteins were significantly associated with longitudinal trajectories of MMSE scores with a consistent direction in both BLSA and ROS: CAMK2B, CAMK2D, and LRRTM3 were associated with a slower decline in MMSE, while DUSP3, KPNB1, and LRPAP1 were associated with a faster decline in MMSE in both cohorts.

GSEA identifies pathways dysregulated in both young APOE ε4 carriers and AD.

In order to derive a global understanding of molecular pathways and biological functions implicated by the proteomic alterations associated with APOE ε4 carrier status in young individuals, gene set enrichment analysis (GSEA) was performed on all proteins included in our dataset. All 1300 measured proteins were utilized in YAPS, BLSA, and ROS so that analyses were not biased towards only highly significant proteins but rather provided a comprehensive overview of proteomic alterations in each cohort. In the YAPS cohort, this analysis identified 44 significantly enriched gene sets (FDR adjusted p-value<0.05) in either the ITG or MFG. The majority (41 of 44) of these gene sets were overexpressed (i.e., positive normalized enrichment score; NES) in the YAPS cohort, indicating an upregulation of these pathways and their biological functions in young APOE ε4 carriers relative to non-carriers. These results signal an alteration in several biological functions including calcium signaling, synaptic transmission, insulin signaling, immune response, and cell-cell adhesion.

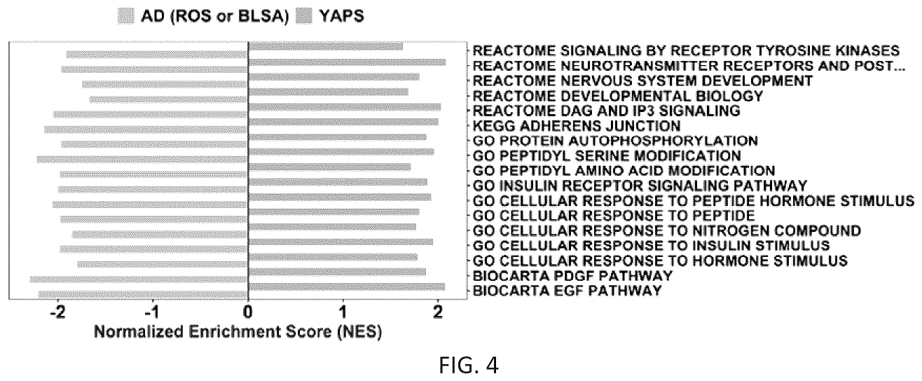

An analysis was performed to identify whether any of the gene sets enriched in the YAPS cohort were similarly enriched in either of the AD cohorts, i.e. BLSA or ROS. GSEA was performed with the same gene sets analyzed in the YAPS cohort for BLSA and ROS separately. FIG. 4 shows results of the GSEA analyses for the 17 gene sets identified as dysregulated in both YAPS and at least one AD cohort. Comparing overlapping gene sets between YAPS and AD cohorts (ROS/BLSA), gene sets had significant and opposite expression in YAPS relative to the AD cohorts. The gene set name is given on the y-axis, and NES is provided on the x-axis. A positive NES indicates a significant positive enrichment of the gene set, such that this pathway may be overexpressed in the cohort. A negative NES indicates a negative enrichment of the gene set, such that this pathway may be under-expressed in the cohort. Bars on the left indicate the AD cohort (ROS or BLSA), and bars on the left indicate the YAPS cohort. BLSA: Baltimore Longitudinal Study of Aging; ROS: Religious Orders Study; YAPS: Young APOE Postmortem Study; FDR: false discovery rate; GSEA: gene set enrichment analysis; NES: normalized enrichment score.

This analysis showed that 17 of the 44 significantly enriched gene sets in YAPS were also enriched in either BLSA or ROS individuals (FDR adjusted p-value<0.05). Furthermore, the significance of this overlap was tested by performing Fisher's exact ratio tests between the number of overlaps at different FDR thresholds (0.05, 0.1 and 0.25), which gave Fisher test p-values<4.8×10⁻¹² indicating a statistically significant overlap in enriched gene sets between the YAPS and BLSA/ROS cohorts independent of the chosen FDR threshold.

### Example 2

### Stage 2—Validation

In step 6a, a subset of six proteins from the incipient AD proteomic signature that are targets of both approved and experimental drugs for non-AD indications was selected. The rationale was that the biological pathways represented by these proteins may reflect plausible novel therapeutic targets in AD. We assessed their levels using western blotting in brain tissue samples from the 3xTg-AD mouse model of AD as well as AD and CN human brain tissue samples. We additionally assessed the subcellular localization of these proteins in the human brain using immunohistochemistry. (Step 6b: Secondary validation) - we additionally assessed protein and transcript levels of all 25 proteins comprising the incipient AD proteomic signature in multiple publicly available datasets using orthogonal methods, including mass spectrometry-based proteomics in an independent cohort of AD and CN human brain tissue samples, the 5xFAD transgenic mouse model of AD and single cell neuronal transcriptomic data from AD and CN individuals.

### Primary Validation: Western Blot and Immunohistochemical Localization

### Western Blot Analyses in 3xTg-AD Brain Homogenates

A subset of 6 proteins out of the 25 proteins in the incipient AD proteomic signature was first validated using western blotting in brain homogenates from the 3xTg-AD transgenic mouse model of AD. This model expresses mutant forms of human-amyloid precursor protein (APP), presenilin-1 and tau, developing age-dependent accumulation of extracellular Aβ plaques, intracellular tau accumulation, oxidative stress and cognitive deficits (Oddo et al., Neuron 2003, 39:409-421). These proteins were selected based on prior evidence suggesting that they are targeted by approved and experimental drugs for other diseases (Table 4) and may therefore represent plausible therapeutic targets in AD through repurposing of existing drugs.

TABLE 4
Candidate Proteins for Primary Validation Studies in 3×Tg AD Mice

| | | | Antibody |
|---|---|---|---|
| | Approved/experimental | | used in |
| | drugs targeting | | primary |
| | protein/gene | | validation |
| Protein | of interest | Disease indication | studies |
| STAT3 | Crizotinib, Napabucasin, | Lung cancer | Cell Signaling |
| | C188-9 (1) | | Technology, |
| | | | 30835 |
| DUSP3 | MLS-0437605 (2) is a | Arterial thrombosis | Abclonal, |
| | specific inhibitor of | | A12068 |
| | dual-specificity | | |
| | phosphatase 3 | | |
| | (DUSP3) | | |
| LGALS8 | Methyl-β-D- | lung, kidney, | Novus, |
| | galactomalonyl | prostate, and breast | NBP2-75501 |
| | phenyl esters are potent | cancer, osteoporosis, | |
| | Galectin-8 antagonists | rheumatoid arthritis | |
| | (3) | | |
| YES1 | Dasanitib (4) | Breast, head and | Proteintech, |
| | | neck, and colorectal | 20243-1-AP |
| | | cancers | |
| TOP1 | Irinotecan (5) | Lung cancer | ThermoFisher |
| | | | Scientific, |
| | | | MA5-32228 |
| FYN1 | Dasatinib (4) | Breast, head and | Invitrogen, |
| | | neck, and colorectal | MA1-19331 |
| | | cancers | |

1. You et al., Oncotarget 2015, 6: 40268-40282.
2. Monteiro et al., Rev Physiol Biochem Pharmacol 2019, 176: 1-35.
3. Patel et al., J Med Chem 2020, 63: 11573-11584.
4. Monero et al., Clin Cancer Res 2011, 17: 5546-5552.
5. Leonard et al., Anticancer Drugs 2017, 28: 1086-1096.

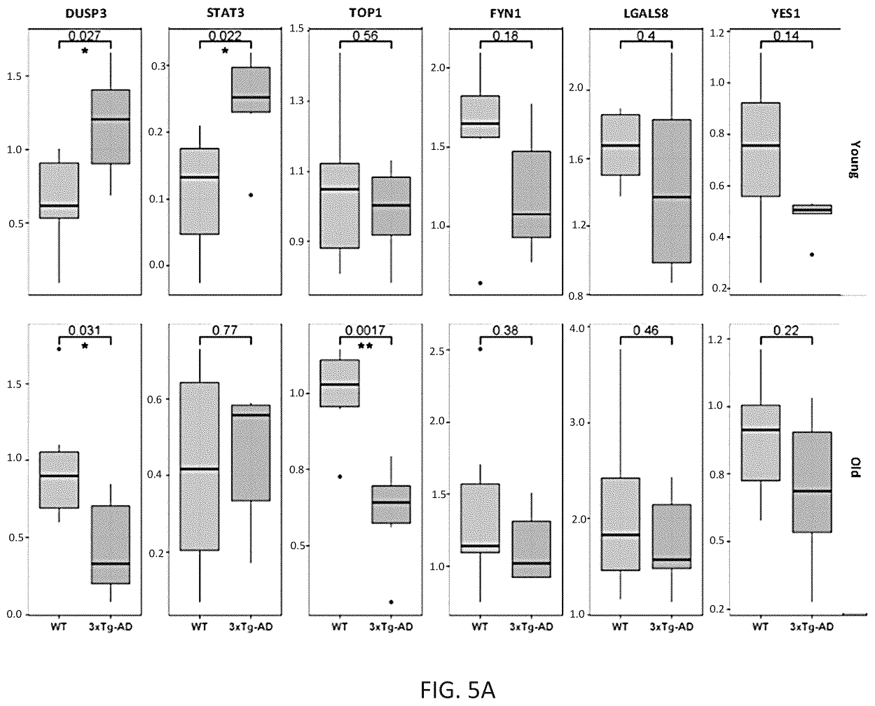

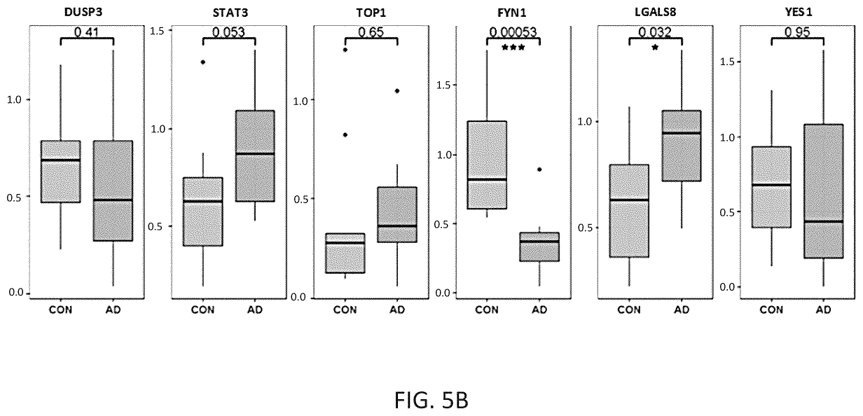

FIG. 5A shows results of primary validation analyses performed using western blotting in 3xTg-AD transgenic mice (AD) compared to wild type (WT). The 3xTg-AD (n=6) and WT (n=6) mice included young (38-40 weeks) and old (83-136 weeks) mice. Box plots represent differences in protein levels in brain homogenates between 3xTg-AD (blue) and WT (red) mice). The top and bottom row indicate protein levels in young and old mice respectively. Protein levels that were significantly different between 3xTg-AD and WT or AD and CN are indicated with * (p<0.05) or ** (p<0.01). 3xTg-AD: 3xTg-AD transgenic mouse model of AD; AD: Alzheimer's disease; WT: wild type; CN: cognitively normal; DUSP3: dual-specificity phosphate 3; STAT3: signal transducer and activator of transcription 3; TOP1: DNA topoisomerase I; FYN1: FYN proto-oncogene tyrosine kinase; LGALS8: Galectin8; YES1: YES proto-oncogene tyrosine kinase.

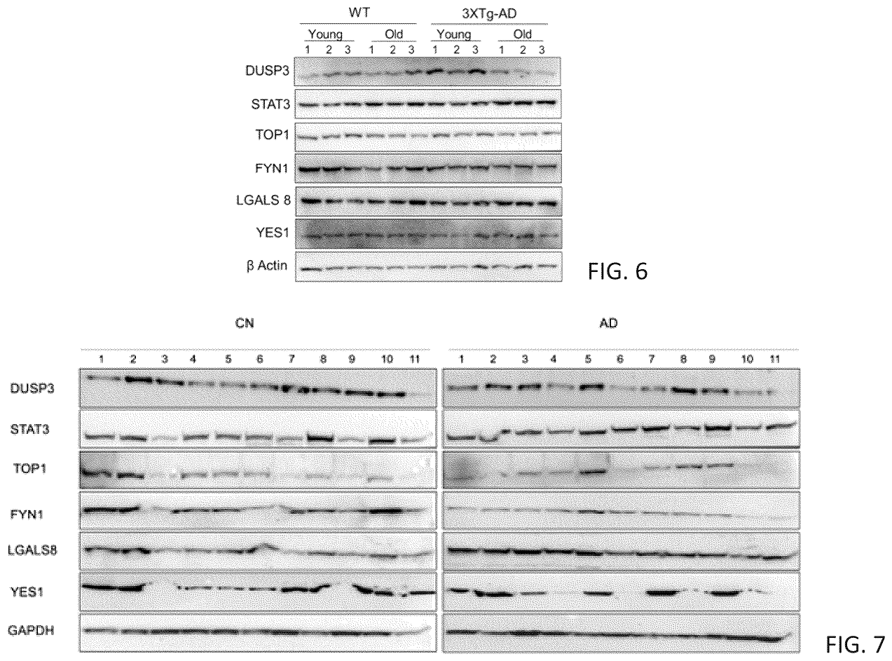

Of the 6 proteins chosen, 3 showed significantly altered levels in the brains of transgenic 3xTg-AD mice relative to wild type (WT; p<0.05). In young transgenic mice aged 38-40 weeks, protein levels of DUSP3 and STAT3 were higher in transgenic mice relative to WT, whereas in older mice aged 83-136 weeks, protein levels of DUSP3 and TOP1 were lower in transgenic mice relative to WT. Western blot images are included in FIG. 6.

### Western Blot Analyses in BLSA Human Brain Homogenates

The same set of 6 proteins from the incipient AD proteomic signature was next validated using western blotting in brain homogenates from a subset of AD and CN MFG brain tissue samples from the BLSA. The subject characteristics are shown in Table 5 and the results are shown in FIG. 5B. Box plots represent differences in protein levels in brain homogenates between AD (blue; n=11) and CN (red; n=11). Protein levels that were significantly different between 3xTg-AD and WT or AD and CN are indicated with * (p<0.05) or ** (p<0.01). 3xTg-AD: 3xTg-AD transgenic mouse model of AD; AD: Alzheimer's disease; WT: wild type; CN: cognitively normal; DUSP3: dual-specificity phosphate 3; STAT3: signal transducer and activator of transcription 3; TOP1: DNA topoisomerase I; FYN1: FYN proto-oncogene tyrosine kinase; LGALS8: Galectin8; YES1: YES proto-oncogene tyrosine kinase.

TABLE 5
Demographic Characteristics

| | Total Sample | | AD | | CN | |
|---|---|---|---|---|---|---|
| | N = 22 | | N = 11 | | N = 11 | |
| Age at death, mean (SD) | 84.50 | (11.06) | 86.59 | (10.63) | 82.41 | (11.58) |
| Sex, n (% female) | 7 | (31.82) | 6 | (54.55)* | 1 | (9.09)* |
| Race, n (% white) | 21 | (95.45) | 11 | (100) | 10 | (90.91) |
| APOE e4 carrier, n (%) | 5 | (22.73) | 3 | (27.27) | 2 | (18.18) |
| MMSE, mean (SD) | 26.4 | (5.04) | 22.00 | (6.96)* | 28.60 | (1.35)* |
| CERAD, mean (SD) | 1.50 | (1.47) | 2.91 | (0.30)* | 0.09 | (0.30)* |
| Braak, mean (SD) | 4.09 | (1.74) | 5.55 | (0.82)* | 2.64 | (1.03)* |
| Post mortem interval (hours), | 15.35 | (6.14) | 13.73 | (5.52) | 17.33 | (6.60) |
| mean (SD) | | | | | | |

Wilcoxon rank-sum (Mann-Whitney) test and chi-squared test used to test for differences in continuous and binary variables, respectively.
*p < 0.05.

Of the 6 proteins, 2 showed significantly altered levels in AD brain tissue compared to CN (p<0.05; FIG. 5B). FYN levels were reduced in AD, while LGALS8 levels were higher in AD relative to controls. STAT3 levels (marginally significant) were higher in AD relative to CN (p=0.053) (FIG. 5B). Western blot images are included in FIG. 7.

### Subcellular Localization of Proteins from the Incipient AD Signature in Human Brain

These analyses attempted to establish the subcellular localization of selected proteins from the incipient AD signature in the human brain. the goal in these experiments was to derive additional insights into plausible physiological roles of these proteins. An examination of whether the subset of 6 proteins assayed by western blotting in Step 6a (FIGS. 5A-5B, i.e., Primary Validation) could be detected in cortex within the non-diseased human brain using immunohistochemical colocalization of the proteins within distinct subcellular compartments was performed. Formalin-fixed paraffin embedded (FFPE) tissue samples from the inferior parietal cortex obtained from five autopsy brain samples were used. The autopsies were performed at the Office of the Chief Medical Examiner (OCME) of the State of Maryland in Baltimore and brains were obtained as previously described (Kageyama et al., Sci Rep 2018, 8;16895). Sample demographics are included in Table 6. The brains were confirmed free of AD pathology determined by standard neuropathologic criteria according to CERAD guidelines (Mirra et al., Neurology 1991, 41:479-486). Several commercially available antibodies were screened for positive immunoreactivity (at least 2 antibodies for each selected target protein; Table 7).

TABLE 6

| Sample # | Sex | Age | Race |
|---|---|---|---|
| 1 | F | 45 | White |
| 2 | F | 49 | White |
| 3 | M | 51 | White |
| 4 | M | 51 | White |
| 5 | F | 55 | Black |

TABLE 7

| Antibody | Company | Catalog # | Host | Dilution | Target |
|---|---|---|---|---|---|
| Stat3 (D1B2J) | CST | 30835 | Rabbit | 1/100 | Stat3 |
| Stat3 (F-2) | Santa Cruz | sc-8019 | Mouse | 1/50 | Stat3 |
| STAT3 | R&D | mab1799 | Mouse | 1/100 | Stat3 |
| Galectin-8 | Novus | NBP2- | Rabbit | 1/100 | Galectin 8 (LGALS8) |
| (JB85-35) | | 75501 | | | |
| Galectin-8 | R&D | AF1305 | Goat | 1/100 | LGALS8 |
| Galectin-8 | Sigma- | G5671 | Mouse | 1/100 | LGALS8 |
| | Aldrich | | (IgM) | | |
| Fyn | abcam | ab184276 | Rabbit | 1/250 | FYN Proto-Oncogene, Src |
| (EPR19636) | | | | | Family Tyrosine Kinase |
| Fyn (FYN-01) | Novus | NB500-517 | Mouse | 1/100 | FYN Proto-Oncogene, Src |
| | | | | | Family Tyrosine Kinase |
| Yes1 | proteintech | 20243-AP | Rabbit | 1/100 | YES Proto-Oncogene 1, Src |
| | | | | | Family Tyrosine Kinase |
| Yes | R&D | MAB32051 | Mouse | 1/100 | YES Proto-Oncogene 1, Src |
| | | | | | Family Tyrosine Kinase |
| DUSP3 | Abclonal | A12068 | Rabbit | 1/100 | Dual Specificity Phosphatase |
| | | | | | 3 |
| VHL (DUSP3) | R&D | mab1654 | Mouse | 1/50 | Dual Specificity Phosphatase |
| | | | | | 3 |
| TOP1 | Invitrogen | MA5-32228 | Rabbit | 1/50 | DNA Topoisomerase I |
| TOP1 | Invitrogen | MA1-25506 | Mouse | 1/100 | DNA Topoisomerase I |
| MAP2 | abcam | ab92434 | Chicken | 1/1000 | Neuron |
| | | | (IgY) | | |
| MTCO1 | abcam | ab14705 | Mouse | 1/100 | Mitochondria |
| Rab5 | abcam | ab109534 | Rabbit | 1/200 | Early endosome |
| LAMP1 | abcam | ab25630 | Mouse | 1/10 | Lysosome |
| Synaptophysin | abcam | ab8049 | Mouse | 1/50 | Presynapse |
| (SY38) | | | | | |
| PSD95 | abcam | ab13552 | Mouse | 1/100 | Excitatory postsynapse |

Two target proteins (i.e., DUSP3 and LGALS 8) showed good signal to noise ratio (SNR) with their corresponding antibodies and were selected for immunohistochemical co-localization experiments using additional antibodies against specific subcellular compartments.

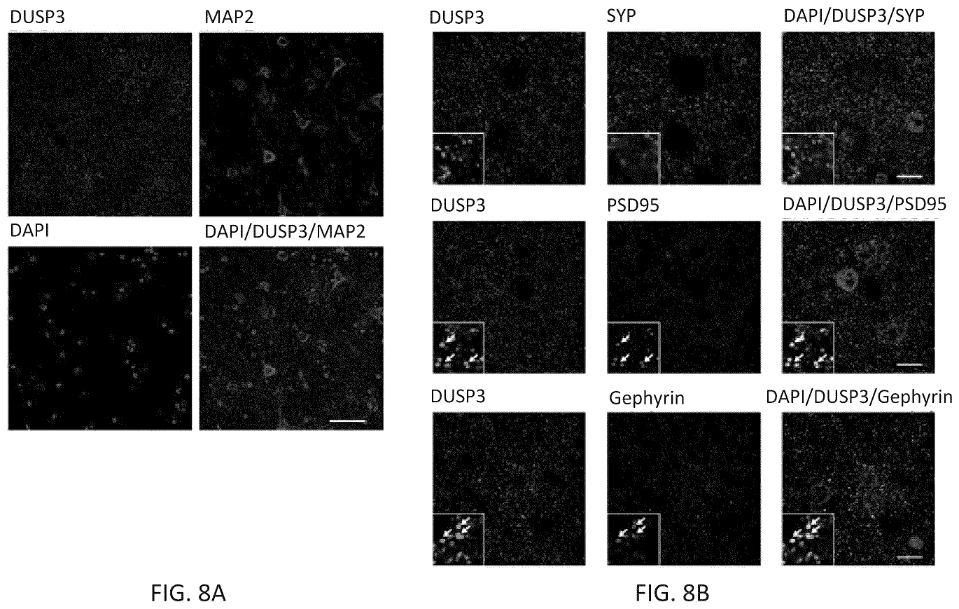

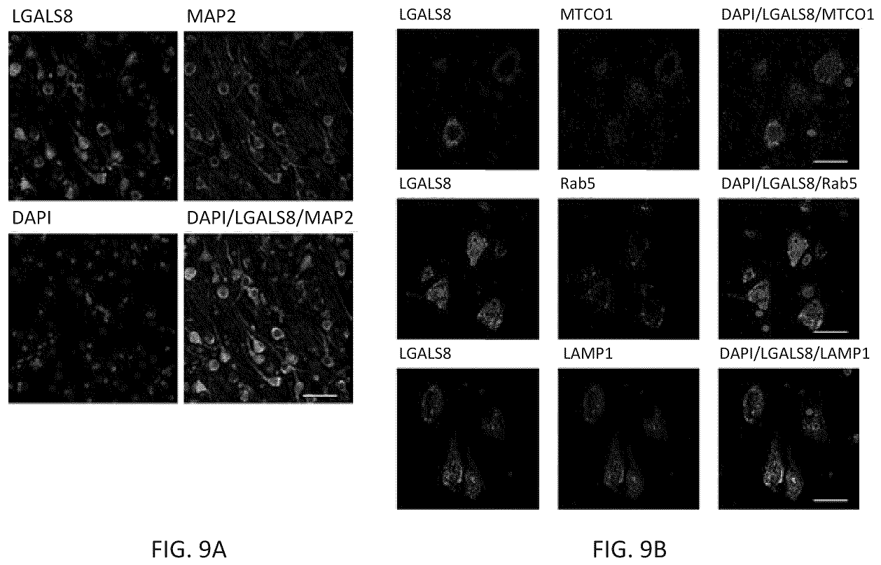

DUSP3 showed positive immunoreactivity with both PSD95 (excitatory postsynaptic marker) and Gephyrin (inhibitory postsynaptic marker), but not with Synaptophysin (presynaptic marker) indicating that the protein is likely located in postsynaptic processes in both excitatory and inhibitory cortical neurons (FIGS. 8A-8B). LGALS8 also showed positive immunoreactivity with LAMP1(a lysosomal marker) rather than MTCO1 (mitochondrial marker) and Rab5 (early endosomal marker) indicating that the protein is likely mainly located in neuronal lysosomes (FIGS. 9A-9B).

### Secondary Validation: External Validation of the Incipient AD Proteomic Signature in Independent Datasets

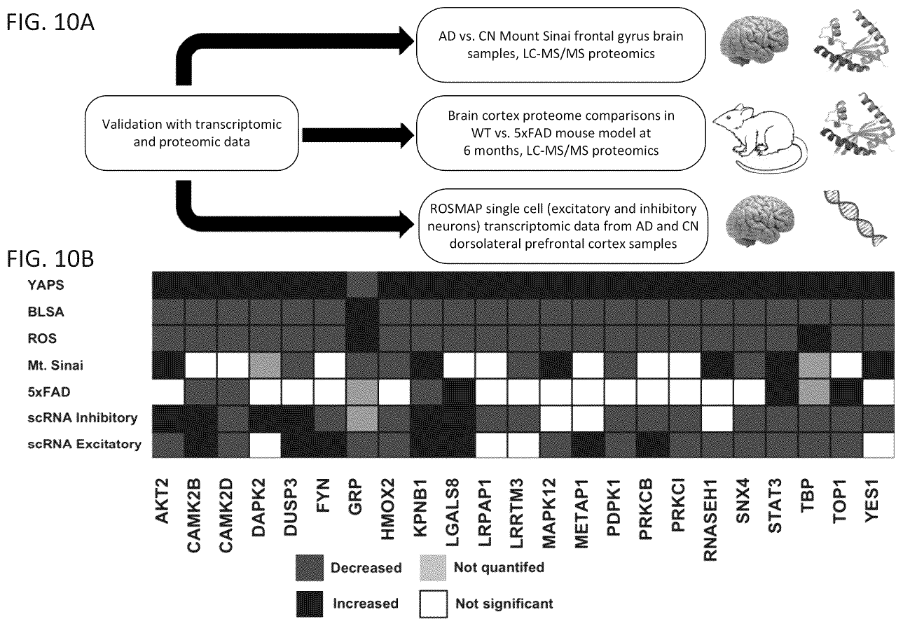

In Step 6b: secondary validation, all 25 proteins included in the incipient AD proteomic signature were assessed using orthogonal methods in independent samples—i.e., mass spectrometry-based proteomics in: 1) AD and CN samples from the Mount Sinai brain bank; 2) 5xFAD transgenic mouse model of AD; and 3) single cell transcriptomics through RNA sequencing (scRNA-seq) from brain samples in the Religious Orders Study and Memory and Aging Project (ROSMAP). The results of these validation analyses are summarized in FIGS. 10A and 10B. Colored squares represent proteins/genes that were differentially abundant/expressed between AD and CN or 5xFAD versus WT mice. Gray squares indicate proteins/transcripts that were not quantified (N.Q.). White squares indicate proteins/transcripts that were not significantly (N.S.) differentially abundant/expressed between AD and CN or 5xFAD versus WT mice. Significance was defined as p<0.05. AD: Alzheimer's disease; BLSA: Baltimore Longitudinal Study of Aging; CN: cognitively normal; ROS: Religious Orders Study; WT: wild type; YAPS: Young APOE Postmortem Study; ROSMAP: Religious Orders Study and Rush Memory and Aging Project; scRNA: single-cell ribonucleic acid; LC-MS/MS: liquid chromatography with tandem mass spectrometry. All 25 proteins validated in at least one of the 3 independent datasets, and 15/25 proteins validated in at least two. Primary data utilized for validation from these three sources were recently published (Bai et al., Neuron 2020, 106:700; Mathys et al., Nature 2019, 570:332-337).

### Mount Sinai Brain Bank

In mass-spectrometry based proteomic data in brain samples from the temporal cortex (Brodmann area 36) collected through the Mount Sinai brain bank, 21 of the 25 proteins in the incipient AD signature were quantified. Twelve of these proteins were differentially abundant in AD versus CN samples (p<0.05).

### 5xFAD Mouse Model

5xFAD mice express human APP and PSEN1 transgenes with five AD-linked mutations: the Swedish (K670N/M671L), Florida (I716V), and London (V717I) mutations in APP, and the M146L and L286V mutations in PSEN1 (Oakley et al., J Neurosci 2006, 26:10129-10140). In mass-spectrometry based proteomic data in brain cortical samples from the 5xFAD transgenic mouse model of AD, 21 of the 25 proteins in the incipient AD signature were quantified. Six of these proteins were differentially abundant in transgenic mice versus WT at 6 months of age (p<0.05).

### ROSMAP scRNA-Seq

In single-cell transcriptomic data from inhibitory and excitatory neurons from the dorsolateral prefrontal cortex samples in the ROSMAP cohort, 22 and 23 of 25 proteins in the incipient AD signature were quantified, respectively. Nineteen (inhibitory neuron cell type) and 19 (excitatory neuron cell type) were differentially expressed in AD versus CN samples (p<0.05).

### Example 3

### Stage 3—Phenotypic Screening of Drugs Targeting the Incipient AD Signature

Among the subset of proteins selected in STAGE 2, drugs targeting the cytokine transducer STAT3 and Src family tyrosine kinases YES1 and FYN were evaluated to determine whether the drugs could rescue molecular phenotypes relevant to AD pathogenesis. To accomplish this, cell culture based phenotypic assays that provided readouts relevant to Aβ secretion/clearance/toxicity, tau pathology, neuroinflammation, and cell death were performed. STAT3, YES1, and FYN were selected as potential AD drug targets and approved/experimental drugs targeting them were chosen as candidate AD treatments. Drugs targeting STAT3 included in these studies were Crizotinib (FDA-approved for non-small cell lung cancer, PZ0191, Sigma-Aldrich) (D'Angelo et al., Cancers (Basel) 2020, 12(11):3293), Napabucasin (FDA-designated orphan drug status for colorectal and gastroesophageal cancer; T3218, Targetmol) (Oncology Times 2016, 38(24):25), and C188-9 (currently in a phase-1 clinical trial of several cancers including lung, hepatocellular and colorectal cancer; ClinicalTrials.gov Identifier: NCT03195699; T4650, Targetmol). Dasatinib (FDA-approved for chronic myeloid leukemia; T1448 Targetmol) was selected as a candidate AD treatment targeting the Src family tyrosine kinases YES1 and FYN (Rousselot et al., Br J Haematol 2021, 194(2):393-402). The drug concentrations are shown in Table 8.

TABLE 8

| | | AB | | Trophic | AB |
|---|---|---|---|---|---|
| | LPS | clearance | Tau | factor | secretion |
| | (μM) | (μM) | (μM) | (μM) | (μM) |
| STAT3 | | | | | |
| Crizotinib | 0.6, 0.3, | 10, 1, 0.1 | 10, 1, 0.1 | 10, 1, 0.1 | 10, 1, 0.1 |
| | 0.1 | | | | |
| Napabucasin | 0.6, 0.3, | 10, 1, 0.1 | 10, 1, 0.1 | 10, 1, 0.1 | 10, 1, 0.1 |
| | 0.1 | | | | |
| C188-9 | 0.6, 0.3, | 10, 1, 0.1 | 10, 1, 0.1 | 10, 1, 0.1 | 10, 1, 0.1 |
| YES1 & | 0.1 | | | | |
| FYN | | | | | |
| Dasatinib | 1, 0.1, | 5, 0.5, | 1, 0.1, | 1, 0.1, | 1, 0.1, |
| | 0.01 | 0.05 | 0.01 | 0.01 | 0.01 |

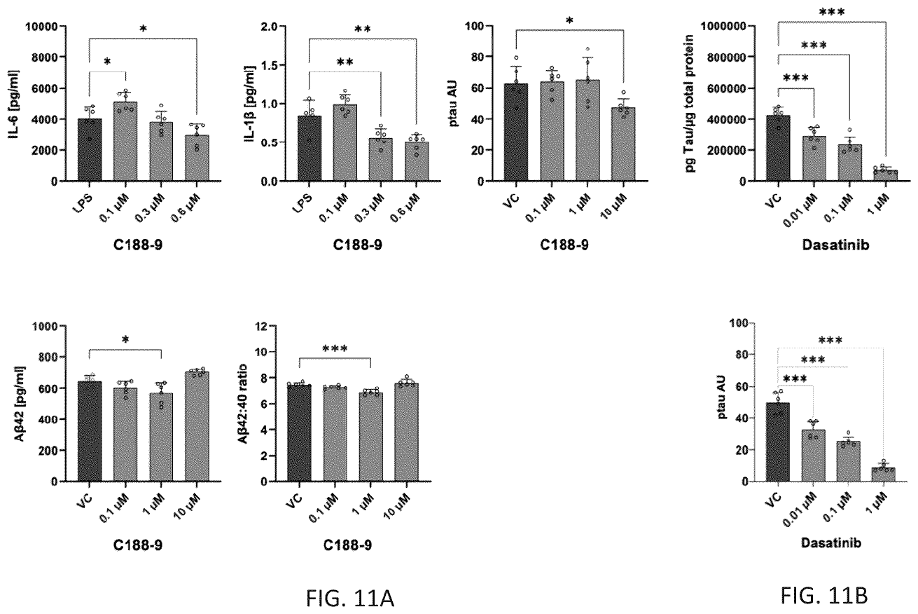

The results are shown in FIGS. 11A-11B. Levels of pro-inflammatory cytokines IL-6 and IL-1β (pg/ml) were measured in the supernatant of BV2 (microglial) cells after 24 h LPS stimulation and assessed using MSD V-plex. Levels of tau (pg tau/μg of total protein) and ptau (AU) were measured in lysates from SH-SY5Y cell line over-expressing mutant human tau441 (SH-SY5Y-TMHT441) after 24 hours of stimulation. Levels of Aβ42 and Aβ40 (pg/ml) were measured in the supernatant of murine BV2 (microglial) cells after 3 h of Aβ stimulation in human APP overexpressing H4 neuroglioma cells. Blue bars indicate the comparison group: either LPS (stimulation to generate proinflammatory cytokines) for LPS-induced neuroinflammation or the VC for tau phosphorylation and Aβ secretion. Orange bars indicate 3 increasing concentrations for treatment with C188-9 or Dasatinib. For the LPS-induced inflammation phenotype, all 3 treatment concentrations were compared to LPS (blue bar); for tau phosphorylation and Aβ secretion all 3 treatment concentrations were compared to the VC (blue bar). Values were compared by one-way ANOVA followed by Dunnetts multiple comparison test and significant differences were indicated (*p<0.05; **p<0.01; ***p<0.001). LPS: lipopolysaccharide; VC: vehicular control (0.1% DMSO); ptau: phosphorylated tau 231; AU: arbitrary units.

As indicated in FIG. 11A, the STAT3 inhibitor C188-9 rescued three AD phenotypes: Lipopolysaccharide (LPS)-induced neuroinflammation, tau phosphorylation, and Aβ secretion. For the bacterial LPS-induced neuroinflammation assay, C188-9 significantly reduced the release of proinflammatory cytokines IL-6 relative to LPS (blue bar) at the highest concentration (0.6 μM), and significantly reduced the release of IL-1β relative to LPS at both 0.3 μM and 0.6 μM concentration in BV2 microglial cells. There were no adverse effects on cell viability and comparisons between LPS and the vehicular control (VC) as well as LPS and reference item dexamethasone (10 μM) (RI dexa) indicate that LPS stimulation successfully generated proinflammatory cytokines (e.g., IL-6 and IL-1β) (results not shown). C188-9 also significantly reduced levels of phosphorylated tau 231 (ptau) relative to the VC (blue bar) at the highest concentration (10 μM). Finally, C188-9 significantly reduced levels of endogenous Aβ1-42 and the Aβ42: Aβ40 ratio relative to the VC (blue bar) at the middle concentration (1 μM) with no adverse effects on cell viability (results not shown) in human APP overexpressing H4 neuroglioma cells.

As indicated in FIG. 11B, the YES1/FYN inhibitor Dasatinib rescued one AD phenotype: tau phosphorylation. Dasatinib significantly reduced levels of total tau and ptau relative to the VC (blue bar) at all 3 concentrations (0.01 μM, 0.1 μM, 1 μM) in the mutant tau441 overexpressing neuroblastoma cell line.

### Discussion of Examples 1-3

In summary, an incipient AD proteomic signature in young APOE ε4 carriers was established to characterize biological alterations in the brain that may precede AD onset by up to three decades. Aptamer-based proteomics was used to first identify a brain proteomic signature of AD in two independent, well-characterized older adult cohorts and then determine that several proteins in this signature were also altered in young APOE ε4 individuals without significant AD pathology. This incipient AD proteomic signature consisted of 25 proteins altered across all three cohorts (BLSA, ROS, and YAPS). A subset of these proteins that are targeted by drugs used for non-AD indications was validated using western blotting in the 3xTg-AD mouse model of AD as well as in AD and CN samples from BLSA. In addition, the subcellular localization of two of these proteins within neuronal lysosomes and post-synaptic processes in excitatory and inhibitory neurons was also confirmed. This signature was then validated in three independent publicly available datasets using orthogonal methods including mass spectrometry-based proteomics in AD and CN samples as well as in the 5xFAD mouse model of AD. Differential neuronal expression of several gene transcripts encoding these proteins in AD was confirmed.

By identifying molecular correlates of APOE ε4+ associated AD risk in young individuals, the results provide a window into very early biological perturbations occurring during the long preclinical phase of AD that may present novel therapeutic targets for disease modification. Evidence supporting this hypothesis was provided by demonstrating that drugs targeting STAT3, YES1 and FYN, three proteins in the incipient AD proteomic signature, reduced neuroinflammation and tau phosphorylation as well as endogenous production of Aβ42 in cell culture based phenotypic assays. Together with recent findings that APOE-targeted immunotherapy reduces brain amyloid deposition and rescues cerebrovascular dysfunction in the 5xFAD mouse model (Xiong et al., Sci Transl Med 2021, 13(581), eabd7522, 12 pages), the results indicate that APOE-associated dysregulation in molecular pathways may offer a promising source of novel drug targets in AD.

Of the 25 proteins altered in young APOE ε4 carriers as well as in AD, some have established roles in both Aβ accumulation as well as tau phosphorylation—molecular events critical to the development of the two primary pathological hallmarks of AD. These include roles both in the amyloidogenic processing of the amyloid precursor protein (APP) as well as in clearance of Aβ from the brain. For example, leucine rich repeat transmembrane neuronal 3 (LRRTM3) is a synaptogenic adhesion molecule involved in synaptic assembly and promotes APP processing by beta-secretase 1 (BACE1) (Schroeder et al., Exp Pol Med 2018, 50:10; Lincoln et al., Plos One 2013, 8:364164; Majercak et al., PNAS USA 2006; 103:17967-17972), while sorting nexin 4 (SNX4) prevents BACE1 trafficking to lysosomes for degradation, thereby facilitating Aβ production (kin et al., Alzheimers Res Ther 2017, 9:4; Toh et al., Mol Biol Cell 2018, 29:191-208). On the other hand, LDL receptor related protein associated protein 1 (LRPAP1) facilitates LRP-mediated Aβ clearance across the blood brain barrier (Sanchez et al., Am J Med Genet 2001, 105:76-78; Van Uden et al., J Neurosci 2002, 22:9298-9304). It is interesting to note that polymorphic variation in both LRRTM3 and LRPAP1 have been associated with increased risk of late-onset AD, further suggesting potential causative roles for these proteins in AD pathogenesis (Reitz et al., Arch Neurol 2012, 69:894-900; Pandey et al., Genes Brain Behav 2008, 7:943-950). Several kinases that participate in multiple signaling cascades relevant to AD in the incipient AD proteomic signature were observed. These include the atypical protein kinase C (aPKC) PKC_{-ι}, Mitogen-Activated Protein Kinase 12 (MAPK12), a member of the p38 MAP kinase family, the Src family tyrosine kinases FYN and YES1 (Anguita et al., Biochim Biophys Acta Mol Cell Res 2017; 1864:915-932), and Ca²⁺/calmodulin (CaM)-dependent protein kinase II (CaMKII), the major post-synaptic protein at excitatory synapses (Lucchesi et al., Brain Res Bull 2011, 85:2-8). PKC_{-ι} has been shown to mediate an increase in BACE activity, Aβ production and tau phosphorylation and is known to be modulated by brain insulin levels (Sajan et al., Neurobiol Aging 2018, 61:226-237). The p38 MAPKs phosphorylate microtubule associated tau in addition to a broad range of proteins and have been shown to be important mediators of the senescence associated secretory phenotype (SASP)—a chronic proinflammatory state in senescent cells, characterized by the secretion of numerous cytokines and chemokines (Freund et al., EMBO J 2011, 30:1536-1548). Prior studies from post-mortem human brains have also reported activation of p38 MAPKs in early stages of AD (Sun et al., Exp Neurol 2003, 183:394-405). Altered protein levels of FYN, an important regulator of pathological tau aggregation and transducer of Aβ signaling suggests that convergent dysregulation of both Aβ and tau-related pathways may be an early feature of AD progression (Briner et al., Cell Rep 2020, 32:108045). The presence of two distinct isoforms of CaMKII in the incipient AD signature is especially interesting given its important roles in synaptic plasticity and tau phosphorylation (Ghosh et al., Mol Brain 2015, 8:78).

The incipient AD signature also contains several proteins with previously unknown roles in AD pathogenesis that may mediate biological actions relevant to AD. These include methionine aminopeptidase 1 (METAP1), which catalyzes removal of N-terminal methionine from newly synthesized proteins and plays an important role in cell cycle progression (Hu et al., PNAS USA 2006, 103:18148-18153); the chemokine CCL19; and IFN lambda (IFN-λ), a member of the interferon family that has been shown to inhibit infection of primary neurons and astrocytes by neurotropic viruses (Li et al., Glia 2011, 59:58-67). Together with altered levels of STAT3, a key signal transducer of cytokine signaling, these findings suggest an early involvement of neuroinflammation in AD progression. The presence of galectin-8 (LGALS8), a beta-galactoside-binding lectin in the incipient AD signature suggests converging pathways between host defense mechanisms against microbial infection and neurodegeneration (Thurston et al., Nature 2012, 482:414-418). Recent evidence also suggests that LGALS8-mediated autophagy is important in preventing the entry of tau seeds into the cytosol and their subsequent aggregation (Falcon et al., J Biol Chem 2018, 293:2438-2451). Our immunohistochemical studies demonstrate that LGALS8 is localized within neuronal lysosomes where it may play a role in autophagic degradation of intraneuronal tau (Jiang et al., Front Mol Neurosci 2020, 13:586731). Our finding that dual-specificity phosphatase 3 (DUSP3), also known as VH1-related phosphatase (VHR) is localized within postsynaptic processes of both excitatory and inhibitory neurons is also consistent with its proposed role in countering excitotoxicity-induced neuronal death and Aβ accumulation (Son et al., Sci Rep 2016, 6:38452).

As seen in FIG. 2D, intriguingly, for nearly all proteins in the incipient AD proteomic signature, the direction of association was opposite between young APOE ε4 carriers and older AD individuals, with most proteins being elevated in young APOE ε4 carriers relative to non-carriers and reduced in AD relative to CN samples. This suggests that alterations in brain proteomic profiles in young APOE ε4 carriers may represent early pathogenic changes in individuals at enhanced risk for AD. Over the next 3 to 4 decades, the functional consequences of these early molecular changes may cause the progressive accumulation of pathology, eventually manifesting as the irreversible cognitive impairment and functional decline characterizing the clinical syndrome of AD. The associations of brain tissue levels of several of the proteins identified with both severity of AD pathology and ante-mortem trajectories of cognitive performance in AD individuals in both BLSA and ROS add further strength to the hypothesis that early perturbations in biological pathways represented by these proteins may be causally associated with AD progression. This hypothesis merits further testing in experimental AD models.

The opposite direction of associations between young APOE ε4 carriers and AD individuals lends itself to multiple interpretations that merit further exploration. This may be consistent with a possible antagonistic pleiotropy effect of APOE ε4, whereby some of the biological changes associated with the ε4 allele confer selective advantages early in life but turn detrimental with age (Tuminello et al., Int J Alzheimers Dis 2011, 726197). Others have suggested that an evolutionary or environmental mismatch may explain the effects of the ε4 allele, as multiple lines of evidence have indicated that APOE ε4 promotes a pro-inflammatory, highly responsive innate immune response (Vitek et al., Neurobiol Aging 2009, 30:1350-1360) that has been shown to protect multiple health indices, including cognition, in regions with high prevalence of infectious disease (Corbett et al., Nat Rev Genet 2018, 19:419-430). Indeed, studies among the Bolivian Tsimane have demonstrated that the APOE ε4 allele protects against cognitive decline only among those with a high parasite burden but contributes to more rapid decline in those without high parasite loads (Trumble et al., FASEB J 2017, 31:1508-1515). Such a mismatch hypothesis appears plausible in the context of the study, in which multiple proteins in the incipient AD signature have defined roles in the innate immune system and related roles in signal transduction.

Although differential expression of individual proteins in brain tissue samples may provide novel insights into AD pathogenesis, the plausible functional consequences of alterations in the biological pathways represented by the measured proteins were also of interest. The GSEA results therefore provided additional biological context to the findings by revealing alterations in numerous molecular pathways in young APOE ε4 carriers relative to non-carriers. Several of these pathways are also altered in AD brain relative to CN samples. Similar to the findings of alterations in individual protein levels described above, the GSEA analyses revealed several biological functions/pathways that appear to be enriched in young APOE ε4 carriers relative to non-carriers (YAPS) and diminished in AD relative to CN individuals (BLSA/ROS). These include signaling cascades involving epidermal growth factor (EGF), insulin, G protein-coupled receptors (GPCRs), diacylglycerol/inositol trisphosphate/calcium (DAG/IP3/Ca²⁺), neurotransmitters, opioids, and peptide hormones. Other enriched modules include endothelial cell function, axon guidance, protein autophosphorylation and humoral immune responses.

Secondary validation of the index results from aptamer-based proteomics within multiple independent samples and orthogonal assays confirmed the findings of alterations in brain protein levels in AD. By comparing aptamer-based brain proteomic findings in BLSA, ROS, and YAPS with deep mass spectrometry-based proteomic profiling results in the Mount Sinai cohort, the results were validated using an independent sample of AD and CN participants. Then using the 5xFAD model, an assessment of alterations in brain protein levels in young APOE ε4 and AD individuals that may be driven by aberrant APP processing (i.e., convergently altered in 5xFAD transgenic mice) and those likely to be independent of Aβ accumulation (i.e., not altered in 5xFAD transgenic mice) was performed. Furthermore, assessment of differentially expressed gene transcripts encoding these proteins within excitatory and inhibitory neurons provided additional functional context to the results. Several of the incipient AD signature proteins were also reported in Johnson et al.'s recent brain proteomic study of AD, of which DUSP3, HMOX2, KPNB1, and STAT3 were also found to be differentially abundant in AD compared to control individuals (Johnson et al., Nat Med 2020, 26(5):769-780).

Accessing the YAPS cohort enabled focus on APOE-related proteomic alterations in young individuals decades prior to the typical age at onset of AD, thereby relating these changes to AD pathogenesis at the early preclinical stages of disease progression. Future studies using unbiased proteomics with a larger coverage of the proteome may uncover additional alterations in other molecular pathways in AD.

Key demographic differences between BLSA and ROS include a predominantly female sample in ROS, whereas less than half of BLSA participants included in this report were female. Prior neuropathological studies in ROS have established that women have a greater global burden of AD pathology and neurofibrillary tangle density in the brain as well as more severe arteriosclerosis (Oveisgharan et al., Acta Neuropathol 2018, 136:887-900). AD participants in the BLSA also had an earlier onset and longer duration of disease compared to those in ROS. These differences may partly explain regional variations in the distribution of protein differences between AD and CN samples in the ITG/MFG across the two cohorts.

Another important consideration is the convergence of the findings across diverse samples, including multiple human autopsy cohorts, two transgenic AD mouse models, and single-cell transcriptomic datasets. While the majority of proteins in the incipient AD signature are also differentially abundant between AD/CN and or Tg/WT protein/transcript samples in at least two independent validation datasets, there were inconsistencies in the direction of observed differences. This likely reflects significant heterogeneity in these models including differences in disease severity and stage of disease progression, as well as limitations of transgenic models in recapitulating all aspects of AD pathogenesis and the complex gene-environment interactions driving AD progression in humans. Similarly, three methodologically distinct approaches were applied to detect and quantify protein levels in this study i.e., aptamer- and mass spectrometry-based proteomics as well as antibody-based western blotting. Fundamental differences in these methods may also contribute to inconsistencies in directionality of some of the observed changes in protein levels. For example, antibody and aptamer-based methods rely upon access to specific epitopes on a given protein for its detection and post-translational modifications at these epitopes may significantly impact the signal arising from these assays. Previous studies comparing consistency of results across these various proteomic platforms have shown that there is only a modest convergence of results from these distinct approaches (Pietzner et al., bioRxiv 2021, 2021.2003.2018.435919; Billing et al., J Proteomics 2017, 150:86-97).

In summary molecular correlates of APOE-related AD risk prior to accumulation of AD pathology and preceding the onset of clinical symptoms were examined. A proteomic study of human brain tissue samples in multiple human cohorts and in two transgenic AD mouse models was performed to identify early protein markers of AD and dysregulation in several biological pathways in APOE ε4 carriers that may be causally related to the eventual accumulation of neuropathology and symptom onset in AD. Some of the proteins implicated include those targeted by existing and experimental drugs in other diseases. Phenotypic screening of drugs targeting proteins in the incipient AD signature indicated that these molecular pathways may represent novel therapeutic targets. These findings may pave the way for future studies to fully understand the biological basis of APOE-associated AD risk and to develop effective interventions targeting the earliest molecular drivers of the disease.

### Example 4

### In Vivo Efficacy Studies in a Transgenic Mouse Model

The disease-modifying effects of candidate drugs in the 5xFAD mouse model for AD are evaluated. 5xFAD (Familiar Alzheimer Disease) mice bear five mutations, three in the amyloid precursor protein (APP695) gene [APP K670N/M671L (Swedish), I716V (Florida), V717I (London)] as well as two mutations in the presenilin 1 gene [PS1 M146L, L286V] (Oakley et al., 2006). The expression of the 5xFAD transgene is driven by the neuron specific Thyl promoter. The five mutations cause an early onset of the cognitive decline and increasing Abeta 1-40 and 1-42 levels in the brain and cerebrospinal fluids, over age. Histological analysis revealed plaque load and beta sheet formation accompanied with neuroinflammation. Thus, the 5xFAD mouse mimics the most crucial phenotypic symptoms of amyloidogenic neurodegeneration, neuroinflammation as well as learning and memory deficits and is a suitable model for Alzheimer's disease to study effects of drugs on biochemical, histological and behavioral hallmarks.

Thirty-two, three months old 5xFAD mice as well as sixteen age- and sex-matched wild-type littermates receive weekly intraperitoneal (IP) injections of a candidate drug for 6 months. After 6 months of treatment, the behavioral effects are tested in the Morris Water Mazer (MWM) and the Y-maze, and terminal samples are collected. The collected samples are then further analyzed using histological and biochemical methods to assess the drug's effects on amyloid and tau pathology, neuronal loss and neuroinflammation.

Thirty-two ˜3 months -old 5xFAD mice are randomly allocated to 2 treatment groups B and C, each consisting of 16 animals. 16 age matched wild type littermates form the non-transgenic vehicle control group A. Even numbers of male and female animals are used in each group.

In-vivo blood samples are collected by mandibular sampling from each animal, at baseline (before treatment start), and after 2, 4 and 5 months of treatment. Maximum allowed blood volume is sampled into K2EDTA (potassium ethylenediaminetetraacetic acid) tubes. Blood plasma is isolated by centrifugation (3000×g for 10 minutes at room temperature) and plasma aliquots are transferred to 1.5 mL tubes, frozen on dry ice and stored at −80° C. Animals receive IP injections of a candidate drug (or vehicle) once per week for 27 weeks. The animals are weighed before each dose and receive the formulation at 10 μL/g body weight.

At the end of the treatment period, all animals are tested once in Morris Water Maze (MWM) and in the Y-maze to assess the test item's effects on the cognitive deficits of the mice. MWM test is performed on 4 days with 4 trials per day and a probe trial on day 5. A computerized video tracking system is used to quantify escape latency and distance travelled, as defined in the according QPS SOP. The tests are performed after treatment. The mice are tested in a randomized order.

Spatial learning capacities of all animals are tested in the Morris Water Maze (MWM). The MWM is performed using the following pattern: four trials on each of four consecutive days are performed. In all trials, the platform is located in the northeast (NE) quadrant of the pool. Mice start from predefined positions (southeast (SE), southwest (SW), northwest (NW)). A single trial lasts for a maximum of 60 seconds. In case the mouse does not find the hidden, diaphanous platform within this time, the experimenter guides the mouse to the target. Mice are allowed to rest on the platform for 10-15 sec to orientate in the surrounding. On day five mice are tested in the probe trial (PT). During the PT, the platform is removed from the pool and the number of crossings over the former target position as well as the abidance in the target quadrant is recorded. For the quantification of escape latency (the time [sec] to find the hidden platform), of pathway (the length of the trajectory [meters] to reach the target), of target zone crossings and of the abidance in the target quadrant in the PT, a computerized video tracking system (Biobserve Viewer III) is used.

Spatial working learning capacities of all animals are tested in the Y-Maze. The Y-Maze apparatus consists of three identical white arms (length×width×height=38×6.5×13 cm). The light will be set to 10 Lux. Each mouse is placed individually in the center of the maze and allowed to move freely through the maze during an 8 minute test session. To avoid odor traces the maze is cleaned with 70% isopropanol between different animals. The process is video recorded and analyzed using Noldus Ethovision software. The sequence of arm entries as well as the number of total arm visits are recorded automatically. An alternation is defined as entering each of the three arms consecutively. The maximum number of alternations is the total number of arms entered minus two. The percentage of alternations is calculated as actual alternations per maximum alternations.

About 24 h after the last treatment, all mice are euthanized by IP injection of 600 mg/kg pentobarbital. CSF is collected from the cisterna magna and snap frozen. Terminal blood is collected by heart puncture in EDTA coated tubes. Blood plasma is collected by centrifugation (3000×g for 10 minutes at room temperature) and plasma aliquots are transferred to 1.5 mL tubes, frozen on dry ice and stored at −80° C. Brains are dissected after transcardial perfusion with saline and hemisected at midline. The left hemi brains are further dissected in hippocampus, cortex, and rest, all parts are weighed and snap frozen on dry ice for biochemical analysis. The right hemi brains are fixed by immersion in freshly prepared 4% paraformaldehyde in PB (pH 7.4) for 2 hours at room temperature. The samples are then transferred to 15% sucrose in PBS and stored at 4° C. until sunk. Hemispheres are then embedded in OCT and frozen in cryomolds with ice-cold isopentane on dry ice, and stored at −80° C.

Twelve levels of the brain are chosen and defined according to the brain atlas of Paxinos and Franklin (“The Mouse Brain in Stereotaxic Coordinates”, 2nd edition, 2001), levels are chosen to collect sections from the cortex and hippocampus. Hemi brains from 6 animals per group (total of 18 hemi brains) dedicated for histological analysis are embedded in OCT medium and 10 μm cryosections are collected. Sections are collected from 12 levels as defined above and 5 sections per level, a total of 60 sections per animal are collected.

Amyloid-β-positive plaques and tau phosphorylation are evaluated using immunofluorescence labeling on a uniform systematic random set of five sections per mouse in a triple labeling/staining experiment:
anti-amyloid fibrils LOC antibody
thioflavin S staining of amyloid plaques
AT8 antibody labeling for pSer202/pThr205 tau
all sections are counterstained with the nuclear dye DAPI

Mosaic images of the stained sections are recorded on a Zeiss automatic microscope AxioScan Z1 with high aperture lenses, equipped with a Zeiss Axiocam 506 mono and a Hitachi 3CCD HV-F202SCL camera and Zeiss ZEN 2.3 software. The target areas are identified by drawing an area of interest (AOI) on the images. A second AOI excludes wrinkles, air bubbles, or any other artifacts interfering with the measurement and defines the area for quantitative image analysis. Background correction is used if necessary and immunoreactive objects are detected by adequate thresholding and morphological filtering (size, shape). Typical readouts are size and intensity of objects, number of objects per mm² (numerical object density), and the percentage of the AOI area that is covered by immunopositive objects. Once the parameters of the targeted objects have been defined in a test run, the quantitative image analysis runs automatically so that the results are operator-independent and fully reproducible.

The frozen hippocampus and cortex samples from 6 animals of each group (total of 36 samples) are homogenized in lysis buffer. An aliquot is taken from each of the homogenates for the analysis of cytokines, the rest of the homogenates is incubated on ice, and centrifuged. The supernatants are collected as the soluble fraction. The insoluble pellets are dissolved in lysis buffer. The resulting homogenate is collected as the insoluble fraction. The levels of ten different cytokines are measured in the 36 aliquots of the brain homogenates using an immunosorbent assay (V-PLEX Custom Mouse Cytokine, Mesoscale Discovery cat.nr. K15048D)) and cytokine levels are statistically evaluated.

Amyloid-β1-40 and amyloid-β1-42 levels are quantified using an MSD-ISA previously established at QPS and according to QPS SOPs. The soluble and insoluble protein fractions from hippocampus and cortex of 6 animals per group (a total of 72 samples) are analyzed for amyloid beta levels. Raw data is statistically evaluated as described below.

Brain extracts (Triton fraction; 36 samples) from section Error! Reference source not found. are analyzed for cytokines (IFN-γ, IL-1β, IL-2, IL-4, IL-5, IL-6, KC/GRO, IL-10, IL-12p70, TNF-α) with a commercially available immunosorbent assay kit (Proinflammatory Panel 1 (mouse) K15048D, Mesoscale Discovery) according to the instructions of the manufacturer and evaluated in comparison to calibration curves provided in the kit.

NfL levels are quantified in 4 in-vivo plasma samples, the terminal plasma sample and CSF samples from 6 animals of each group (total of 108 samples) using the NF-Light® ELISA by UmanDiagnostics AB, Sweden, that has previously been qualified in QPS Austria's lab. Measurements are performed in duplicates, where possible (not possible for CSF) and results are statistically evaluated as described below.

All raw data is analyzed in GraphPad Prism™ 8 (GraphPad Software Inc., USA). Graphs are displayed with group means and standard error of the mean (SEM). In case outliers are detected (using the Grubb's outlier test) they are excluded. Normality distribution of two groups is analyzed by Kolmogorov-Smirnov tests. Depending on Gaussian distribution, unpaired Student's T-test or the nonparametric Mann Whitney test is performed. If more than 2 groups are compared with each other, significance is calculated by One-way or Two-way analysis of variance (ANOVA) followed by the Bonferroni post hoc test. Significance is defined as * p<0.5, **p<0.01 and ***p<0.001.

### Example 5

### Hydroxychloroquine Inactivation of STAT3 in AD-Relevant Cells

### Methods

Cell and neuron cultures: Immortalized human microglia HMC3 cells (ATCC CRL-3304) and astrocytoma 1321N1 cells (Sigma-Aldrich; derived from human brain astrocytoma; Macintyre et al., 1972) were cultured in Eagle's minimum essential medium (EMEM) supplemented with 10% fetal bovine serum (FBS, Gibco) and 1% antibiotics and antimycotics (Gibco). Neuroblastoma SK-N-BE(2)-M17 (M17) (ATCC CRL-2267) cells were cultured in a 1:1 mixture of EMEM and F12 media, supplemented with 10% FBS plus 1% antibiotics and antimycotics.

For primary cultures of embryonic cortical neurons, timed-pregnant mice were obtained from The Jackson Laboratory. Cultures were prepared from embryonic day (E) 14.5 cerebral tissues as described (Zhao et al., Cell Death Differ 2019, 26:1600-1614). Pregnant mice were killed by fast cervical dislocation, embryos and embryo brains were removed, and the cerebral hemisphere was extracted in sterile Hank's balanced saline solution (HBSS). Brain tissues were incubated in 0.25% trypsin-EDTA for 30 min at 37° C.and then transferred to DMEM containing 10% fetal bovine serum (DMEM+). The tissues were transferred to neurobasal (NB) medium containing B27 supplements, 2 mM L-glutamine, antibiotics and antimycotics (Gibco), and 1 mM HEPES and dissociated by trituration using a fire-polished Pasteur pipet. The dissociated cells were seeded into polyethyleneimine-coated plastic culture dishes at a density of 60,000 cells/cm² and cultured in the same B27-containing NB medium. Experiments were started 5 days later.

HCQ treatment: Treatment with hydroxychloroquine Sulfate (Selleckchem, Catalog No.S4430) was performed at 50 μM concentration. This treatment dose did not have adverse effects on cell viability.

Western blot analysis: Protein extracts were obtained by lysing cells with a denaturing buffer containing 2% sodium dodecyl sulfate (SDS) (Sigma-Aldrich) in 50 mM HEPES. After boiling and sonication, whole-cell protein extracts were size-fractionated through polyacrylamide gels and transferred to nitrocellulose membranes (Bio-Rad). Membranes were blocked with 5% non-fat dry milk and immunoblotted. Primary antibodies were employed that recognized total STAT3 (124H6) (Cell Signaling, 9139T), phosphorylated STAT3 (Tyr705) (D3A7) XP® (Cell Signaling, 9145S), and phosphorylated STAT3 (Ser727) (Cell Signaling, 94994S).

Statistical analysis: Digitized images were obtained, processed, and quantified with ImageLab version 6.1 (BioRad Laboratories).

### Results

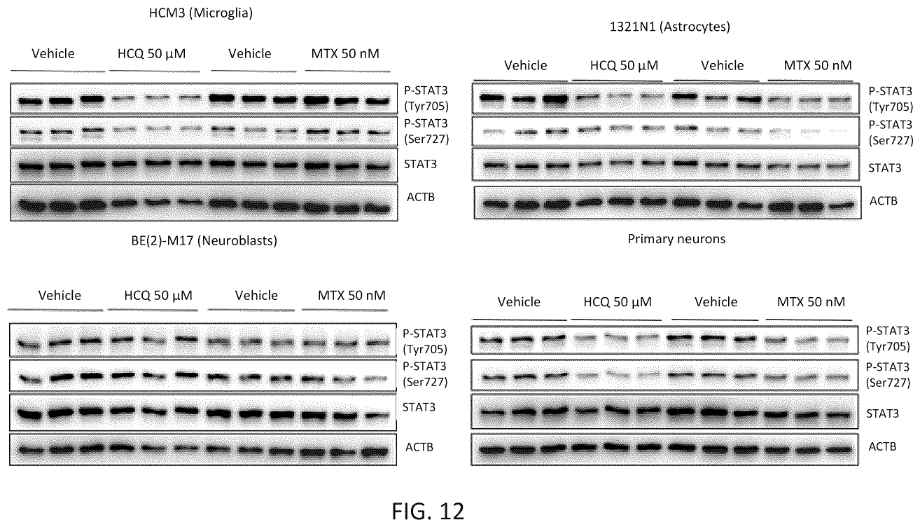

To evaluate whether HCQ is a STAT3-inactivator in cell lines relevant to Alzheimer's disease, western blotting was used to determine whether HCQ treatment (50 μM) alters levels of phosphorylated STAT3 and total STAT3 in human microglia (HMC3 cells), astrocytoma (1321N1 cells), neuroblastoma (SK-N-BE(2)-M17), and mouse embryonic cortical neurons. After 48 hours of HCQ treatment, levels of p-STAT3 (Tyr705 and Ser727) were reduced without alteration in total STAT3 levels in HMC3 cells, 132N1cells and mouse cortical neurons (FIG. 12). Results were non-significant for SK-N-BE(2)-M17 cells.

### Example 6

### Effect of HCQ on AD-Related Phenotypes in Cell Cultures

The ability of HCQ to rescue molecular phenotypes relevant to AD including Aβ₁₋₄₂ clearance, Aβ secretion, Aβ toxicity, tau phosphorylation, lipopolysaccharide (LPS)-induced neuroinflammation, cell death due to trophic factor withdrawal and neurite outgrowth was investigated.

### Methods

Aβ₁₋₄₂ clearance: For Abeta (Aβ₁₋₄₂) clearance assay, 20,000 BV-2 cells per well (uncoated 96 well plates) were plated out. After changing cells to treatment medium, HCQ (0.25 μM, 2.5 μM and 25 μM) was applied 1 hour before Aβ₁₋₄₂ stimulation (Bachem 4061966; final concentration in well: 200 ng/ml (dilutions in medium)). Cells treated with vehicle (0.1% DMSO) and cells treated with Aβ₁₋₄₂ alone served as controls. After 3 h of Aβ₁₋₄₂ stimulation, cell supernatants were collected for the Aβ₁₋₄₂ measurement and cells were carefully washed twice with PBS and thereafter lysed in 35 μL cell lysis buffer (50 mM Tris-HCl, pH 7.4, 150 mM NaCl, 5 mM EDTA, 1% SDS) supplemented with protease inhibitors. Supernatants and cell lysates were analyzed for human Aβ₁₋₄₂ with MSD® V-PLEX Human Aβ42 Peptide (6E10) Kit (K151LBE, Mesoscale Discovery). The immune assay was carried out according to the manufacturer's manual and plates were read on the MESO QuickPlex SQ 120.

Aβ₁₋₄₂ clearance-Protonex assay: For Abeta (Aβ₁₋₄₂) clearance Protonex assay, 5000 BV-2 cells per well (uncoated 96 well plates) were plated out. After changing cells to treatment medium, HCQ (25 μM) was applied 1 hour before stimulation with Protonex™ Green 500, SE (21216, AAT Bioquest) labelled Aβ₁₋₄₂ (Bachem 4061966; final concentration in well: 200 ng/ml (dilutions in medium)). Cells treated with vehicle and cells treated with Aβ₁₋₄₂ alone served as controls. After 3 h of Aβ₁₋₄₂ stimulation, cells were imaged on Cytation 5 multimode reader (Biotek) and green fluorescence was measured.

Aβ secretion: Human APP overexpressing H4-hAPP cells were cultivated in Opti-MEM supplemented with 10% FCS, 1% penicillin/streptomycin 200 μg/mL Hygromycin B and 2.5 μg/mL Blasticidin S. H4-hAPP cells were seeded into 96 well plates (2×10⁴ cells per well). On the next day, cells in 96 well plates were treated HCQ (0.25 μM, 2.5 μM and 25 μM) or the reference item (DAPT 400 nM), a γ-secretase inhibitor vehicle. 24 h later, supernatants were collected for further Aβ measurements by MSD® (V-PLEX Aβ Peptide Panel 1 (6E10) Kit, K15200E, Mesoscale Discovery).

Aβ toxicity: Primary hippocampal neurons were prepared from E18.5 timed pregnant C57BL/6JRccHsd mice as previously described. Cells were seeded in poly-D-lysine pre-coated 96-well plates at a density of 4×104 cells/well and cultivated until DIV10 (Neurobasal, 2% B-27, 0.5 mM glutamine, 25 μM glutamate, 1% Penicillin-Streptomycin). On DIV10 pre-aggregated Aβ₁₋₄₂ (Bachem 4061966, final concentration 10 μM, 48 h at 4° C.) was added to the cells in the presence or absence of Hydroxychloroquine sulfate (TargetMol, T0951) at 25 μM, 2.5 μM or 0.25 μM concentrations. On DIV16 cells were subject to MTT assay to determine cell viability.

Tau phosphorylation: SH-SY5Y-hTau441(V337M/R406W) cells were maintained in culture medium (DMEM medium, 10% FCS, 1% NEAA, 1% L-Glutamine, 100 μg/mL Gentamycin, 300 μg/mL Geneticin G-418) and differentiated with 10 μM retinoic acid (RA) for 5 days changing medium every 2 to 3 days. Prior to the treatment, cells were seeded onto 24-well plates at a cell density of 2×10⁵ cells per well (DIV1). HCQ (25 μM) was applied on DIV2. After 24 h of incubation (DIV3), cells on 24-well plates were harvested in 60 μL RIPA-Buffer [50 mM Tris pH 7.4, 1% Nonidet P40, 0.25% Na-deoxy-cholate, 150 mM NaCl, 1 mM EDTA supplemented with freshly added 1 μM NaF, 0.2 mM Na-ortho-vanadate, 80 μM Glycerophosphate, protease (Calbiochem) and phosphatase (Sigma) inhibitor cocktail]. Protein concentration was determined by BCA assay (Pierce, ThermoFisher) and samples were adjusted to a uniform total protein concentration. Total Tau and phosphorylated Tau were determined by immunosorbent assay from Mesoscale Discovery (Phospho(Thr231)/Total Tau Kit K15121D, Mesoscale Discovery).

Lipopolysaccharide (LPS)-induced neuroinflammation: The murine microglial cell line BV-2 was cultivated in DMEM medium supplemented with 10% FCS, 1% penicillin/streptomycin and 2 mM L-glutamine (culture medium). For LPS stimulation assay, 5000 BV-2 cells per well (uncoated 96 well plates) were plated out and the medium was changed to treatment medium (DMEM, 5% FCS, 2 mM L-glutamine). After changing cells to treatment medium, HCQ (0.25 μM, 2.5 μM and 25 μM) was applied 1 hour before LPS stimulation (Sigma-Aldrich; L6529; 1 mg/ml stock in ddH₂O, final concentration in well: 100 ng/ml (dilutions in medium)). Cells treated with vehicle, cells treated with LPS alone, as well as cells treated with LPS plus reference item (dexamethasone 10 μM, Sigma D4902) served as controls. After 24 h of stimulation, cell supernatants were collected for the cytokine measurement (V-PLEX Proinflammatory Panel 1 Mouse Kit, K15048D, Mesoscale) and cells were subjected to MTT assay.

Cell death due to trophic factor withdrawal: Primary cortical neurons from E18 C57Bl/6 mice were prepared as previously described. On the day of preparation (DIV1), cortical neurons were seeded on poly-D-lysine pre-coated 96-well plates at a density of 3×10⁴ cells per well. Every 4-6 days, a half medium exchange using full medium (Neurobasal, 2% B-27, 0.5 mM glutamine, 1% Penicillin-Streptomycin) was carried out. On DIV8, a full medium exchange to B-27 free medium (Neurobasal, 0.5 mM glutamine, 1% Penicillin-Streptomycin) was performed and HCQ (0.25 μM, 2.5 μM and 25 μM) was applied thereafter. The experiment was carried out with n=6 technical replicates per condition, vehicle treated cells served as control. After 28 h on B-27 free medium, cells were subject to YOPRO/PI and MTT as well as LDH assay.

YO-PRO™-1 (Invitrogen; Y3603) assay was carried out to detect apoptotic cells in combination with Propidium iodide (PI; P4864 Sigma Aldrich) staining for necrotic cells. Part of the supernatant of the cultivated cells was sucked off, so that 90 μL remained per well. 50 μM YO-PRO 1 solution was prepared out of the 1 mM YO-PRO 1 stock solution in DMSO. The stock solution was diluted in a ratio of 1:20 in PBS and Propidium iodide (PI) was added to the same stock to a final concentration of 1 μg/mL. 10 μL of this 50 μM YO-PRO 1/1μg/mL PI solution in PBS was added to the remaining 90 μL to result in a final concentration of 5 μM YO-PRO 1 in the well. Incubation for 15 min in the incubator at 37° C. was performed (light protected). Supernatant was aspirated completely and discarded. 140 μL PBS was added to well. Plate was measured at the multimode-reader (Cytation 5, BioTek).

MTT solution was added to each well in a final concentration of 0.5 mg/mL. After 2 h, the MTT containing medium was aspirated. Cells were lysed in 3% SDS and the formazan crystals were dissolved in isopropanol/HCl. Optical density was measured with a Cytation 5 (Biotek) multimode reader at wavelength 570 nm. Values were calculated as percent of control values (vehicle control or lesion control).

The Lactate dehydrogenase (LDH) toxicity assay was carried out on the supernatants collected after treatment using the Cytotoxicity Detection Kit (Roche Diagnostics, Cat. No: 11 644 793 001). 70 μL of cell culture supernatant was transferred to clear 96-well plates. 70 μL freshly prepared reaction mixture was added to each well and the mixture was incubated for 1 h at room temperature protected from light. Absorbance was measured at 492 nm and 620 nm as reference wavelength with a Cytation 5 (Biotek) multimode reader. Values of culture medium were subtracted as background control. Values were calculated as percent of control values (vehicle control or lesion control).

Neurite outgrowth and neurogenesis: Primary hippocampal neurons were prepared from E18.5 timed pregnant C57BL/6JRccHsd mice as previously described. Cells were seeded in poly-D-lysine pre-coated 96-well plates at a density of 2.6×104 cells/well in (Neurobasal, 2% B-27, 0.5 mM glutamine, 25 μM glutamate, 1% Penicillin-Streptomycin). Directly on DIV1 HCQ (TargetMol, T0951) at 25 μM, 2.5 μM or 0.25 μM concentrations or vehicle control was applied. On DIV2 10 μM Bromodeoxyuridine (BrdU; B5002 Sigma Aldrich) was added and cells were fixed after additional 24 h. Cells were permeabilized with 0.1% Triton-X and incubated with primary Beta Tubulin Isotype III (T8660, Sigma Aldrich) and BrdU antibodies (MAS250c, AHrlan-Sera Lab) overnight at 4° C. Afterwards cells were washed two times with PBS and incubated with fluorescently labeled secondary antibodies and DAPI for 1.5 hour at RT in the darkness. Cells were rinsed three times with PBS imaged with the Cytation 5 Multimode reader (BioTek) at 10× magnification (6 images per well). BrdU positive cells were counted as marker for neurogenesis and Beta Tubulin Isotype III signal was used for macro-based quantification of neurite outgrowth.

Statistical analyses: Statistical analysis was performed in GraphPad Prism 9.1.2. Group differences were evaluated for each test item separately by One-way ANOVA followed by Dunnett's multiple comparison test versus vehicle or lesion control.

Western blot analyses: Samples for western blot analyses were prepared from 3 assays which provided evidence of HCQ-associated rescue (i.e., Ab₁₋₄₂ clearance, tau phosphorylation, and LPS-induced neuroinflammation). Samples were prepared identically as described above in section: Effect of HCQ on AD-related phenotypes in cell culture, and scaled up to 24-well format to allow for adequate sample volume. After HCQ treatment, cells were washed once with PBS and harvested in 50 μl 50 mM HEPES+2% SDS lysis buffer. The extracts were then boiled at 95° C. for 5 mins. Analytic methods were identical to those described above in Example 4.

Statistical analyses: Statistical analysis was performed using STATA 16.1. Group differences were evaluated using two-sample t-tests (parametric) to calculate differences in p-STAT3 levels between HCQ treated and control samples. We additionally used the Wilcoxon rank-sum test (non-parametric) to confirm that results were robust to distributional assumptions. Significant differences were indicated as p<0.05.

### Results

To test whether HCQ rescues molecular outcomes relevant to AD in cell culture-based phenotypic assays, its effects on Aβ₁₋₄₂ clearance, secretion, and toxicity, tau phosphorylation, lipopolysaccharide (LPS)-induced neuroinflammation, cell death due to trophic factor withdrawal and neurite outgrowth and neurogenesis were evaluated.

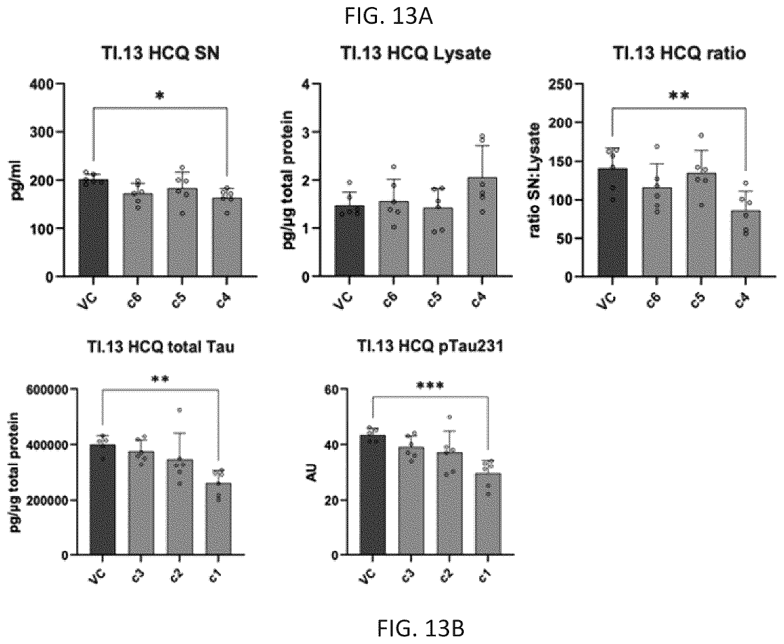

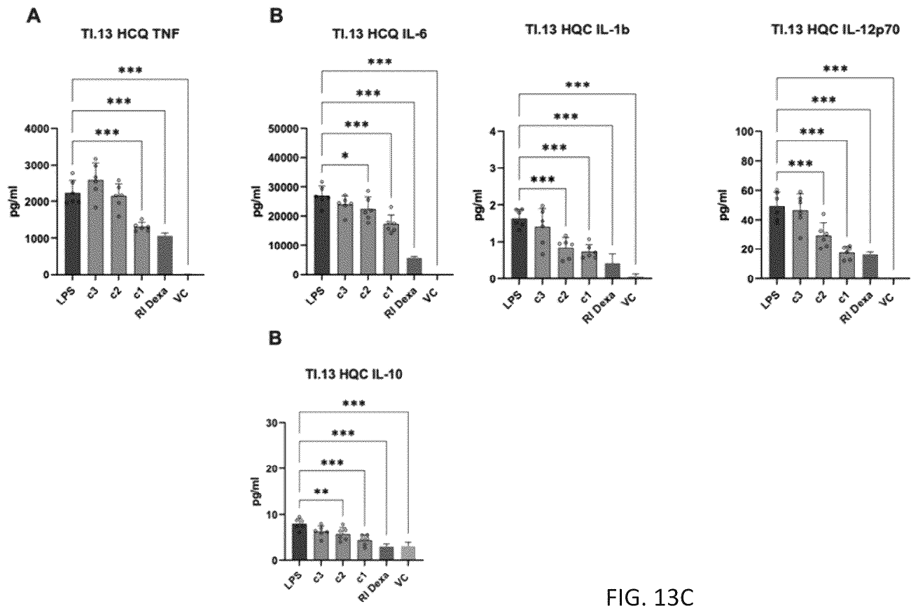

In murine BV-2 microglial cells, HCQ (25 μM) increased Aβ₁₋₄₂ clearance as shown by reduced levels of Aβ₁₋₄₂ in the supernatant and a lowering of the Aβ₁₋₄₂ supernatant:lysate ratio (i.e. phagocytized Aβ₁₋₄₂ in cells) without adverse effects on cell viability (FIG. 13A). HCQ (25 μM) reduced levels of total tau and phosphorylated tau (ptau231) in SH-SY5Y cells overexpressing human mutant tau-hTau441(V337M/R406W) (FIG. 13B). In the murine BV-2 microglial cell line following stimulation with LPS, HCQ (2.5 μM, 25 μM) reduced levels of secreted pro-neuroinflammatory cytokines in a dose dependent manner. A reduction in TNF-alpha secretion was observed at the highest HCQ concentration (25 μM) and a dose dependent (2.5 μM<25 μM) reduction in IL-6, IL-1b, IL-12p70 and IL-10 occurred without any adverse effects on cell viability (FIG. 13C).

In human APP over-expressing H4 neuroglioma cells, HCQ did not alter levels of secreted Aβ₁₋₄₂, and had no effects on toxicity due to exogenous Aβ in primary hippocampal neurons. No effects of HCQ on cell death were observed in response to trophic factor withdrawal in primary cortical neurons, or neurite outgrowth and neurogenesis in primary hippocampal neurons.

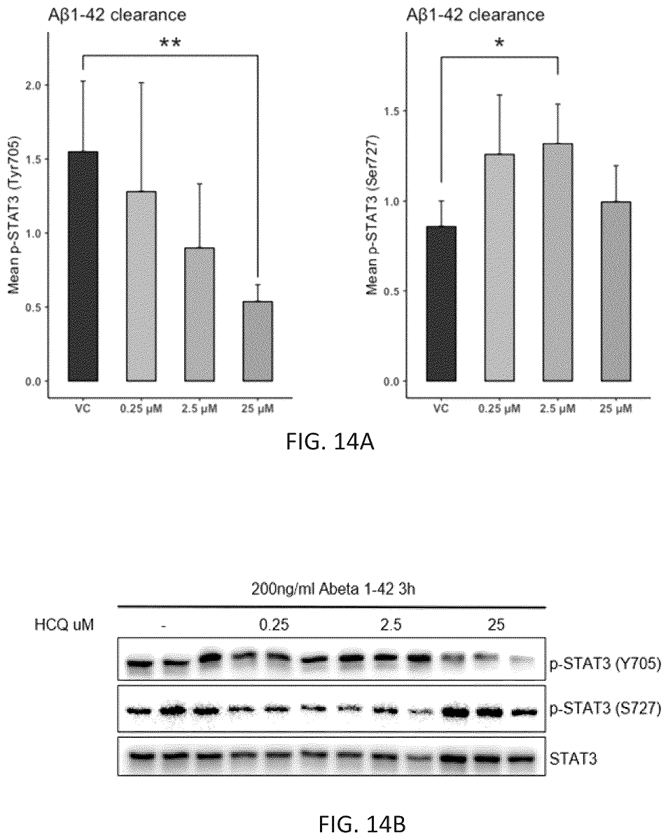

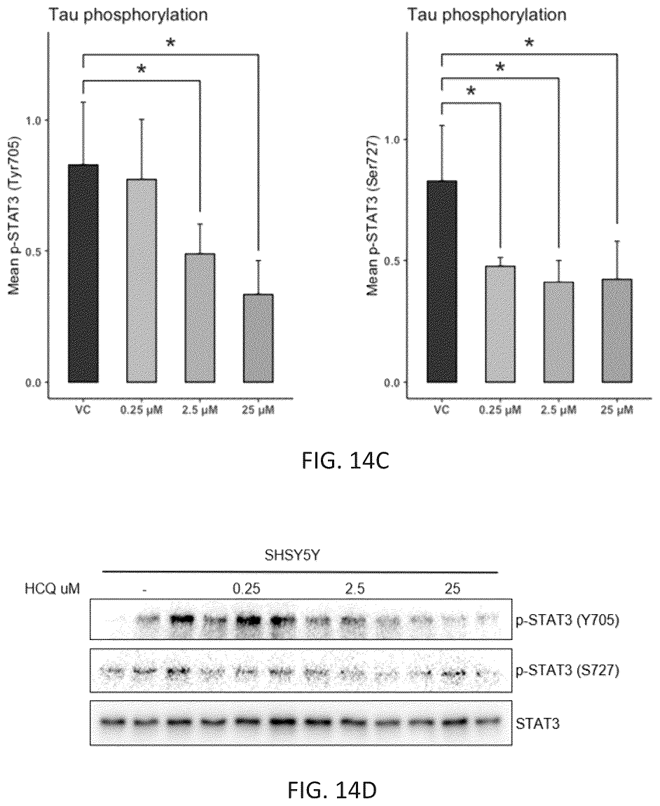

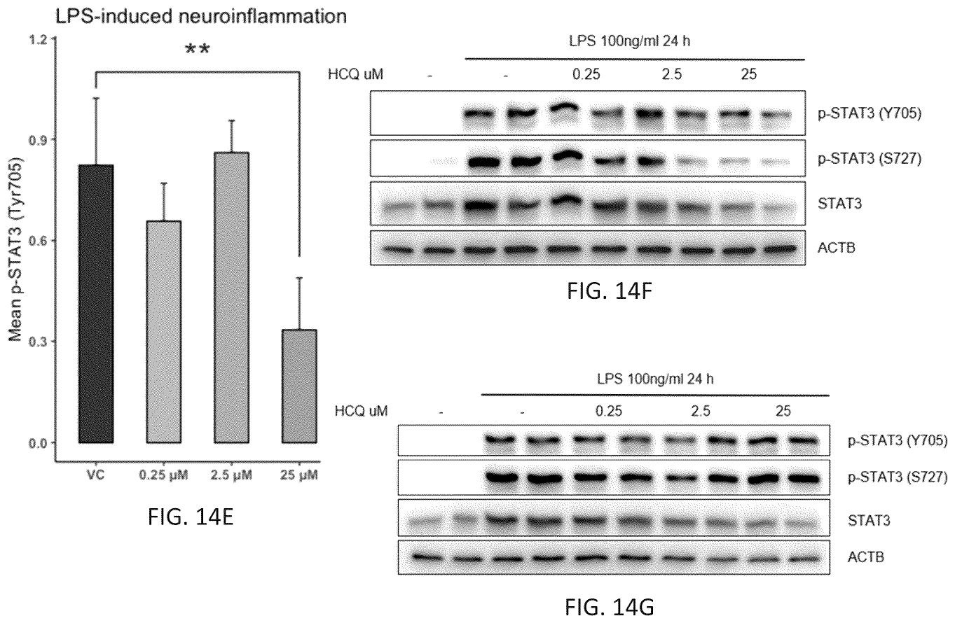

To test whether HCQ-related amelioration of molecular abnormalities relevant to AD was associated with inactivation of STAT3, levels of total and phosphorylated STAT3 were examined to determine whether they were reduced by HCQ treatment in the phenotypic assays of Aβ₁₋₄₂ clearance, tau phosphorylation, and LPS-induced neuroinflammation. Cell homogenates from each of these assays were harvested and tested to determine whether levels of total STAT3 and phosphorylated STAT3 (Tyr705 and Ser727 epitopes) differed between HCQ-treated cells and the relevant vehicular control (VC) condition. As shown in FIGS. 14A-14B, western blot results, and the accompanying graphs, showed that in the Aβ₁₋₄₂ clearance assay, the highest concentration of HCQ (25 μM) lowered p-STAT3 (Tyr705) relative to vehicular control (VC). In the tau phosphorylation assay (FIGS. 14C-14D), HCQ lowered p-STAT3 (Tyr705) levels relative to VC at 25 μM and 2.5 μM concentrations, and p-STAT3 (Ser727) levels at 25 μM, 2.5 μM, and 0.25 μM concentrations. In the LPS-induced neuroinflammation assay (FIGS. 14E-G), p-STAT3 (Tyr705) was significantly lower than the VC at the highest HCQ concentration (25 μM).

### Example 7

### HCQ Rescue of Impaired Long-Term Potentiation in the Hippocampus

### Methods

Animals: All animal experiments and procedures were approved by the Institutional Animal Care and Use Committee (IACUC) of National University of Singapore. We used a transgenic mouse model of AD, which expresses a mutated chimeric mouse/human APP and the exon-9-deleted variant of human PS1, both linked to familial AD, under the control of a prion promoter element (APPSwe/PS1dE9), which we denote as APP/PS1 (Borchelt et al., Neuron 1997, 19(4):939-45). A total of 35 hippocampal slices (21 APP/PS1 and 14 WT) were prepared from 9 APP/PS1 mice and 7 WT mice. Animals were housed under 12 h light/12 h dark conditions with food and water available ad libitum.

Hippocampal slice preparation: Animals were anaesthetized briefly using CO₂ and were decapitated. Brains were quickly removed in 4° C. artificial cerebrospinal fluid (aCSF), a modified Krebs-Ringer solution containing the following (in mM): 124 NaCl, 3.7 KCl, 1.2 KH₂PO₄, 1 MgSO₄.7H₂O, 2.5 CaCl₂.2H₂O, 24.6 NaHCO₃, and 10 D-glucose. The pH of aCSF was between 7.3 and 7.4 when bubbled with 95% oxygen and 5% carbon dioxide (carbogen). Both right and left hippocampi were dissected out in cold (2-4° C.) aCSF, which was being continuously bubbled with carbogen. Transverse hippocampal slices of 400 μm thickness were prepared from the right and left hippocampus using a manual tissue chopper (Stoelting, Wood Dale, Illinois), and transferred onto a nylon net placed in an interface chamber (Scientific Systems Design, Ontario, Canada) and incubated at 32° C. at an aCSF flow rate of 1 ml/min and carbogen consumption of 16 l/h. The entire process of animal dissection, hippocampal slice preparation and placement of slices on the chamber was done within approximately five minutes to ensure that hippocampal slices were in good condition for electrophysiology studies. The slices were incubated for at least 3 h before starting the experiments.

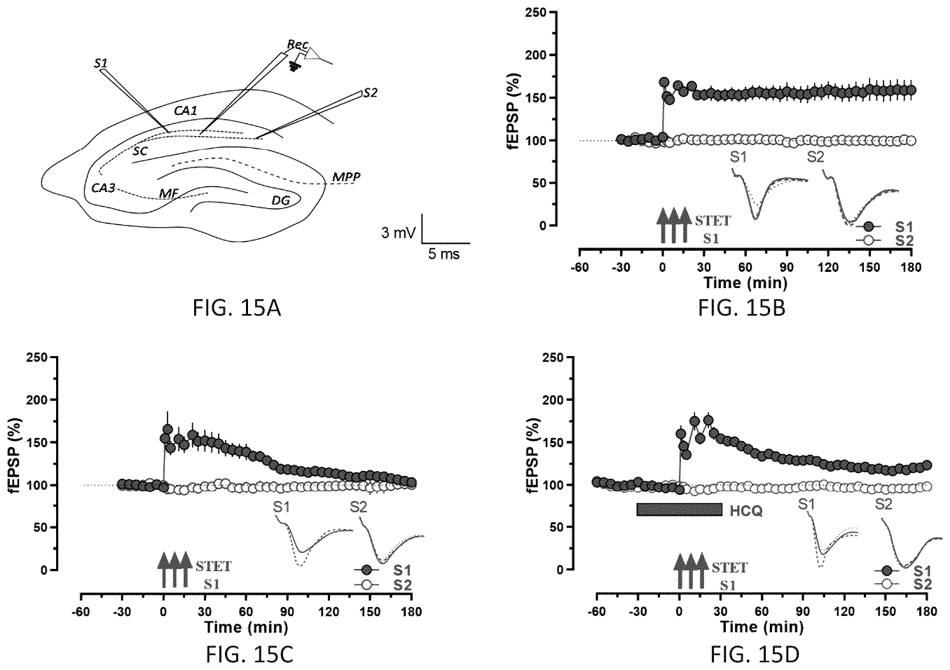

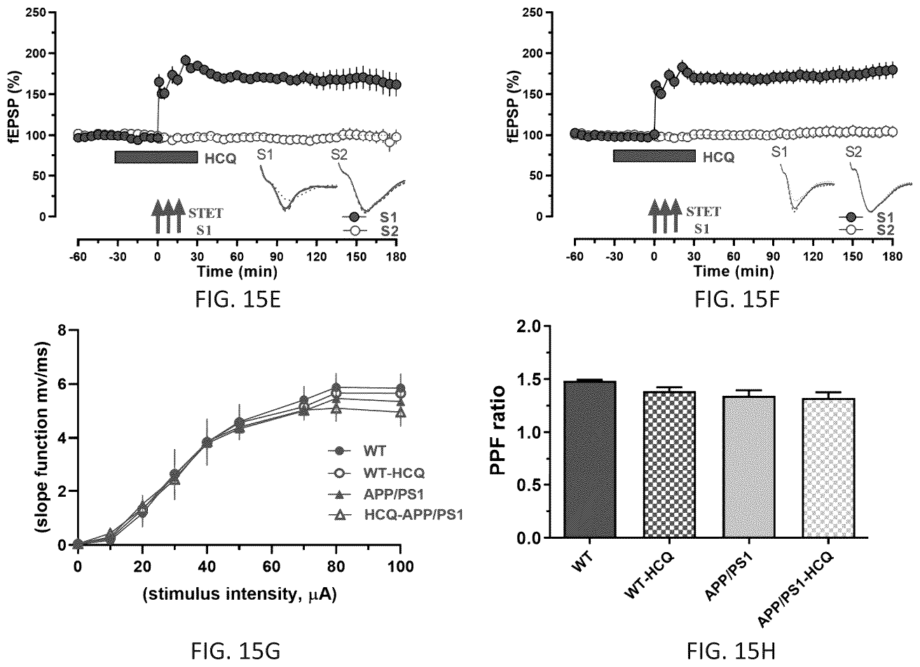

Field potential recordings: In all the electrophysiology recordings, two-pathway experiments were performed. Two monopolar lacquer-coated stainless-steel electrodes (5MΩ; AM Systems, Sequim) were positioned at an adequate distance within the stratum radiatum of the CA1 region for stimulating two independent synaptic inputs S1 and S2 of one neuronal population, thus evoking field excitatory postsynaptic potentials (fEPSP) from Schaffer collateral/commissural-CA1 synapses (FIG. 15A). One electrode (5MΩ; AM Systems) represented as ‘rec’ was placed in the CA1 apical dendritic layer for recording fEPSP. After the pre-incubation period, a synaptic input-output curve (afferent stimulation vs. fEPSP slope) was generated. Test stimulation intensity was adjusted to elicit fEPSP slope of 40% of the maximal slope response for both synaptic inputs S1 and S2. The signals were amplified by a differential amplifier, digitized using a CED 1401 analog-to-digital converter (Cambridge Electronic Design, Cambridge, UK) and monitored online with custom-made software. To induce late long-term potentiation (LTP), a “strong” tetanization (STET) protocol consisting of three trains of 100 pulses at 100 Hz (single burst, stimulus duration of 0.2 ms per polarity), with an inter-train interval of 10 min, was used. In all experiments, a stable baseline was recorded for at least 30 min using four 0.2-Hz biphasic constant-current pulses (0.1 ms per polarity) at each time point. Four 0.2-Hz biphasic, constant current pulses (spaced at 5 s) given every five minutes were used for post-induction recordings also and the average slope values from the four sweeps was considered as one repeat while used for plotting. Initial slopes of fEPSPs were expressed as percentages of baseline averages. A series of pulses ranging from 0, 10, 20, 30, 40, 50, 70, 100 microamperes were applied to generate an input output curve. Graphs are plot as stimulus intensity versus fEPSP slope. Paired pulse ratio (PPR) was evoked using an interstimulus interval of 50 ms at 40% of maximum stimulus intensities. PPR was expressed as the ratio of the fEPSP slope of second stimulus to the first stimulus.

Pharmacology: Hydroxychloroquine sulphate (HCQ) (Selleckchem, catalog, No-S4430) was stored at −20° C. as 50 mM stock in deionized water. Before application, the stock solution was diluted to a final concentration of 25 μM or 50 μM in aCSF and bath-applied for a total of 60 min, 30 min before and 30 min after the STET or unless otherwise specified.

Statistical analysis: All data are represented as mean±SEM. The fEPSP slope value expressed as percentages of average baseline values per time point was subjected to statistical analysis using Graph Pad Prism (Graph Pad, San Diego, CA, USA). Wilcoxon signed rank test (Wilcox test) was used to compare fEPSP values within one group and Mann-Whitney U test (U test) was used when data were compared between groups. Statistical comparisons for input-output (I/O) curve and paired pulse facilitation (PPF) experiments were performed using two-way ANOVA test. P<0.05 was considered as the cutoff for statistically significant differences.

### Results

To test whether HCQ may rescue impaired hippocampal synaptic plasticity in AD, its effects on late-long term potentiation (L-LTP), a form of synaptic plasticity that has been shown to be impaired before the accumulation of AD pathology in the APP/PSI transgenic mouse model of AD, were studied. FIG. 15A illustrates the location of electrodes in hippocampal slice preparations from APP/PS1 transgenic and WT mice (age 12 weeks). As a control, using hippocampal slices from WT mice, late-LTP was first recorded by applying strong tetanization (STET; S1 electrode) the, following the recording of a stable baseline for 30 min (FIG. 15B). This resulted in a long-lasting and stable late-LTP during a recorded time period of 180 min (FIG. 15B: filled blue circles). The control input remained stable throughout the recording time period. (FIG. 15B: open blue circles). Statistically significant differences were observed from 1 min until 180 min when compared with its own baseline and with control input S2 (1 min Wilcox, P=0.01, 1 min U-test, P=0.0006; 60 min Wilcox, P=0.01, 60 min U-test, P=0.0006; 120 min Wilcox, P=0.01, 120 min U-test, P=0.0006; 180 min Wilcox, P=0.01, 180 min U-test, P=0.0006).

Next, the same experimental paradigm was used to study late-LTP in hippocampal slices from APP/PS1 mice. As indicated in FIG. 15C, applying STET to S1 resulted in only short-lasting form of LTP (early-LTP) (FIG. 15C: filled circles) while the control input S2 remained stable (FIG. 15C: open circles). Significant difference was observed only until 120 min (175 min Wilcox, P=0.15, 120 min U-test, P=0.09).

The effect of HCQ (25 μM applied before and after induction of STET) was then tested on late-LTP in hippocampal slices from APP/PS1 mice. As shown in FIG. 15D, bath application of HCQ induced late-LTP which was significantly different from 1 min up to 180 min when compared to its own baseline and S2 (1 min Wilcox, P=0.007, 1 min U-test, P=0.0002; 60 min Wilcox, P=0.007, 60 min U-test, P=0.0002; 120 min Wilcox, P=0.007, 120 min U-test, P=0.0003; 180 min Wilcox, P=0.01, 180 min U-test, P=0.01). At this concentration, HCQ (25 μM) induced late-LTP in APP/PS1 hippocampus that was similar in magnitude to wild type up to 60 minutes (i.e. no statistically significant differences between L-LTP in Tg versus WT; 1 min Wilcox, P=X, 1 min U-test, P=X; 60 min Wilcox, P=X, 60 min U-test, P=X) (FIG. 15D vs FIG. 15B). From 70 minutes through 180 minutes, L-LTP in APP/PS1 hippocampus after HCQ application (25 μM) was significantly lower than in WT (70 min, U-test, P=0.009; 180 min, U-test, P=0.01) (FIG. 15D vs FIG. 15B), suggesting that HCQ at 25 μM partially rescued impaired late-LTP in APP/PS1 mice.

Finally, a higher concentration of HCQ (50 μM applied before and after induction of STET) was tested on late-LTP in hippocampal slices from APP/PS1 mice. As indicated in FIG. 15E, HCQ induced late-LTP (S1)(filled circles) that was significantly different when compared to its own baseline and S2 (open blue circles) up to 180 min (1 min Wilcox, P=0.01, 1 min U-test, P=0.0006; 60 min Wilcox, P=0.01, 60 min U-test, P=0.0006; 120 min Wilcox, P=0.01, 120 min U-test, P=0.0006; 180 min Wilcox, P=0.01, 180 min U-test, P=0.004). At this higher concentration, HCQ-induced late-LTP in APP/PS1 hippocampus was similar in magnitude to WT late-LTP throughout the recording duration of 180 minutes ((i.e. no statistically significant differences between L-LTP in Tg versus WT; 1 min U-test, P=0.62; 60 min U-test, P=0.07; 120 min U-test, P=0.62; 180 min U-test, P=>0.99) (FIG. 15E vs FIG. 15B), suggesting that HCQ at 50 μM fully rescued impaired late-LTP.

Comparison of input-output (I/O) curves between WT and APP/PS1 before and after HCQ application did not show any significant differences (Two-way ANOVA, P=0.99) (FIG. 15G). Comparison of paired pulse ratio (PPR) in WT and APP/PS1 before and after HCQ did not show a significant difference, indicating that HCQ does not affect basal synaptic transmission in either WT or APP/PS1 mice (FIG. 15H) (ANOVA, P=0.09).

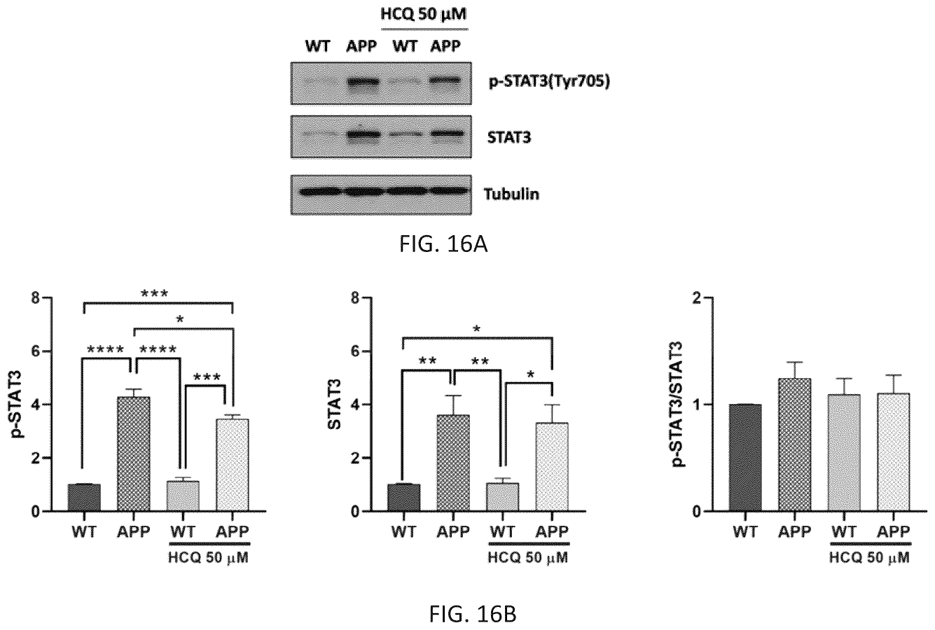

To test if the rescue of impaired late-LTP by HCQ may be through inactivation of STAT3, levels of total STAT3 and phosphorylated STAT3 (p-STAT3; Tyr705) were examined to determine whether they were reduced by HCQ treatment. Specifically, differences in STAT3 and pSTAT3 levels were tested in hippocampal homogenate samples across four groups: WT, APP/PS1, WT+50 μM HCQ, and APP/PS1+50 μM HCQ mice (5-months old). All hippocampal slices were incubated in the interphase chamber and HCQ was applied 30 minutes before and after STET. In each group, hippocampal slices were collected one hour after STET. Levels of total STAT3 and p-STAT3 (Tyr705) were significantly higher in APP/PS1 mice compared to WT mice and were similar between WT and WT+50 μM HCQ mice (FIGS. 16A and 16B). Levels of p-STAT3 (Tyr705) were significantly lower in APP/PS1+50 μM HCQ mice compared to untreated APP/PS1 mice. In addition, levels of total STAT3 were significantly higher in both APP/PS1 and APP/PS1+50 μM HCQ mice compared to WT and WT+50 μM HCQ mice. However, no significant difference in the level of total STAT3 was found between APP/PS1 and APP/PS1+50 μM HCQ mice. This suggests that the rescue of impaired LTP by HCQ in APP/PS1 mice may be partially mediated through inactivation of STAT3 by regulating the expression of plasticity-related proteins which are important for the maintenance of synaptic plasticity.

### Example 8

### HCQ Lowers Risk of AD and Related Dementias

### Methods

The full study protocol for patient-level analysis in Medicare claims was pre-registered on clinicaltrials.gov prior to data analysis (NCT04691505) and contains detailed information on implementation including all codes that were used to identify study variables to allow future replication. The following sections summarize key methodologic details.

Data source: Medicare Fee-For-Service claims data from 2007 through 2017 were used. Medicare Part A (hospitalizations), B (medical services), and D (prescription medications) claims are available for research purposes through the Centers for Medicare and Medicaid Services. A signed data use agreement with the CMS was available and the Brigham and Women's Hospital's Institutional Review Board approved this study.

Study cohort: A new user, active comparator, observational cohort study design comparing methotrexate to hydroxychloroquine was employed. The patients were required to have continuous enrollment in Medicare parts A, B, and D during the baseline period of 365 days before initiation date of methotrexate or hydroxychloroquine, which was defined as the cohort entry date. Patients were required to have ≥1 diagnosis codes of rheumatoid arthritis during the baseline period but no prior use of any disease modifying antirheumatic treatments. Patients with existing diagnosis of Alzheimer's disease and related dementia (ADRD) any time prior to and including cohort entry date were excluded to focus on incident events. Patients with nursing home admission in 365 days prior to and including cohort entry date also were excluded as medication records for short nursing home stays are unavailable in Medicare claims.

Outcome measurement: The endpoint of ADRD was identified based on diagnosis codes, recorded on 1 inpatient claim or 2 outpatient claims, of Alzheimer's disease, vascular dementia, senile, presenile, or unspecified dementia, or dementia in other diseases classified elsewhere. When validated against a structured in-home dementia assessment, Medicare claims-based dementia identification is reported to have a positive predictive value (PPV) in the range of 65% to 78%.

Alternative analytic approaches: To accommodate various uncertainties involved in pharmacoepidemiologic investigations focused on ADRD risk, the following alternative analyses were employed. Analysis 1—‘As-treated’ follow-up approach: In this approach, the follow-up started on the day following the cohort entry date and continued until treatment discontinuation or switch (to comparator treatment), insurance disenrollment, death, or administrative endpoint (December 2017). A 90-day ‘grace period’ after the end of the expected days-supply of the most recently filled prescription was considered to define the treatment discontinuation date to accommodate for suboptimal adherence during treatment periods. Analysis 2—‘As-started’ follow-up approach incorporating a 6-month ‘induction’ period: In this approach, a 6-month induction period was incorporated after the cohort entry date before beginning the follow-up for ADRD and followed patients for a maximum of 3 years regardless of subsequent treatment changes or discontinuation, similar to an intent-to-treat approach in randomized controlled trials. This follow-up approach addresses concerns related to informative censoring if patients discontinue or if physicians de-prescribe the treatments under consideration because of memory problems associated with ADRD, but the diagnosis is not recorded in the electronic healthcare records until after the end of follow-up due to treatment-related censoring. Analysis 3—incorporating a 6-month ‘symptoms to diagnosis’ period: In this approach, an outcome date was assigned that was 6 months before the first recorded ADRD date and excluded last 6 months of follow-up for those who are censored without an event to account for the possibility that ADRD symptoms likely appear some time before a formal diagnosis is recorded in insurance records, which leads to misclassification of ADRD onset. Analysis 4—Alternate outcome definition: In this approach, the outcome was defined using a combination of diagnosis code and ≥1 prescription claim for a symptomatic treatment [donepezil, galantamine, rivastigmine, and memantine] occurring within 6 months of each other with outcome date assigned to second event in the sequence. Use of medication records to identify dementia has been reported to result in >95% PPV in a previous validation study.

Covariates: A large number of covariates were identified, which were measured in the 365-day baseline period preceding the cohort entry date. The following set of variables were included: 1) demographic factors such as age, gender, race, socioeconomic status proxies, 2) risk factors for ADRD identified in previous studies such as diabetes, stroke, and depression, 3) lifestyle factors such as smoking as well as use of preventive services, including screening mammography and vaccinations, to account for healthy-user effects¹³; measures for use of various healthcare services before cohort entry including number of distinct prescriptions filled, number of emergency department visits, hospitalizations, and number of physician office visits to account for patients' general health and contact with the healthcare system to minimize the possibility of differential surveillance; frailty indicators based on composite scoring scheme¹⁵ to address potential confounding by frailty, 4) comorbid conditions and comedications including prior use of pain medications such as steroids and opioids.

Statistical analyses: A propensity-score (PS)-based approach was used to account for measured confounding in this study. The PS were calculated as the predicted probability of initiating the exposure of interest versus the reference drug conditional on baseline covariates using multivariable logistic regression. On average, patients with similar PSs have similar distribution of potential confounders used to estimate the PS. Therefore, analyses conditioned on the PS provide effect estimates that are free from measured confounding. For all our analyses, initiators of each exposure of interest were matched with initiators of the reference exposure based on their PS. Pair matching was conducted using a nearest-neighbor algorithm, which seeks to minimize the distance between propensity scores in each pair of treated and reference patients, and a caliper of 0.025 on the natural scale of the PS was used to ensure similarity between the matched patients. Multiple diagnostics for PS analysis were evaluated including PS distributional overlap before and after matching to ensure comparability of these groups and balance in each individual covariate between two treatment groups using standardized differences.

In the PS matched sample, incidence rates along with 95% confidence intervals for the outcome were estimated for the treatment and reference groups. The competing risk of death could be of concern for the current set of analyses if mortality is frequent among patients included in the cohort and if differences in the risk of mortality between treatment and reference groups are substantial. Therefore, we calculated cumulative incidence using cumulative incidence functions that account for competing risk by death and provided cause-specific hazard ratios from Cox proportional hazards regression model. Pre-specified subgroup analyses were conducted based on age, sex, and baseline cardiovascular disease.

### Results

To test whether exposure to HCQ lowers AD risk in older individuals, insurance claims data from the Medicare program were used to evaluate the comparative risk of incident AD among patients with rheumatoid arthritis who initiated HCQ versus methotrexate (MTX) (clinicaltrials.gov protocol NCT04691505). A new-user active comparator design with propensity-score (PS) based adjustment for confounding, was used to estimate treatment effects in four alternative analyses designed to address various uncertainties associated with claims-based analyses of dementia risk including exposed person-time misclassification, reverse causation, informative censoring, and misclassification of outcome onset as described previously ((Zhao et al., Cell Death Differ2019, 26:1600-1614).

Of the 881,432 patients filling at least one prescription for the drugs of interest (HCQ or MTX) during the study period, 109,124 patients who met all inclusion criteria (54,562 hydroxychloroquine initiators 1:1 PS matched to 54,562 methotrexate initiators) were included. Average age of included patients was 74 years, 76% were females, and 84% were white: exposure groups were comparable on all measured characteristics after PS matching.

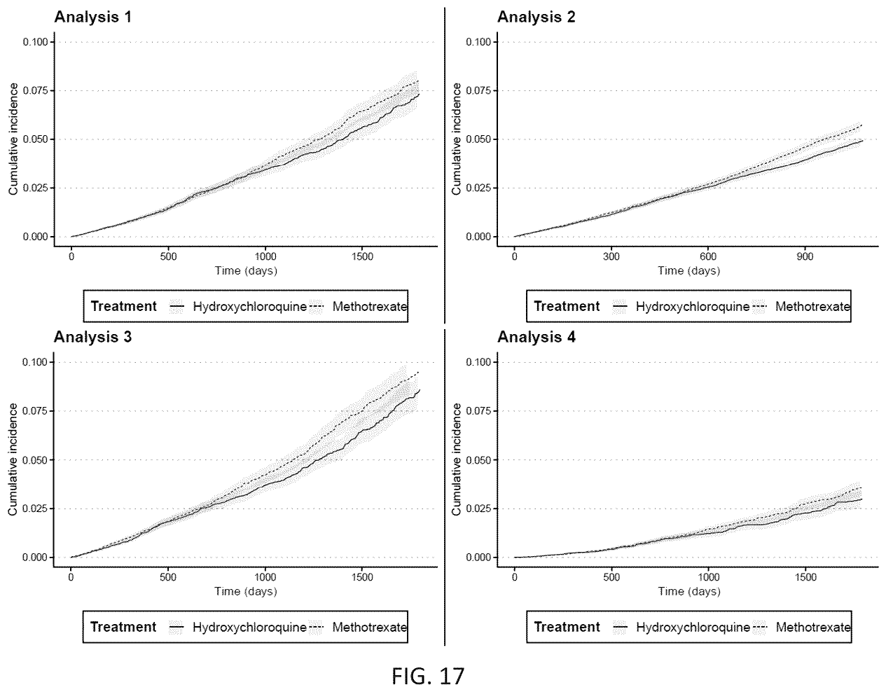

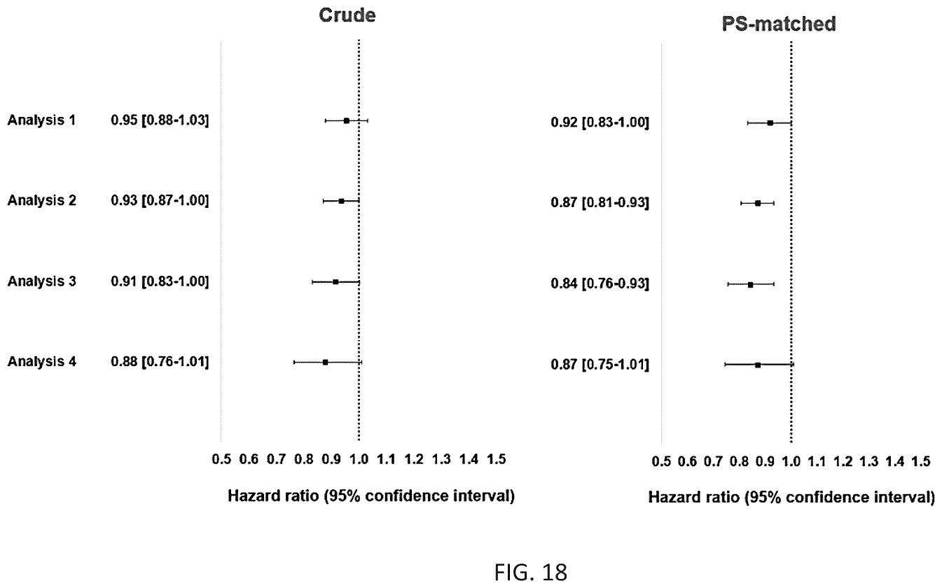

FIG. 17 summarizes cumulative incidence of ADRD among HCQ initiators compared to MTX initiators; results from all four analyses indicate that after approximately 2 years of treatment, individuals on HCQ had lower cumulative incidence of ADRD compared to MTX. FIG. 18 summarizes the crude and PS-matched comparative risk of ADRD in HCQ compared to MTX groups; results indicated that risk of ADRD was consistently lower among HCQ initiators. In Analysis 1 where patient follow-up time was censored at discontinuation of the initial treatment (“as-treated” approach), HCQ initiators had an 8% lower risk of ADRD compared to MTX initiators (HR, 95% CI 0.92, 0.83-1.00). In Analysis 2 where a 6-month induction period was incorporated to eliminate reverse causality and continued follow-up for 3 years regardless of treatment change or discontinuation to minimize the impact of informative censoring in an ‘as started’ follow-up scheme, HCQ was significantly associated with a 13% lower risk (HR, 95% CI 0.87, 0.81-0.93). In Analysis 3 where a 6-month ‘symptom to claims’ period was accommodated to address misclassification of outcome onset, HCQ was significantly associated with a 16% lower risk (HR, 95% CI 0.84 [0.76-0.93). Finally, in Analysis 4, which required symptomatic treatment with cholinesterase inhibitors or Memantine along with diagnosis codes to overcome outcome misclassification due to low sensitivity of the approach relying only on diagnosis codes, effect estimates were largely consistent with other approaches (HR, 95% CI 0.87, 0.75-1.01). No evidence of heterogeneity in treatment effects was observed across subgroups of age, gender, and baseline cardiovascular disease history.

### Discussion of Examples 5-8

The commonly used rheumatoid arthritis drug, HCQ was demonstrated to inactivate STAT3, correct molecular abnormalities underlying AD and lower disease risk in older individuals. Together, these findings suggest that HCQ is a promising disease-modifying AD treatment in at-risk individuals.

Examles 1-3 showed that C188-9, a novel STAT3 inactivator and experimental cancer drug rescued several molecular phenotypes related to AD. These included lowering Aβ₁₋₄₂ secretion in human APP over-expressing neuroglioma cells, reducing tau phosphoryation in neuroblastoma cells overexpressing human mutant tau and reducing neuroinflammation in microglial cells. Together, these findings suggested that inactivation of STAT3 may be a promising therapeutic approach in AD. Considering the recent demonstration by Lyu and colleagues that HCQ inactivates STAT3 in lung adenocarcinoma cells, it was hypothesized that if HCQ similarly inactivated STAT3 in AD-relevant cells, it may ameliorate molecular abnormalities associated with AD. As shown in FIG. 12, it was established that HCQ is an inactivator of STAT3 in neurons, microglia and astrocytes as evidenced by lowering of p-STAT3 levels. The effects of HCQ were tested in phenotypic assays reflecting molecular features of AD pathogenesis to assess its potential as a candidate AD treatment.

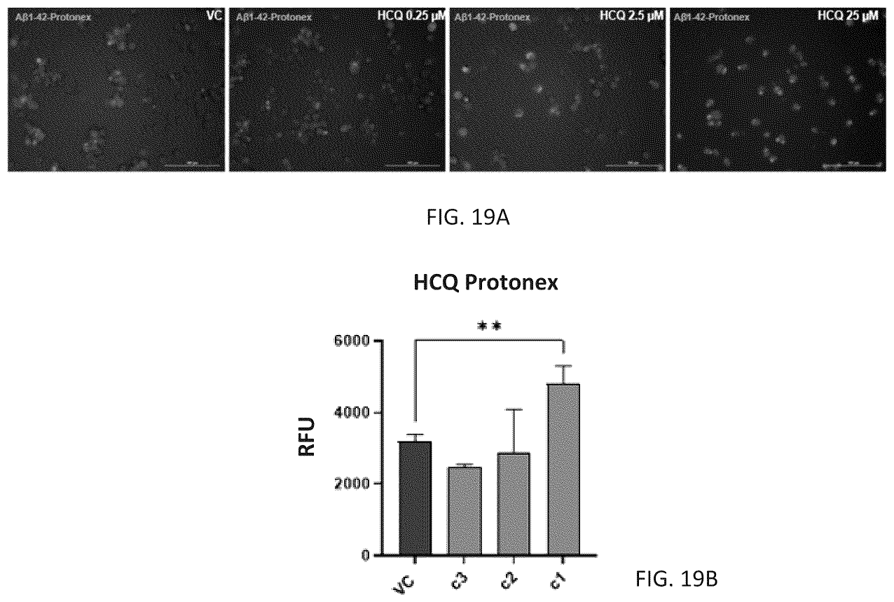

The results suggest that HCQ impacts key features of microglial function relevant to AD pathophysiology that may mediate its therapeutic effects (Lee et al., J Neural Transm (Vienna), 2010, 117(8):949-60; Grubman et al., Nature Comm 2021, 12, article 3015). These include countering neuroinflammation by lowering release of pro-inflammatory cytokines and blocking their downstream effects by inactivating signaling through the cytokine transducer STAT3 (FIGS. 13C, 14C). Impaired phagocytosis and poor clearance of extracellular Aβ by activated microglia is another important pathogenic mechanism in AD. HCQ also appears to enhance the physiological role of microglia in clearing extracellular Aβ₁₋₄₂. As shown in FIG. 14A, pretreatment of BV2 microglial cells with 25 μM HCQ significantly enhanced Aβ₁₋₄₂ clearance as indicated by lower levels in the supernatant and lower ratio of supernatant:lysate Aβ₁₋₄₂ levels. To further assess potential mechanisms underlying this finding, the pH-sensitive fluorescent label, Protonex Green, was used to label Aβ₁₋₄₂ and demonstrated that HCQ pretreatment enhances microglial uptake of Aβ₁₋₄₂ preferentially into acidic cellular compartments such as lysosomes (FIGS. 19A-19B). While HCQ-associated attenuation of neuroinflammation was associated with lower levels of total STAT3, the enhancement of Aβ₁₋₄₂ clearance was associated with reduction in p-STAT3 levels in microglia (FIGS. 19A-19B). Together, these results indicate that HCQ restores critical microglial functions known to be perturbed in AD.

The observation that HCQ reduces tau phosphorylation in neuroblastoma cells overexpressing human mutant tau implicates yet another potential therapeutic mechanism of action in AD. While the precise mechanisms mediating this effect have not been investigated, the finding that lowering of p-tau levels by HCQ is associated with an accompanying reduction in p-STAT3 is consistent with prior studies suggesting that pro-inflammatory cytokine signaling through STAT3 may lead to tau hyperphosphorylation through cyclin-dependent kinase 5 (cdk5). IL-6 induces Alzheimer-type phosphorylation of tau protein by deregulating the cdk5/p35 pathway. Another mechanism that implicates STAT3 in tau phosphorylation is by complement-mediated signaling through C3 and C3a receptor (C3aR1). Complement C3aR1 inactivation attenuates tau pathology and reverses an immune network deregulated in tauopathy models and AD. Given that HCQ impacts the three principal pathogenic molecular pathways in AD i.e., neuroinflammation, Aβ clearance and tau phosphorylation, the next goal was to examine whether HCQ may restore impaired synaptic plasticity which is believed to trigger cognitive impairment in AD. This was evaluated by studying the effects of HCQ on abnormal hippocampal synaptic plasticity characterized by impaired late long-term potentiation (L-LTP) in the APP/PS1 transgenic AD mouse model. L-LTP is a protein-synthesis dependent form of synaptic plasticity important in hippocampal memory formation (Bin Ibrahim et al., FEBS J 2021, doi:10.1111/febs.16065). Previous studies have observed impaired L-LTP in APP/PS1 mice at 3-4 months of age, prior to the accumulation of Aβ plaques (Li et al., PNAS USA 2017, 114(21):5527-5532). This observation allowed testing of pharmacological interventions at the earliest stages of AD, prior to the accumulation of Aβ pathology. The results show that HCQ restores L-LTP in the hippocampus of APP/PS1 mice. At a dose of 25 μM HCQ, restoration of L-LTP was observed that was previously absent in untreated APP/PS1 hippocampal slices, although the magnitude of L-LTP at this dose of HCQ was still lower than that observed in wild type mice. However, at a concentration of 50 μM HCQ, L-LTP in APP/PS1 mice was identical to that in wild type mice, suggesting a complete recovery of impaired hippocampal synaptic plasticity. The western blot analyses suggest that this rescue of L-LTP at the higher concentration of HCQ is associated with inactivation of STAT3 as reflected in lower p-STAT3 levels. The findings are consistent with recent studies showing that pharmacological inactivation of STAT3 rescues impaired learning and memory in the 5xFAD and APP/PS1 mouse models of AD. Inhibition of STAT3 phosphorylation attenuates impairments in learning and memory in 5XFAD mice, an animal model of Alzheimer's disease. Inhibition of STAT3-mediated astrogliosis ameliorates pathology in an Alzheimer's disease model. A potential mechanism proposed to explain these findings is through correction of astrocytic and neuronal hyperactivity, attenuation of neuroinflammation, restoration of Aβ clearance by microglia and a reduction in dystrophic neurites. Previous findings that STAT3 signaling may mediate Aβ-induced neuronal cell death in AD is also a potential mechanism underlying the rescue of hippocampal function by HCQ-induced STAT3 inactivation. Tyk2/STAT3 signaling mediates amyloid-induced neuronal cell death, which has implications in AD.

Building on the cell culture-based phenotypic assays and in vitro studies of synaptic function in APP/PS1 mice that showed promising therapeutic effects of HCQ, an evaluation of whether exposure to HCQ lowers AD risk in humans was performed. A large real-world clinical dataset using Medicare-based claims data was leveraged and a new-user, active comparator observational cohort study was implemented to compare the effect of methotrexate to hydroxychloroquine on incident AD risk in older individuals diagnosed with rheumatoid arthritis. A rigorous study design was implemented that addressed several common pitfalls in pharmacoepidemiological studies that may lead to spurious results in analyses of claims-based data. The findings indicate that exposure to HCQ in older individuals prior to AD diagnosis is associated with 8-16% lowering of incident AD relative to the active comparator, methotrexate. These results suggest that the observations from cell culture-based and transgenic AD model-based experiments indicating therapeutic effects of HCQ in AD may be translated into humans to modify the trajectory of AD. The current state of knowledge of AD progression and potential disease-modifying treatments converges around the consensus that such interventions must be deployed well before symptoms begin and precisely targeted at individuals likely to benefit the most (see, e.g., Cummings et al., Alzheimer's Research and Therapy 2016, 8:39).

Several features make HCQ an attractive candidate for repurposing in AD including its permeability across the blood brain barrier and effective partitioning into the brain. Doses of HCQ of 6.0-6.5 mg/kg/day based on an ideal body weight and typically used in RA patients, yield serum concentrations of 1.4 to 1.5 micromolar (Mackenzie, Am J Med 1983, 75(1A):5-10). Previous studies have shown that brain tissue concentrations of HCQ are several fold higher than in plasma (Kp brain/plasma 21.99±0.11 in macaques, 4-31 in albino rats) (Browning, Hydroxychloroquine and Chloroquine Retinopathy 2014, 35-63; Belkhir et al., Eur J Drug Metab Pharmacokinet 2020, 45(6): 703-13). These studies indicate that at doses of HCQ reflecting those used in RA patients, there is a substantial penetration of HCQ into the brain and accumulation at levels within the range of the doses used in our in vitro studies. Moreover, HCQ and other 4-aminoquinolones have been shown to accumulate in acidic cellular compartments such as lysosomes at millimolar levels (Browning, Hydroxychloroquine and Chloroquine Retinopathy 2014, 35-63). Furthermore, HCQ has a well-established safety profile with serious side effects of retinopathy and cardiac toxicity being relatively rare (Nirk et al., EMBO Mol Med 2020, 12:e12476).

In summary, it was established that the commonly used RA drug, HCQ inactivates STAT3 and targets multiple pathogenic mechanisms in AD including neuroinflammation, Aβ clearance, tau phosphorylation and synaptic dysfunction. HCQ also lowers AD risk in older individuals. The results provide compelling evidence that this safe and inexpensive drug is a promising disease-modifying treatment for AD.

### Example 9

### HCQ Reduces Neuroinflammation in AD Mouse Model

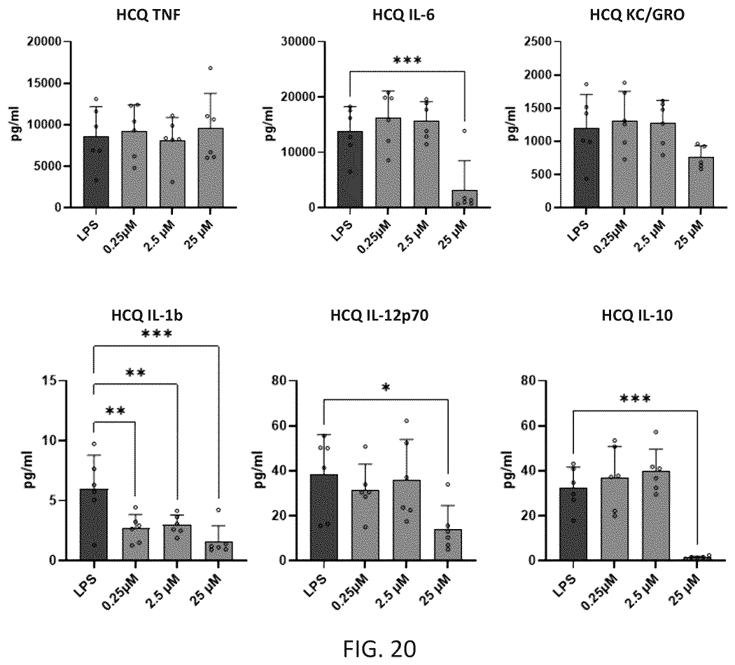

HCQ was confirmed to exhibit anti-inflammatory effects in a model of neuroinflammation using microglial cells from the brains of a transgenic AD mouse model. The results showed that, similar to observations in BV2 microglial cells, HCQ lowered the release of several cytokines, including IL6, IL1-b, IL-10, and IL-12p70. The results are shown in FIG. 20.

### Methods

MACS microglia isolation and cultivation: Adult microglia were isolated from 9 months old 5xFAD mice via magnetic cell sorting (MACS). The mice were terminally anesthetized by i.p. injection of Pentobarbital (600 mg/kg, dosing 10 μL/g body weight) and brains were transcardially perfused with DPBS. Brains were removed, the brainstem discarded and the remaining brain minced for cell dissociation. Cell dissociation was performed using Miltenyi Adult Brain Dissociation Kit (Miltenyi, 130-107-677). MACS cell separation was performed using CD11b (Microglia) MicroBeads, mouse (Miltenyi, 130-093-634) and MS columns on OctoMACS™ cell separator (Miltenyi). Isolated microglia were seeded onto 0.01% PLL coated plates at a density of 10.000 cells per well in 384 well plate in DMEM containing 10% FBS,1% penicillin/streptomycin, and 2 mM L-glutamine.

LPS-induced neuroinflammation on MACS isolated 5xFAD microglia: On DIV7 HCQ at 0.25 μM, 2.5 μM and 25 μM was applied 1 hour before LPS stimulation (Sigma-Aldrich; L6529; 1 mg/ml stock in ddH₂O, final concentration in well: 50 ng/ml (dilutions in medium)). Cells treated with vehicle, cells treated with LPS alone, as well as cells treated with LPS plus reference item (dexamethasone 10 μM, Sigma D4902) served as controls. After 24 h of stimulation, cell supernatants were collected for the cytokine measurement (V-PLEX Proinflammatory Panel 1 Mouse Kit, K15048D, Mesoscale).

### Example 10

### Treatment with Tauroursodeoxycholic Acid (TUDCA) or HCQ+TUDCA

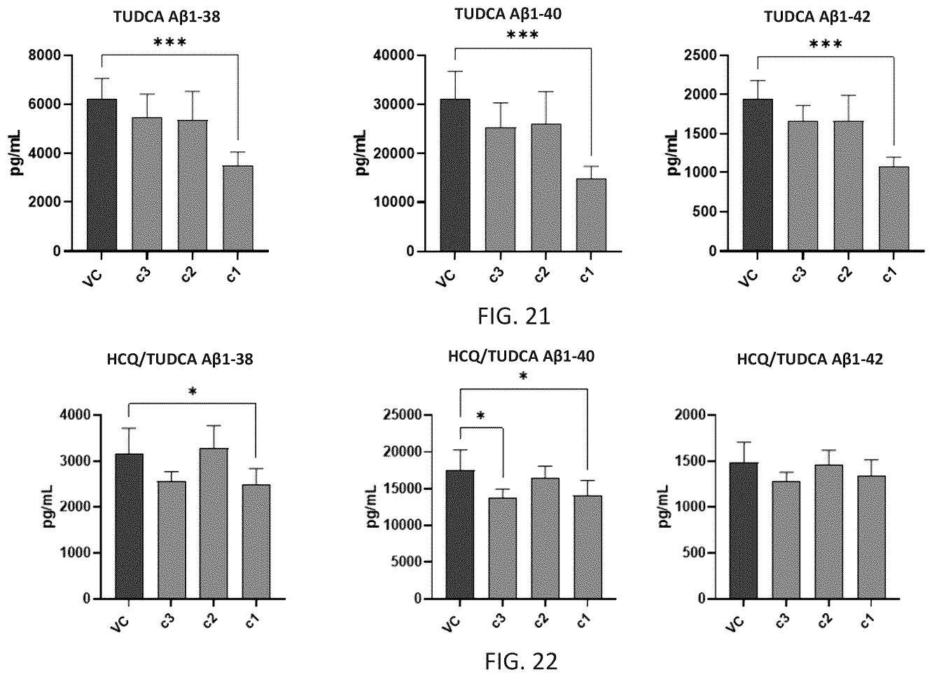

TUDCA was evaluated alone and in combination with HCQ. Prior work has shown that bile acid metabolism may be a plausible drug target in AD and related dementias (Varma et al., PLOS Medicine, May 27, 2021, 18(5):e1003615). The cell culture-based results in FIG. 21 show that TUDCA lowers the secretion of Aβ1-38, Aβ1-40, and Aβ1-42, whereas HCQ increased microglial clearance of exogenous Aβ1-42 (FIG. 13A). FIG. 22 shows the effect of HCQ in combination with TUDCA.

### Methods

Cultivation and treatment of H4-hAPP cells: H4-hAPP cells were thawed and cultivated in Opti-MEM supplemented with 10% FCS, 1% penicillin/streptomycin 200 μg/ml Hygromycin B and 2.5 μg/ml Blasticidin S (=culture medium). H4-hAPP cells were seeded into 96 well plates (2×10⁴ cells per well). On the next day, cells in 96 well plates were treated with TUDCA (100 μM (C1), 10 μM (C2), or 1.0 μM (C3)), R.I. (DAPT 400 nM) or vehicle. Alternatively, cells in 96 well plates were treated with 25 μM HCQ/0.1 μM TUDCA (C1), 2.5 μM HCQ/100 μM TUDCA (C2), and 2.5 μM HCQ/10 μM TUDCA (C3). 24 h later, supernatants were collected for further Aβ measurements by MSD. The experiment was performed in n=6 technical replicates for all groups.

Aβ38, 40, 42 measurement: Supernatants were diluted 1:10 and analyzed for human Aβ38, 40, and 42 with MSD® 96-well MULTI-SPOT® 6E10 Abeta Triplex Assay (Mesoscale Discovery, Rockville, MD). The immune assay was carried out according to the manual and plates are read on the MESO QuickPlex SQ 120 multiplexing instrument. Analyte levels were evaluated according to adequate Aβ peptide standards (MSD) as pg per mL.

Statistics: Basic statistical analysis was performed. Data are presented as mean±standard error of mean (SD) and group differences are evaluated for each Test items separately by one-way ANOVA in GraphPad Prism 9. In FIGS. 21 and 22, data are presented as bar graphs with group mean+SD (n=6 per group). VC=vehicle control 0.1% DMSO. In FIG. 21, C1-C3 are 100 μM, 10 μM, and 1.0 μM TUDCA, respectively. In FIG. 22, C1-C3 are 25 μM HCQ/0.1 μM TUDCA, 2.5 μM HCQ/100 μM TUDCA, and 2.5 μM HCQ/10 μM TUDCA, respectively. One-way ANOVA followed by Dunnetts multiple comparison test versus VC: * p<0.05; **p<0.01 ;***p<0.001.

### Example 11

### TUDCA and DIM Evaluation in AD-Related Cell-Based Assays

TUDCA and DIM were investigated to determine whether they could rescue molecular phenotypes relevant to AD, including tau phosphorylation, Aβ1-42 clearance, Aβ secretion, Aβ toxicity, lipopolysaccharide (LPS)-induced neuroinflammation, cell death due to trophic factor withdrawal, and neurite outgrowth and neurogenesis (Varma, PloS one 2020, 15(32271844):e02314446; Desai et al., Brain Communications 2022, 4(5):10). TUDCA and DIM were selected based on brain penetration/permeability across the blood brain barrier, close network proximity of the drug target genes to AD risk genes through short path lengths connected by single mediator genes, and prior evidence for roles in molecular mechanisms relevant to neurodegeneration. In all assays, cells treated with drugs were compared to either a vehicle control (VC) or lesion control.

### Methods

Cell-based assays: For each combination of drugs and assay, the posterior probability P(Hᵢ|data) of protective, neutral, and adverse drug effect (formal hypotheses H₁, H₀, H₂, respectively) were evaluated. Dose-response relationships (x-y data) were modeled with a Bayesian nonlinear regression model:

$$y ∼ N ( μ , {σ}_{μ} )$$

$${σ}_{μ} = σ μ$$

$$μ = \vert {y}_{1} + \frac{{y}_{0} - {y}_{1}}{( 1 + exp ( k * x - {EC}_{50} ) ) )} \vert$$

The parameter y₀, y₁ was interpreted as expected bioactivity at zero and saturating drug concentration, and fold change was defined as FC_{y}=y₁/y₀. A gamma prior density was chosen for the fold change as:

$${FC}_{y} ∼ Γ ( α = 5 , β = 5 )$$

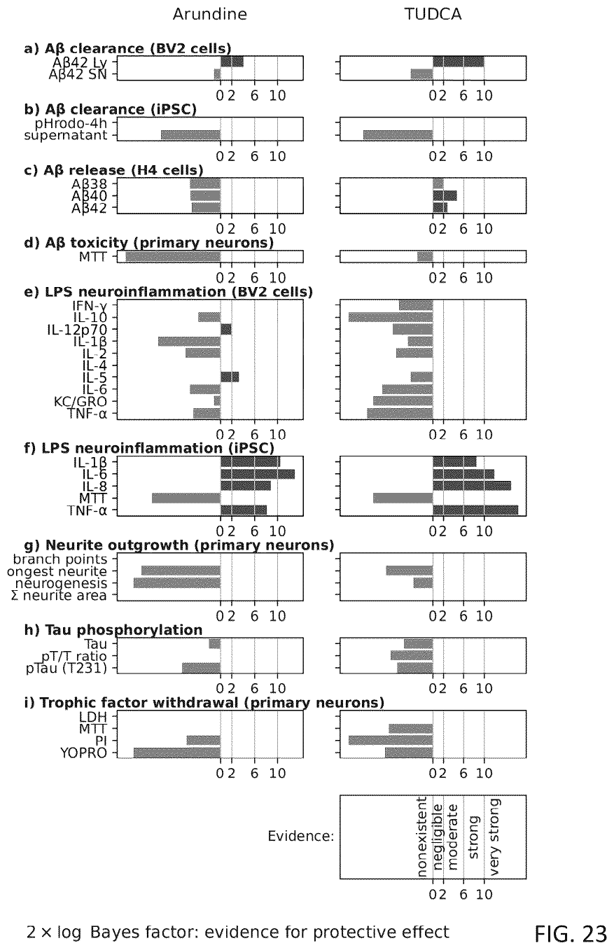

and Bayesian estimation of the posterior density of the fold change was performed using default settings of the Markov chain Monte Carlo samples implemented by the PyMC python library. For efficient sample, each bioactivity data set was standardized such that 10 units corresponded to one standard deviation. Diagnostic criteria (posterior sample of fitted curves, effective sample sizes, values of the \hat{r} statistic, and Markov chain standard errors) were inspected for the fit and three poorly fitted data sets out of a total of 91 data sets were excluded. The foregoing analysis was used to generate data presented in FIG. 23 (infra).

Aβ Clearance: 20,000 BV2 cells per well (uncoated 96-well plates were plated out. After changing cells to treatment medium, drug compounds were administered 1 hour before Aβ stimulation (final concentration in well 200 ng/ml; dilutions in medium). Cells treated with vehicle and cells treated with Aβ alone served as controls. After 3 hours of Aβ stimulation, cell supernatants were collected for the Aβ measurement and cells were carefully washed twice with PBS and thereafter lysed in 35 μl of cell lysis buffer (50 mM tris-HCl, pH 7.4, 150 mM NaCl, 5 mM EDTA, and 1% SDS) supplemented with protease inhibitors. Supernatants and cell lysates were analyzed for human Aβ42 with the MSD V-PLEX Human Aβ42 Peptide (6E10) Kit (K151LBE, Mesoscale Discovery). The immune assay was carried out according to the manual, and plates were read on MESO QuickPlex SQ 120 (Meso Scale Diagnostics, Rockville, MD).

Aβ Secretion: H4-hAPP cells were cultivated in Opti-MEM supplemented with 10% FCS, 1% penicillin/streptomycin, hygromycin B (200 μg/ml), and blasticidin S (2.5 μg/ml). H4-hAPP cells were seeded into 96-well plates (2×104 cells per well). On the next day, cells in 96-well plates were treated with compounds, reference item [400 nM N-[N-(3,5-difluorophenacetyl-1-alanyl)]-S-phenylglycine t-butyl ester (DAPT)], or vehicle. Twenty-four hours later, supernatants were collected for further Aβ measurements by MSD [V-PLEX Aβ Peptide Panel 1 (6E10) Kit, K15200E, Mesoscale Discovery].

Tau Phosphorylation: SH-SY5Y-hTau441(V337M/R406W) cells were maintained in culture medium [DMEM medium, 10% FCS, 1% nonessential amino acids (NEAA), 1% l-glutamine, gentamycin (100 μg/ml), and geneticin G-418 (300 μg/ml)] and differentiated with 10 μM retinoic acid for 5 days changing medium every 2 to 3 days. Before the treatment, cells were seeded onto 24-well plates at a cell density of 2×105 cells per well on day one of in vitro culture (DIV1). Drug compounds were applied on DIV2. After 24 hours of incubation (DIV3), cells on 24-well plates were harvested in 60 μL of RIPA buffer [50 mM tris (pH 7.4), 1% NP-40, 0.25% Na-deoxycholate, 150 mM NaCl, 1 mM EDTA supplemented with freshly added 1 μM NaF, 0.2 mM Na-orthovanadate, 80 μM glycerophosphate, protease (Calbiochem, Millipore Sigma, Burlington, MA), and phosphatase (Sigma-Aldrich, St. Louis, MO) inhibitor cocktail]. Protein concentration was determined by BCA assay (Pierce, Thermo Fisher Scientific, Waltham, MA), and samples were adjusted to a uniform total protein concentration. Total tau and phosphorylated tau were determined by immunosorbent assay from Mesoscale Discovery [Phospho(Thr231)/Total Tau Kit K15121D, Mesoscale Discovery, Meso Scale Diagnostics].

LPS-induced Neuroinflammation: The murine microglial cell line BV2 was cultivated in Dulbecco's modified Eagle's medium (DMEM) medium supplemented with 10% fetal calf serum (FCS), 1% penicillin/streptomycin, and 2 mM 1-glutamine (culture medium). For LPS stimulation assay, 5000 BV2 cells per well (uncoated 96-well plates) were plated out and the medium was changed to treatment medium (DMEM, 5% FCS, and 2 mM l-glutamine). After changing cells to treatment medium, drug compounds were administered 1 hour before LPS stimulation [Sigma-Aldrich; L6529; 1 mg/mL stock in ddH2O; final concentration in well, 100 ng/ml (dilutions in medium)]. Cells treated with vehicle, cells treated with LPS alone, and cells treated with LPS plus reference item (dexamethasone, 10 μM; Sigma-Aldrich, D4902) served as controls. After 24 hours of stimulation, cell supernatants were collected for the cytokine measurement (V-PLEX Proinflammatory Panel 1 Mouse Kit, K15048D, Mesoscale) and cells were subjected to 3-(4,5-dimethylthiazol-2-yl)-2,5-diphenyltetrazolium bromide (MTT) assay.

Neurite Outgrowth and Neurogenesis: Primary hippocampal neurons were prepared from E18.5 timed pregnant C57BL/6JRccHsd mice as previously described. Cells were seeded in poly-D-lysine pre-coated 96-well plates at a density of 2.6×104 cells/well in medium (Neurobasal, 2% B-27, 0.5 mM glutamine, 25 μM glutamate, 1% Penicillin-Streptomycin). Directly on DIV1, the drug of interest or VC was applied. On DIV2, 10 μM bromodeuxyuridine (BrdU; B5002 Sigma Aldrich) was added and cells were fixed after an additional 24 h. Cells were permeabilized with 0.1% Triton-X and incubated with primary Beta Tubulin Isotype III (T8660, Sigma Aldrich) and BrdU antibodies (MAS25° C., AHrlan-Sera Lab) overnight at 4° C. Afterwards, cells were washed two times with PBS and incubated with fluorescently labelled secondary antibodies and DAPI for 1.5 h at room temperature (RT) in the dark. Cells were rinsed three times with PBS and imaged with the Cytation 5 Multimode reader (BioTek, Winooski, VT) at 10× magnification (six images per well). BrdU-positive cells were counted as a marker of neurogenesis and Beta Tubulin Isotype III signal was used for macro-based quantification of neurite outgrowth.

Trophic Factor Withdrawal: Primary cortical neurons from embryonic day 18 (E18) C57Bl/6 mice were prepared as previously described. On the day of preparation (DIV1), cortical neurons were seeded on poly-d-lysine pre-coated 96-well plates at a density of 3×104 cells per well. Every 4 to 6 days, a half medium exchange using full medium (Neurobasal, 2% B-27, 0.5 mM glutamine, and 1% penicillin-streptomycin) was carried out. On DIV8, a full medium exchange to B-27 free medium (Neurobasal, 0.5 mM glutamine, and 1% penicillin-streptomycin) was performed and drug compounds were applied thereafter. The experiment was carried out with six technical replicates per condition, and vehicle-treated cells served as control. After 28 hours on B-27-free medium, cells were subjected to YO-PRO/propidium iodide (PI) and MTT as well as lactate dehydrogenase (LDH) assay.

### Results

TUDCA and DIM were evaluated experimentally in 33 AD-related cell-based assays from 9 experiments. Both TUDCA and DIM were found to be protective with high probability in several assays. However, each drug was also found to have an adverse effect in several other assays. A high-level summary of the results is presented in FIG. 23. Both TUDCA and DIM exhibited strongly protective effects in Aβ clearance (BV2 cells) and LPS induced neuroinflammation (iPSCs). Moreover, TUDCA also proved to be protective in Aβ release (H4 cells), whereas DIM was protective in LPS induced neuroinflammation (BV2 cells).

### Discussion

Network pharmacology analyses were combined with cell-based experimental validation to discover novel drug repurposing opportunities in AD. Using cell culture-based phenotypic assays, both DIM and the TUDCA were found to exhibit protective effects given several molecular perturbations relevant to AD pathogenesis.

### Example 12

### Effects of HCQ, TUDCA, and DIM on Plasma NfL Levels in Mice

### Methods

HCQ nano (poly(L-lysine succinylated)-HCQ, or PLS-HCQ) preparation: 2.5 mL of saline were added to each vial of lyophilized material on each dose day and vortexed briefly to mix. Fresh vials were prepared daily. Solution was pale yellow in color and was protecedt from light. Remaining material was discardedafter animals were successfully dosed each day. Vehicle 100 mg/kg, i.p. injection 10 μL/g b.w.

TUDCA preparation: Dissolved in saline; treatment dosage 500 mg/kg; i.p. injection 10 μL/g b.w.

Animal management: Animals were housed in individual ventilated cages on standardized rodent bedding supplied by Rettenmaier. Each cage contained a maximum of five mice. The temperature in the keeping room was maintained between 20 to 24° C. and the relative humidity was maintained between 45 to 65%. Animals were housed under a constant light-cycle (12 hours light/dark). Dried, pelleted standard rodent chow (Altromin) as well as normal tap water was available to the animals ad libitum. Only animals in apparently good health condition were included to the study. Randomization of group allocation was done per cage. If possible, animals were assigned to different starting groups (cohorts) comprising animals of all treatment groups. The number of animals in a starting group was limited to ensure same age and uniform handling. Age at treatment start 7.5 months±0.5 months.

Study Design: 112 transgenic 5xFAD mice were randomly allocated to treatment groups, each consisting of 16 animals. All animals were treated with test compound or vehicle for the whole study period by i.p. injection (Table 1). Group A was dosed daily for the whole treatment period. All other groups were dosed daily in the first 4 weeks, three times per week in weeks 5 to 8, and once weekly until the end of the study. 5xFAD (Familiar Alzheimer Disease) mice bear five mutations, three in the amyloid precursor protein (APP695) gene [APP K670N/M671L (Swedish), I716V (Florida), V717I (London)] as well as two mutations in the presenilin 1 gene [PS1 M146L, L286V] (Oakley et al., 2006). The expression of the 5xFAD transgene is driven by the neuron specific Thy1 promoter. The five mutations cause an early onset of the cognitive decline and increasing Aβ 1-40 and 1-42 levels in the brain and cerebrospinal fluids, over age. Histological analysis revealed plaque load and beta sheet formation accompanied with neuroinflammation. Thus, the 5xFAD mouse mimics the most crucial phenotypic symptoms of amyloidogenic neurodegeneration, neuroinflammation as well as leaming and memory deficits and is a suitable model for Alzheimer's disease to study effects of drugs on biochemical, histological and behavioral hallmarks.

TABLE 1
Treatment Groups

| Group | n | Genotype | Test Item | Sex | Route | Frequency | Duration | Dose |
|---|---|---|---|---|---|---|---|---|
| A | 16 | tg | HCQ | mixed | i.p. | week 1-4: daily | 14 weeks | 100 mg/kg |
| | | | | | | week 5-8: 3 times per | | |
| | | | | | | week, week 9-end: once | | |
| | | | | | | per week | | |
| B | 16 | tg | HCQ nano | mixed | i.p. | week 1-4: daily | 14 weeks | 100 mg/kg |
| | | | | | | week 5-8: 3 times per | | |
| | | | | | | week, week 9-end: once | | |
| | | | | | | per week | | |
| C | 16 | tg | Vehicle | mixed | i.p. | week 1-4: daily | 14 weeks | — |
| | | | [Saline] | | | week 5-8: 3 times per | | |
| | | | | | | week, week 9-end: once | | |
| | | | | | | per week | | |
| D | 16 | wt | TUDCA | male | i.p. | 2 times per week | 14 weeks | 500 mg/kg |

tg = transgenic;
wt = wild type

In-vivo blood samples were collected by mandibular sampling from each animal, at baseline (before treatment start), and after 4, 8 and 12 weeks of treatment. Maximum allowed blood volume was sampled into K₂EDTA (potassium ethylenediaminetetraacetic acid) tubes. The tube was inverted thoroughly to facilitate homogeneous distribution of the EDTA and prevent clotting. Blood plasma was isolated by centrifugation (3000×g for 10 minutes at room temperature (22° C.)) and plasma aliquots were transferred to 1.5 mL tubes, frozen on dry ice and stored at −80° C.

The NF-light® (Neurofilament-light) ELISA 10-7001 CE from UmanDiagnostics was used for analysis. The following samples were analyzed: terminal CSF, terminal blood plasma as well as all 4 timepoints from the in vivo bleedings from 6 animals per groups (3 groups; 18 animals, 108 samples in total). Samples were diluted (1:3 for plasma, 1:35 for CSF) in assay buffer and analyzed according to the manufacturers protocol. CSF and in vivo samples are analyzed in single replicates only, due to limited sample volume. In brief, after dilution, 100 μl of sample are added to the pre-coated wells and incubated for 1.5 hours at RT with gentle agitation (800 rpm). Wells were washed three times with assay wash buffer and 100 μL of the tracer antibody were added. After 45 min incubation (RT, 800 rpm) wells were again washed three times. Thereafter 100 μL of conjugate were added and incubated for 30 min (RT, 800 rpm). After 3× washing 100 μl of TMB substrate were added to each well and incubated for 15 min at RT. 50 μL stop reagent were added and after short gentle agitation, the plate was read at 450 nm (reference 620-650 nm) on the Cytation® 5 multimode reader (Biotek, Agilent Technologies, Inc., Santa Clara, CA). Data were evaluated in comparison to calibration curves provided in the kit and are expressed as pg/ml plasma.

### Results

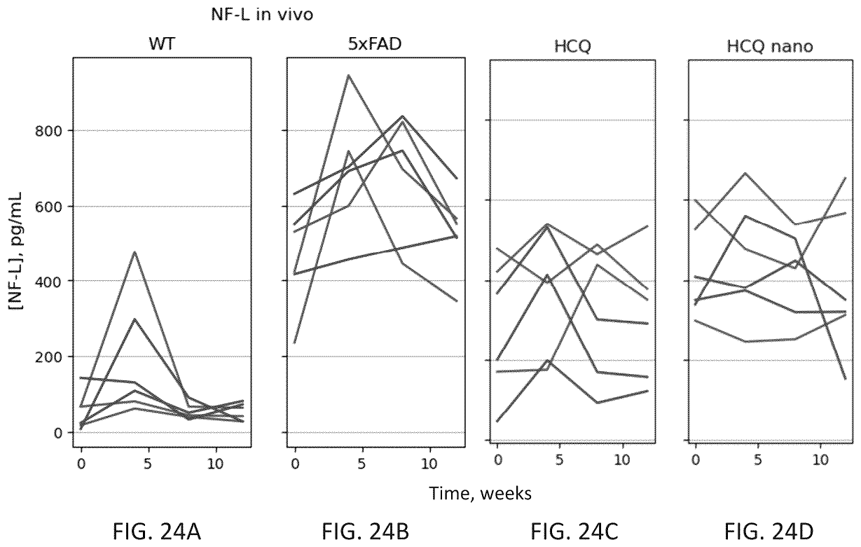

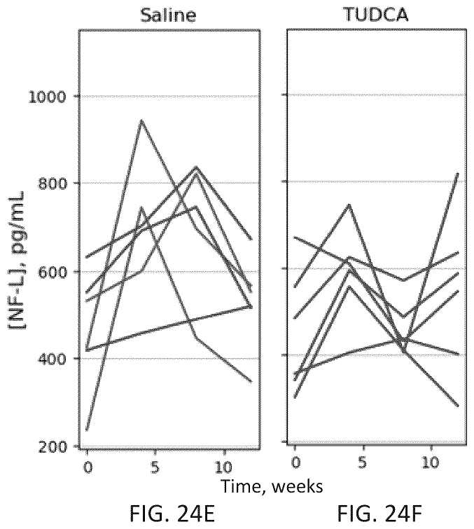

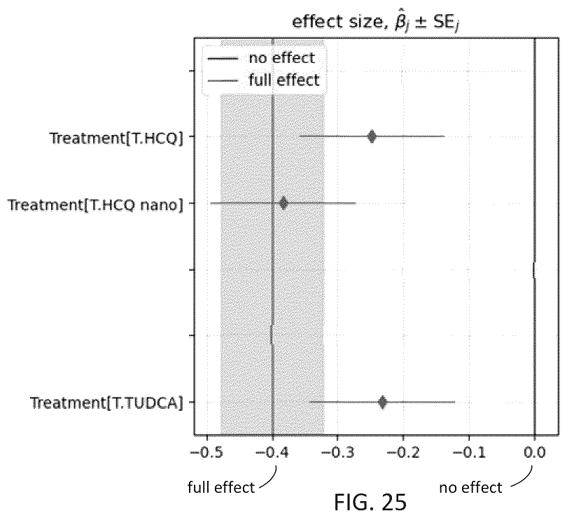

Analyses were performed to test whether candidate AD drugs rescue the peak in plasma NfL levels observed in drug-treated Tg 5xFAD versus saline treated animals. These analyses accounted for the individual variability in NfL levels between animals. NfL was measured in blood plasma drawn at 0, 4, 8, and 12 weeks after injection. Both wild type (FIG. 24A) and 5xFAD mice (FIG. 24B), injected with saline, showed a pronounced NfL peak between 4 and 8 weeks, relative to the baseline NfL level at week 0 (FIG. 24A). FIGS. 24C-24E show the effects on NfL concentration in 5xFAD mice treated with HCQ (FIG. 24C), HCQ nano (FIG. 24D), saline (FIG. 24E) and TUDCA (FIG. 24F); HCQ doses were 100 mg/kg and the TUDCA dose was 500 mg/kg. To gain quantitative support for this finding, the log-transformed baseline-to-peak fold-change of NfL on the indicator variable of drug treatment was regressed and the corresponding regression coefficient beta_j was estimated (FIG. 25). HCQ nano fully prevented the peak in plasma NfL levels compared to saline-treated transgenic mice (p<0.001). TUDCA and HCQ partially lowered the peak plasma NfL levels compared to saline-treated transgenic mice (p<0.05).

### Example 13

### Effects of HCQ, DIM and TUDCA on Synaptic Plasticity in APP/PS1 Mice

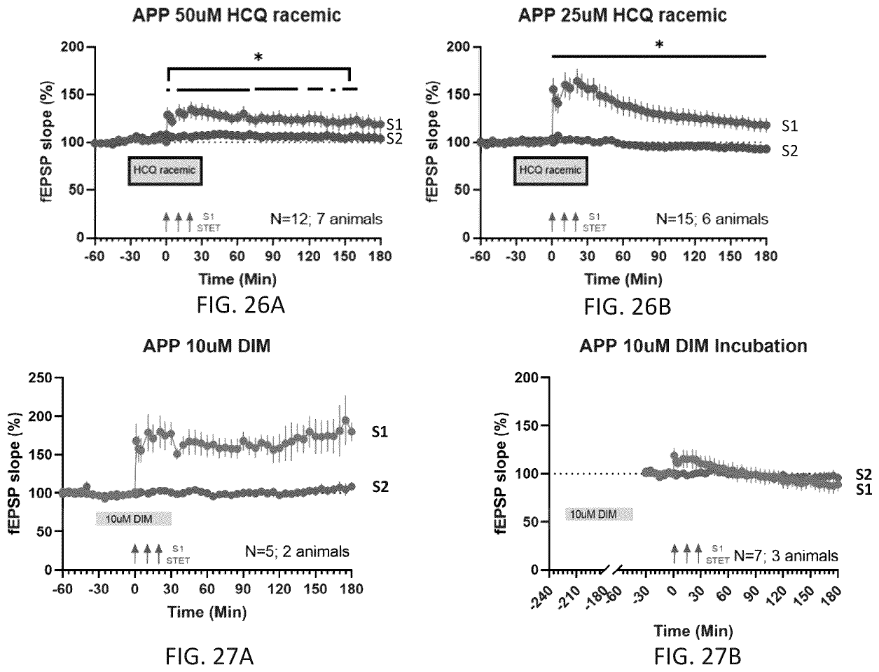

To investigate the effect of HCQ racemic free base on APP/PS1 hippocampal synaptic plasticity, electrophysiological experiments were performed utilizing the mixture at both 25 μM and 50 μM concentrations. Hippocampal slices from 4-5 month old APP/PS1 mice were perfused with 50 μM HCQ racemic free base (FIG. 26A) or 25 μM HCQ racemic free base (FIG. 26B) for 1 hour (−30 min to +30 min) after 30 min stable baseline recordings were obtained. Thirty minutes after the introduction of HCQ, long-term potentiation (LTP) was induced in synaptic input 1 (S1) via a strong tetanizing (STET—8% sucrose, 5% Triton® X-100, 50 mM EDTA, 50 mM Tris HCl—G-Biosciences, St. Louis, MO) protocol, consisting of 3 tetanizing trains (arrows). 25 μM HCQ resulted in significant potentiation (155.545±12.309%) that sustained till the end of the experiment (118.003±7.753%), with S1 slope values being significantly different from unstimulated S2 pathway throughout the experiment (FIG. 37B. However, 50 μM HCQ resulted in slight potentiation (128.809±8.116%) that was no longer statistically relevant by the end of the experiment (119.117±8.167%). The S1 slope values were mostly significantly different up until the 160^{th} minute. The independent S2 pathways in both conditions were stable throughout the experiments. * represents p<0.05, t-test at each time point between S1 and S2.

Effects of the HCQ racemic free base differed from that of the previously tested HCQ sulfate. 25 μM HCQ racemic free base resulted in partial rescue of late-LTP in APP/PS1 mice, similar the HCQ sulfate. However, 50 μM HCQ racemic free base had a diminished initial potentiation which did not last, as compared to 50 μM HCQ sulfate, which had complete rescue. Concentration-dependent effects may have manifested in the 50 μM trials. A preliminary (n=1) test using age-matched WT mice supported this hypothesis with the observation that late LTP was partially impaired in these mice with 50 μM HCQ racemic free base.

To investigate efficacy of DIM in rescuing impaired LTP in APP/PS1 mice at a threshold concentration, field electrophysiological experiments were performed utilizing 10 μM DIM. DIM has an acute rescue effect on LTP in APP/PS1 slices. FIG. 27A shows that the application of 10 μM DIM rescues LTP in APP/PS1 slices. FIG. 27B shows that a 3-hour pre-incubation with 10 μM DIM no longer results in rescue of late LTP in APP/PS1 slices.

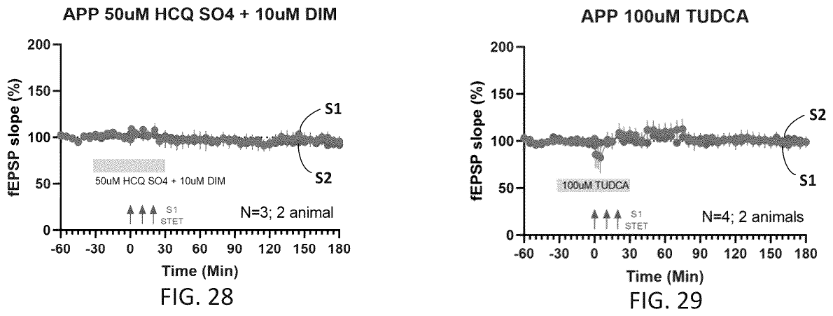

Application of a mixture of 40 μM HCQ sulfate and 10 μM DIM blocked expression of early-LTP upon delivery of STET in APP/PS1 slices (FIG. 28). TUDCA was also found to have a deleterious effect on expression of LTP. STET no longer induced LTP in APP/PS1 mice when applied under the influence of 100 μM TUDCA (FIG. 29).

### Example 14

### Effects of HCQ, DIM, and TUDCA on Aβ1-40 and 1-42 in 5xFAD Mice

Transgenic 5xFAD mice were randomly allocated to treatment groups. The expression of the 5xFAD transgene is driven by the neuron specific Thyl promoter. The five mutations cause an early onset of the cognitive decline and increasing Aβ 1-40 and 1-42 levels in the brain and cerebrospinal fluids, over age. Histological analysis revealed plaque load and beta sheet formation accompanied with neuroinflammation. Thus, the 5xFAD mouse mimics the most crucial phenotypic symptoms of amyloidogenic neurodegeneration, neuroinflammation as well as learning and memory deficits and is a suitable model for Alzheimer's disease to study effects of drugs on biochemical, histological and behavioral hallmarks. All animals were treated with test compound or vehicle for the whole study period by i.p. injection and then euthanized. Male mice administered TUDCA+HCQ were dosed with TUDCA on M/F and HCQ all other days for weeks 1-4, then TUDCA on M/F and HCQ on T/W/Th for weeks 5-8, and then TUDCA on M/F and HCQ on W for weeks 9-14. Animals or mixed sex in other groups were administered the pertinent compounds daily for the 14-week period.

The frozen cortex samples from animals of each group were homogenized in lysis buffer (THB; 250 mM Sucrose, 1 mM EDTA, 1 mM EGTA, 20 mM Tris pH 7.4) including 1× protease inhibitor (Calbiochem). The tissue was homogenized with a beadmill (UPHO, Geneye) at 55 Hz for 50 sec. Aliquots were stored at 80° C. until further use. For extraction of non-plaque associated proteins, 1 aliquot of THB homogenate was mixed with 1 part diethylamine (DEA) solution (0.4% DEA, 100mM NaCl). The mixture was centrifuged for 120 min at 20,000×g, 4° C. The supernatant was neutralized with 1/10 of the volume 0.5 M Tris-HCl, pH 6.8 and vortexed briefly. Aliquots were stored at −80° C. as DEA fraction (soluble fraction). For extraction of deposited proteins, a second aliquot of THB homogenate was mixed with 2.2 parts cold formic acid (FA), sonicated for 30 sec on ice and centrifuged for 120 min at 20,000×g, 4° C. The supernatant was mixed with 19 parts FA Neutralization Solution (1M Tris, 0.5 M Na2HPO4, 0.05% NaN3). Aliquots were stored at 80° C. as FA fraction (insoluble fraction). For measurement of inflammation markers, the third aliquot of homogenate was substituted with Triton® X-100 (Dow Chemical Company, Midland, MI) so that the final concentration as 1% in the homogenate. After vortexing and 10 min incubation on ice, the homogenates were cleared from cell debris by centrifugation at 20,800×g at 4° C. for 10 minutes in a tabletop centrifuge and the supernatants were collected for the measurement of cytokines and stored at −80° C. until further use as Triton® fraction.

Aβ 1-40 and Aβ 1-42 levels in DEA and FA fraction: Aβ 1-40 and Aβ 1-42 were measured in duplicates in the fractions described above (DEA and FA fractions) using Aβ1-42 with MSD® Human (6E10) V-plex kit (K151LBE-2, Mesoscale Discovery) and Aβ1-40 with MSD® Human (6E10) V-plex kit (K150SKE-2, Mesoscale Discovery) according to the instructions of the manufacturer. Plates were read on Quickplex SQ 120 sector imager (Mesoscale Discovery). Aβ levels in study samples were evaluated in comparison to calibration curves provided in the kit and are expressed as pg per mg brain wet weight.

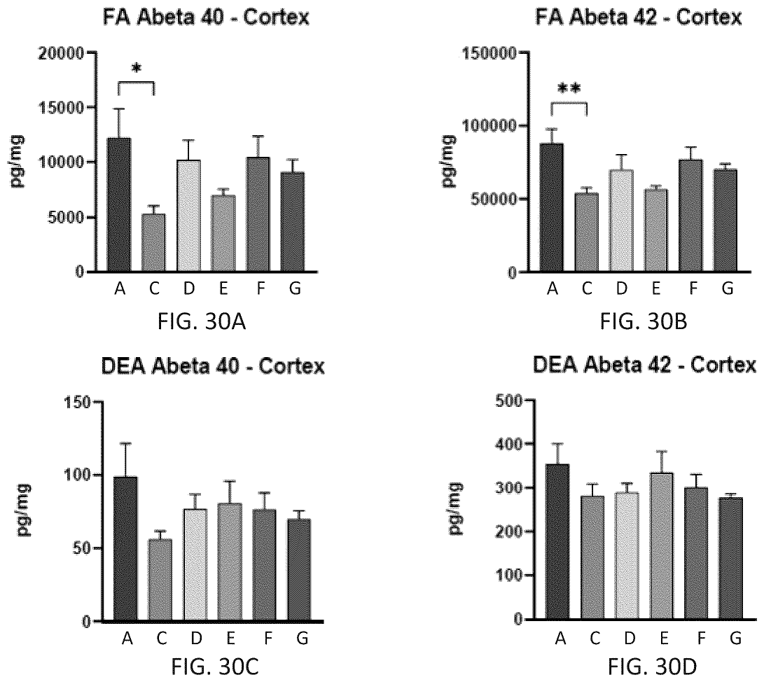

Human Aβ 1-40 and Aβ 1-42 levels were measured in the FA and DEA soluble fractions of the cortex of 5xFAD mice after receiving different treatments (FIGS. 30A-30D). Group A—transgenic mice, saline; Group C—tg mice, TUDCA 500 mg/kg+HCQ 100 mg/kg; Group D—tg mice, DIM 50 mg/kg; Group E—tg mice, DIM 100 mg/kg; Group F—tg mice, DIM 200 mg/kg; Group G—tc mice, vehicle (10% DMSO, 10% pEG300, 10% Tween® 80 surfactant (Sigma-Aldrich, St. Louis, MO), 70% H₂O). TUDCA+HCQ treatment (group C) resulted in statistically significant lowering of Significant changes in Aβ 1-40 and Aβ 1-42 levels in the FA fraction of the cortex compared to saline-treated transgenic mice (group A) (FIGS. 30A-30B).

### Example 15

### Effect of HCQ and DIM on Aβ Clearance, Tau Phosphorylation, and LPS-Induced Neuroinflammation in Cells

### Methods

Compound preparation: 1000× stock solutions were prepared in DMSO and further diluted in culture medium so that a max final concentration of 0.1% DMSO was present in the wells. Items that did not need DMSO to solubilize were also adjusted to 0.1% DMSO to have the same conditions in all wells. DMSO stocks were aliquoted and stored at −20° C. Working dilutions were always prepared freshly on the day of experiment.

Culture and treatment of BV-2 cells: The murine microglial cell line BV-2 was cultivated in DMEM medium supplemented with 10% FCS, 1% penicillin/streptomycin and 2 mM L-glutamine (culture medium).

For Aβ clearance assay, 20,000 BV-2 cells per well (uncoated 96 well plates) were plated out. After 24 hours, medium was changed to treatment medium (DMEM, 5% FCS, 2 mM L-glutamine) and cells were maintained in treatment medium for the remaining culture period. After changing cells to treatment medium, the test items were administered 1 hour before Aβ stimulation (Bachem 4061966; final concentration in well: 200 ng/mL (dilutions in medium)). Cells treated with vehicle, cells treated with Aβ alone, as well as wells with Aβ but no cells served as controls. All wells were handled the same way. After 3 h of Aβ stimulation, cell supernatants were collected for the Aβ measurement and cells were carefully washed twice with PBS and thereafter lysed in 35 μl cell lysis buffer (50 mM Tris-HCl, pH 7.4, 150 mM NaCl, 5 mM EDTA, 1% SDS) supplemented with protease inhibitors. Cells were incubated for 15 min at RT on a plate shaker, samples were frozen at −80° C. until further use. The experiment was performed in n=6 technical replicates for all groups.

For LPS stimulation assay, 5000 BV-2 cells per well (uncoated 96 well plates) were plated out. After 48 hours, medium was changed to treatment medium (DMEM, 5% FCS, 2 mM L-glutamine) and cells were maintained in treatment medium for the remaining culture period. After changing cells to treatment medium, the HCQ/DIM concentrations were administered 1 hour before LPS stimulation (Sigma-Aldrich; L6529; 1 mg/ml stock in ddH₂O, final concentration in well: 100 ng/ml (dilutions in medium)). Cells treated with vehicle, cells treated with LPS alone, as well as cells treated with LPS plus reference item (dexamethasone, 10 μM) served as controls. All wells were handled the same way. After 24 h of stimulation, cell supernatants were collected for the cytokine measurement and cells were subjected to MTT assay. The experiment was performed in n=6 technical replicates for all groups.

Culture and treatment of H4-hAPP cells: H4-hAPP cells were thawed and cultivated in Opti-MEM supplemented with 10% FCS, 1% penicillin/streptomycin 200 μg/ml Hygromycin B and 2.5 μg/ml Blasticidin S (=culture medium). H4-hAPP cells were seeded into 96 well plates (2×104 cells per well). On the next day, cells in 96 well plates were treated with T.I., R.I. (DAPT 400 nM) or vehicle. 24 h later, supernatants were collected for further Aβ measurements by MSD. The experiment was performed in n=6 technical replicates for all groups.

Culture and treatment of SH-SY5Y-hTau441(V337M/R406W) cells: SH-SY5Y-hTau441(V337M/R406W) cells were kept in culture medium (DMEM medium, 10% FCS, 1% NEAA, 1% L-Glutamine, 100 μg/ml Gentamycin, 300 μg/ml Geneticin G-418) for ˜2 days until 80-90% confluency. Next, cells were differentiated in culture medium supplemented with 10 μM retinoic acid (RA) for 5 days changing medium every 2 to 3 days. Prior to the treatment, cells were seeded onto 48-well plates at a cell density of 1×105 cells per well (DIV1). Next day (DIV2), HCQ/DIM at 3 concentrations, and RI2 (CHIR99021) were applied. Additionally, the appropriate vehicle control of the test items (0.1% DMSO) was investigated. After 24 h of incubation (DIV3), cells on 48-well plates were harvested. For this purpose, cells were washed once with cold PBS and harvested in 60 μL RIPA-Buffer [50 mM Tris pH 7.4, 1% Nonidet P40, 0.25% Na-deoxy-cholate, 150 mM NaCl, 1 mM EDTA supplemented with freshly added 1 μM NaF, 0.2 mM Na-ortho-vanadate, 80 μM Glycerophosphate, protease (Calbiochem) and phosphatase (Sigma) inhibitor cocktail]. Protein concentration was determined by BCA assay and samples were adjusted to a uniform total protein concentration. Total Tau and phosphorylated Tau were determined by Mesoscale Discovery. The experiments were performed in six technical replicates for all groups.

Aβ42 measurement in BV-2 cells: Supernatants were diluted 1:250, cell lysates 1:2.5 and analyzed for human Aβ42 with MSD® V-PLEX Human Aβ42 Peptide (6E10) Kit (K151LBE, Mesoscale Discovery). The immune assay was carried out according to the manual and plates were read on the MESO QuickPlex SQ 120. Analyte levels were evaluated according to adequate Aβ peptide standards (MSD) as pg per mL. Protein concentration in the cell lysates was determined by BCA assay for potential normalization of Aβ42 levels.

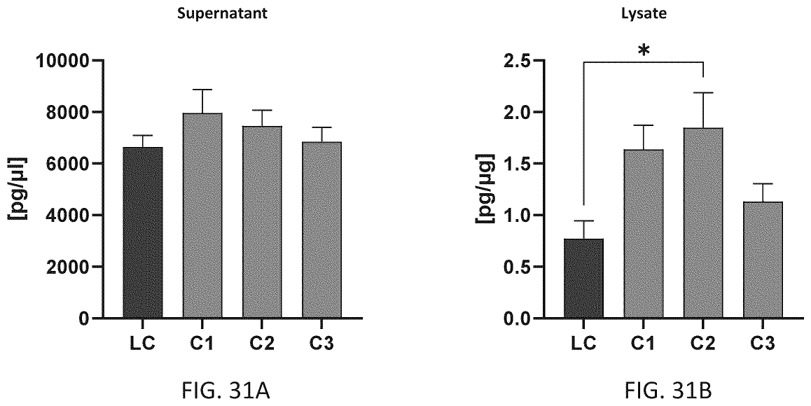

Results: HCQ sulfate/DIM—c1=25 μM HCQ/10 μM DIM, c2=25 μM HCQ/3 μM DIM, c3=10 μM HCQ/10 μM DIM. Treatment at none of the concentrations led to a significant reduction of Aβ1-42 in the supernatant (FIG. 31A). However, significant increase of intracellular Aβ1-42 could be detected in the lysate of cells treated with the second highest concentration (FIG. 31B).

Aβ38, 40, 42 measurement in H4-hAPP cells: Supernatants were diluted 1:10 and analyzed for human Aβ38, 40, and 42 with MSD® 96-well MULTI-SPOT® 6E10 Abeta Triplex Assay (Mesoscale Discovery). The immune assay was carried out according to the manual and plates were read on the MESO QuickPlex SQ 120. Analyte levels were evaluated according to adequate Aβ peptide standards (MSD) as pg per mL.

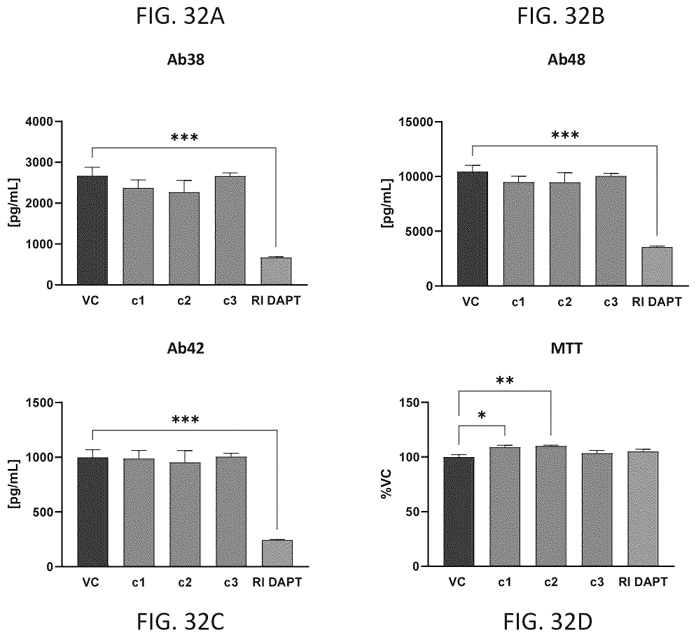

Results: Neuroblastoma cells H4 overexpressing hAPP were treated with HCQ sulfate and DIM at 3 concentrations: c1=25 μM HCQ/10 μM DIM, c2=25 μM HCQ/3 μM DIM, c3=10 μM HCQ/10 μM DIM. AB secretion into the supernatant was assessed 24 h after treatment start. Aβ1-38, 1-40 and 1-42 were assessed using MSD assay. All values were well within the detection range of the assay, whereas Aβ1-40 was the most prominent Aβ species, as expected. The reference item DAPT (gamma-secretase inhibitor) significantly reduced all tested species similarly on all plates. None of the concentrations of HCQ/DIM had any significant effect on Aβ levels, the reference Item significantly reduced all Aβ species. No toxicity of the test item could be seen in the MTT assay (FIGS. 32A-32D).

Total Tau and phosphorylated Tau assay: Total Tau and phosphorylated Tau at residue 231 in RIPA extracts from the SH-SY5Y-hTau441(V337M/R406W9 cells were determined by immunosorbent assay (Phospho(Thr231)/Total Tau Kit K15121D, Mesoscale Discovery). The assay was carried out according to the instructions of the manufacturer. Data was evaluated in comparison to calibration curves provided in the kit and expressed as pg per μg total protein for total Tau and arbitrary units for pTau231.

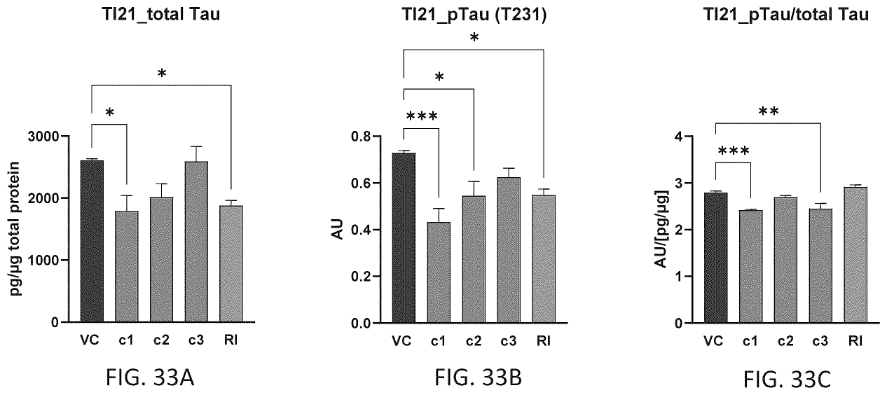

Results: SH-SY5Y-hTau441cells were treated with retinoic acid and with HCQ sulfate and DIM at 3 concentrations: c1=25 μM HCQ/10 μM DIM, c2=25 μM HCQ/3 μM DIM, c3=10 μM HCQ/10 μM DIM. Intracellular levels of Tau and phosphorylated Tau were assessed. Reference item CHIR (GSK3 inhibitor) significantly reduced level of phosphorylated Tau compared to vehicle control. Data were normalized to total protein content accounting for differences in cell number or viability. Treatment at the first concentrations led to a significant decrease of total Tau. The first two concentrations led to a significant decrease of pTau (T231). The pTau (T231) to total tau ratio was decreased at the first and third concentrations (FIGS. 33A-33C). As results were normalized to total protein content, results are not impacted by possible toxicity.

Cytokine measurement: Levels of 10 cytokines (IFN-γ, IL-1β, IL-2, IL-4, IL-5, IL-6, KC/GRO, IL-10, IL-12p70, and TNF-α) were measured in supernatants (1:2 diluted) of BV-2 cells collected 24 h after LPS stimulation. Cytokines were measured by an immunosorbent assay (V-PLEX Proinflammatory Panel 1 Mouse Kit, K15048D, Mesoscale) according to the instructions of the manufacturer and evaluated in comparison to calibration curves provided in the kit. Results are given as pg per mL.

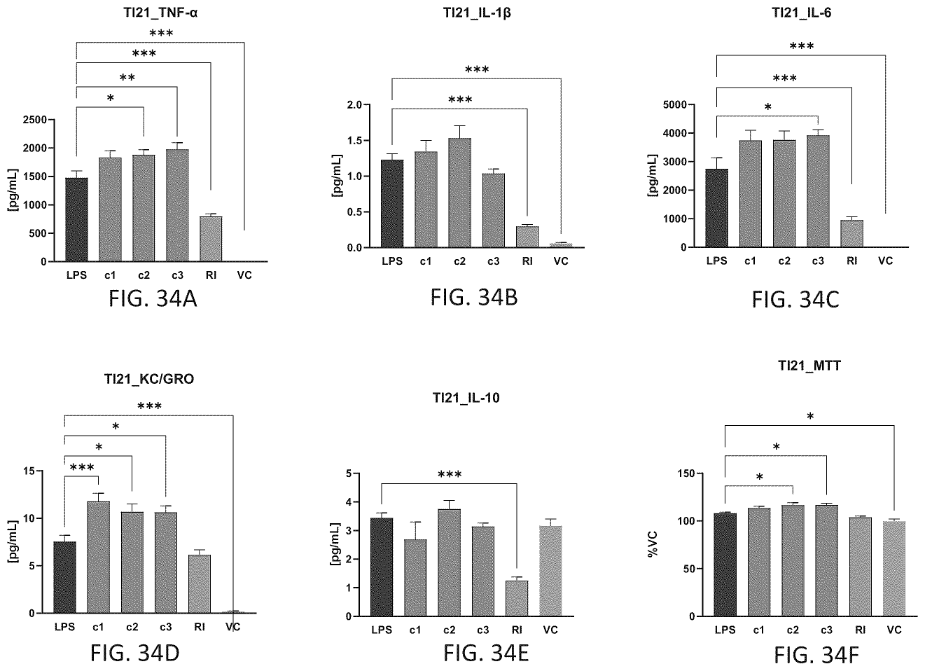

Results: Microglial BV2 cells were stimulated with LPS and cytokine secretion as well as cell viability were assessed (FIGS. 34A-34F). As expected, a significant increase of pro-inflammatory cytokines due to LPS stimulation was observed, which could be reversed with RI dexamethasone treatment on all plates. Treatment with HCQ sulfate/DIM was performed at 3 concentrations: c1=25 μM HCQ/10 μM DIM, c2=25 μM HCQ/3 μM DIM, c3=10 μM HCQ/10 μM DIM. Treatment led to significant increase in TNF-α at all concentrations (FIG. 34A). IL-6 levels were significantly increased at the lowest concentration FIG. 34C). KC/GRO was significantly increased at all concentrations (FIG. 34D). The MTT Assay indicated higher viability of samples treated with low and middle dose of HCQ/DIM, which may be associated with the increased cytokine secretion (FIG. 34F).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising:
administering to a subject an amount of an active agent effective to at least partially normalize an aberrant level of one or more indicators in the brain, cerebrospinal fluid, and/or blood, wherein the indicators comprise extracellular amyloid beta (Aβ) concentration, tau phosphorylation, neuroinflammation, plasma neurofilament-light (NfL) concentration, hippocampal synaptic plasticity, or any combination thereof.

2. The method of claim 1, wherein normalizing the aberrant level of the one or more indicators reduces extracellular Aβ concentration, decreases tau phosphorylation, reduces neuroinflammation, decreases plasma NfL concentration, increases hippocampal synaptic plasticity, or any combination thereof.

3. The method of claim 2, wherein reducing extracellular Aβ concentration comprises reducing Aβ secretion, increasing Aβ clearance, or both.

4. The method of claim 2, wherein reducing neuroinflammation comprises reducing a concentration of interleukin-6, interleukin-1β, interleukin-12p70, interleukin-10, tumor necrosis factor alpha, or any combination thereof.

5. The method of claim 1, wherein the active agent comprises hydroxychloroquine, tauroursodeoxycholic acid (TUDCA), C188-9 (TTI-101, N-[4-hydroxy-3-(2-hydroxynaphthalen-1-yl)naphthalen-1-yl]-4-methoxybenzenesulfonamide), dasatinib, MLS-0437605 (N-4-fluoro-1,3-benzothiazol-2-71)-5-(4-methoxyphenyl)-1,3,4-oxadiazol-2-amine), a methyl-β-d-galactomalonyl phenyl ester, irinotecan, pyrimethamine, TTI-102,3,3′-diindolylmethane (DIM), or any combination thereof.

6. The method of claim 5, wherein the active agent comprises hydroxychloroquine, TUDCA, DIM, or any combination thereof.

7. The method of claim 5, wherein the active agent comprises hydroxychloroquine nanoparticles.

8. The method of claim 1, wherein the active agent at least partially normalizes an aberrant level of extracellular Aβ concentration, plasma NfL concentration, hippocampal synaptic plasticity, or any combination thereof.

9. The method of claim 8, wherein the active agent comprises hydroxychloroquine, TUDCA, DIM, or any combination thereof.

10. The method of claim 9, wherein the active agent comprises a combination of hydroxychloroquine and TUDCA, and at least partially normalizing the aberrant level of extracellular Aβ concentration comprises reducing Aβ secretion and increasing Aβ clearance.

11. The method of claim 1, wherein the active agent at least partially normalizes the level of at least two of the one or more indicators.

12. The method of claim 11, wherein the active agent reduces neuroinflammation and extracellular Aβ concentration.

13. The method of claim 1, wherein the active agent inhibits tau phosphorylation.

14. The method of claim 13, wherein the active agent comprises dasatinib, C188-9, hydroxychloroquine, or a combination thereof.

15. The method of claim 1, further comprising receiving data comprising an initial level of at least one of the indicators prior to administering the active agent to the subject.

16. The method of claim 1, further comprising:
receiving data comprising a post-administration level of at least one of the indicators following administration of the active agent to the subject; and
selecting an adjusted amount of the active agent for administration to the subject based at least in part on the post-administration level.

17. The method of claim 1, wherein the subject is diagnosed as having Alzheimer's disease (AD) prior to administering the active agent.

18. The method of claim 1, further comprising administering the active agent to the subject prophylactically in the absence of any cognitive, behavioral, mood, or psychological signs or symptoms of AD.

19. A method for inhibiting progression of AD, comprising
administering to a subject diagnosed as having AD an amount of an active agent effective to at least partially normalize an aberrant level of one or more indicators of Alzheimer's disease pathology wherein the indicators comprise extracellular amyloid beta (Aβ) concentration, tau phosphorylation, neuroinflammation, plasma NfL concentration, hippocampal synaptic plasticity, or any combination thereof, and wherein the active agent comprises hydroxychloroquine, TUDCA, DIM, C188-9, dasatinib, MLS-0437605, a methyl-β-d-galactomalonyl phenyl ester, irinotecan, pyrimethamine, TTI-102, or any combination thereof.

20. A method for inhibiting or preventing development of AD, comprising:
identifying a subject as being at risk of developing AD by (i) identifying the subject as being an APOE ε4 carrier, or (ii) identifying the subject as having an elevated level of one or more indicators in the brain relative to a normal level of the one or more indicators in the brain, or (iii) identifying the subject as having an aberrant level of one or more indicators in cerebrospinal fluid and/or blood assays relative to a normal level of the one or more indicators in the cerebrospinal fluid and/or blood, or (iv) any combination of (i), (ii), and (iii), wherein the indicators comprise extracellular amyloid beta (Aβ) concentration, tau phosphorylation, neuroinflammation, plasma NfL concentration, hippocampal synaptic plasticity, or any combination thereof; and
administering to the subject at risk of developing AD an amount of an active agent effective to at least partially normalize an aberrant level of the one or more indicators of Alzheimer's disease pathology, wherein the active agent comprises hydroxychloroquine, TUDCA, DIM, C188-9, dasatinib, MLS-0437605, a methyl-β-d-galactomalonyl phenyl ester, irinotecan, pyrimethamine, TTI-102, or any combination thereof.
